(19) United States
(12) Patent Application Publication
(10) Pub. No.: US 2025/0316355 A1
(43) Pub. Date: Oct. 9, 2025

(54) HEALTHCARE SYSTEM FOR AND METHODS OF MANAGING BRAIN INJURY OR CONCUSSION

(71) Applicant: SPORTGAIT, INC., Wilmington, NC (US)

(72) Inventors: Kevin CARNEIRO, Chapel Hill, NC (US); Anthony VOLPE, Chapel Hill, NC (US); Steve DEVRIEZE, Chapel Hill, NC (US)

(21) Appl. No.: 18/871,315

(22) PCT Filed: Jun. 2, 2023
(86) PCT No.: PCT/US2023/024374

§ 371 Date: Dec. 3, 2024

Related U.S. Application Data
(60) Provisional application No. 63/349,117, filed on Jun. 5, 2022.
(60) Provisional application No. 63/405,198, filed on Sep. 9, 2022.

(51) Int. Cl. G16H 20/00; G06N 3/08; G16H 10/20; G16H 10/60; G16H 50/20; G16H 50/70

(52) U.S. Cl. CPC G16H 20/00; G06N 3/08; G16H 10/20; G16H 10/60; G16H 50/20; G16H 50/70

(57) ABSTRACT

In some aspects, the present disclosure provides a computer-implemented method for predicting a plurality of treatment options for treatment of a traumatic brain injury for a subject, the computer-implemented method comprising: (a) receiving a plurality of attributes of the subject, wherein the plurality of attributes is related to the traumatic brain injury; and (b) applying a machine learning model to the plurality of attributes to predict (i) a clinical outcome comprising a traumatic brain injury, and (ii) the plurality of treatment options for treatment of the traumatic brain injury.

### CROSS-REFERENCE

This application claims the benefit of U.S. Provisional Application No. 63/349,117, filed Jun. 5, 2022 and U.S. Provisional Application No. 63/405,198, filed Sep. 9, 2022, each of which are incorporated herein by reference.

### FIELD OF THE INVENTION

The presently disclosed subject matter relates generally to healthcare systems and more particularly to a healthcare system for and methods of managing brain injury or concussion.

### BACKGROUND

Concussions can be classified as a mild traumatic brain injury. Concussions in athletics is an ubiquitous health concern, which can occur in a wide range of sports and affect all kinds of athletes, both professional players and young athletes. With respect to treating a mild traumatic brain injury (mTBI) or concussion, healthcare providers are frequently focused on the diagnosis and a “hands off” approach to treatment, which is often a treatment regimen of rest only.

### SUMMARY

In some embodiments, the healthcare system and methods may provide a digital health platform that may capture and leverage clinically customized structured data to enable machine learning (ML) treatment insights from real-world concussion patient data.

In some embodiments, the healthcare system and methods may provide a digital health platform including machine learning models that may be trained to generate phenotypes based on patient and injury characteristics and symptoms across certain clinical domains, such as, but not limited to, behavioral, cervicogenic, cognitive, headache, ocular, physiologic, sleep-wake, and vestibular.

In some embodiments, the healthcare system for and methods may provide a brain healthcare application running on an application server and accessible in a networked computing environment.

In some embodiments, the healthcare system for and methods may provide a brain healthcare application including a machine learning component.

In some embodiments, the healthcare system for and methods may provide a brain healthcare application including multiple algorithms, such as, but not limited to, a machine learning algorithm, a persistent concussion symptoms (PCS) prediction algorithm, and a clustering algorithm.

In some embodiments, the healthcare system for and methods may provide a brain healthcare application including robust analytics utilizing world class machine learning to inform an optimal treatment plan for similar patient segments and similar concussion or brain injury types.

In some embodiments, the healthcare system for and methods may provide a brain healthcare application including a clinician web portal that may be a custom interface used by clinicians or healthcare providers.

In some embodiments, the healthcare system for and methods may provide a clinician web portal including a structured data capture intake form to be used at diagnosis and during the patient treatment process.

In some embodiments, the healthcare system for and methods may provide a brain healthcare application including a patient mobile app (e.g., brain health mobile app) that may be a custom interface used by patients.

In some embodiments, the healthcare system for and methods may provide a clinician web portal featuring clinically customized concussion data capture for the purposes of enabling:
(1) machine learning algorithms to phenotype concussions based on clustering numerous data attributes (e.g., patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, physical exam results, wearable physiologic data, imaging data, history of previous recovery trajectories and treatments, heart rate variability, physical activity data, blood pressure, perspiration, calories burned, steps walked, body temperature, injury characteristics, symptoms, previous treatments, previous recovery timelines, or any combination thereof);
(2) machine learning algorithms to predict recovery timelines and generate treatment insights for the clinician (i.e., based on the phenotype) that can support the care plan; and
(3) integrated technology solution which links data between the clinician web portal, electronic medical records (EMR), and a patient mobile app, which enables patient reported recovery data.

In some embodiments, the healthcare system for and methods may provide a communication platform for the patient, caregiver, and/or healthcare multidisciplinary team, including the customized clinician web portal and/or the customized patient mobile app.

In some embodiments, the healthcare system for and methods may provide a brain healthcare application that features (1) structured data intake, (2) data aggregation, (3) machine learning model, and (4) dashboard reports and insights.

In some embodiments, the healthcare system for and methods may provide a brain healthcare application for managing mild traumatic brain injury (mTBI) or concussion.

In some embodiments, the healthcare system for and methods may provide a brain healthcare platform for delivering personalized treatment, including real-time feedback and data collection.

In some embodiments, the healthcare system for and methods may provide a brain healthcare platform for providing customized treatment insights for improved management of concussion symptoms compared with the standard of care (SOC) alone.

In some embodiments, the healthcare system for and methods may provide a flexible platform for managing multiple other healthcare needs, such as, but not limited to, sports injuries, COVID risk assessment, back pain, musculoskeletal conditions and stroke, among others.

In some aspects the present invention is directed to a computer-implemented method for predicting a plurality of treatment options for treatment of a traumatic brain injury for a subject, the computer-implemented method comprising: (a) receiving a plurality of attributes of the subject, wherein the plurality of attributes is related to the traumatic brain injury; and (b) applying a machine learning model to the plurality of attributes to predict (i) a clinical outcome comprising a traumatic brain injury, and (ii) the plurality of treatment options for treatment of the traumatic brain injury.

In some embodiments, the receiving of the plurality of attributes is via a graphical user interface (GUI) of an electronic device.

In some embodiments, the subject or a medical professional enters the plurality of attributes using the GUI

In some embodiments, the receiving the plurality of attributes is performed autonomously.

In some embodiments, the receiving the plurality of attributes is via a wearable device.

In some embodiments, the wearable device comprises a bracelet, a computer, a mobile device, an epidermal, an earbud, a headphone, a fabric, a shoe, an implant, glasses, a tracker, a belt, a sock, a shirt, or a ring.

In some embodiments, the plurality of attributes comprises past medical events of the subject.

In some embodiments, the plurality of attributes indicates a presence or absence of a symptom in the subject.

In some embodiments, the plurality of attributes indicates a severity or mildness of a symptom in the subject.

In some embodiments, the plurality of attributes comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, physical exam results, wearable physiologic data, imaging data, history of previous recovery trajectories and treatments, heart rate variability, physical activity data, blood pressure, perspiration, calories burned, steps walked, body temperature, or any combination thereof of the subject.

In some embodiments, the method further comprising classifying the traumatic brain injury as a concussion.

In some embodiments, the method further comprising classifying the concussion as a concussion phenotype.

In some embodiments, the concussion phenotype comprises a persistent concussion.

In some embodiments, the method further comprising generating a probability that the traumatic brain injury is a concussion.

In some embodiments, the method further comprising generating a plurality of probabilities for a plurality of concussion phenotypes of the traumatic brain injury.

In some embodiments, the method further comprising predicting, by the machine learning model, a recovery timeline for the subject, based at least in part on the plurality on attributes.

In some embodiments, the method further comprising selecting a treatment in the plurality of treatment options.

In some embodiments, the treatment is personalized to the subject.

In some embodiments, the method further comprising delivering or administering the treatment to the subject.

In some embodiments, the plurality of attributes comprises activity data of the subject, wherein the activity data relates to whether the subject has adhered to a current treatment.

In some embodiments, the method further comprising selecting a treatment in the plurality of treatment options for the subject based on at least in part on the activity data of the subject.

In some embodiments, the treatment is different from a previous treatment delivered to the subject for the traumatic brain injury.

In some embodiments, the treatment is delivered or administered to the subject with a duration or a frequency that is different than a previous duration or a previous frequency of a previous treatment delivered to the subject for the traumatic brain injury.

In some embodiments, the treatment is performed by the subject with the duration or the frequency that is different than the previous duration or the previous frequency of a previous treatment delivered to the subject for the traumatic brain injury.

In some embodiments, the method further comprising training the machine learning model by: (a) receiving a dataset comprising (i) a plurality of attributes of a plurality of reference subjects, and (ii) a plurality of clinical outcomes for the plurality of subjects; and (b) processing a reference dataset using the machine learning model to generate a plurality of outputs and (ii) updating a parameter of the machine learning model based on a loss function, wherein the loss function is based on the plurality of clinical outcomes and the plurality of outputs, wherein the plurality of outputs is indicative of a plurality of clinical outcome predictions.

In some embodiments, the plurality of outputs parameterizes the plurality of clinical outcome predictions.

In some aspects the present invention is directed to a computer-implemented method for training a machine learning model, comprising: (a) receiving a dataset comprising (i) a plurality of attributes of a plurality of reference subjects and (ii) a plurality of clinical outcomes for the plurality of reference subjects that received a plurality of traumatic brain injury treatments; and (b) training the machine learning model by (i) processing the dataset using the machine learning model to generate a plurality of outputs and (ii) updating a parameter of the machine learning model based on a loss function, wherein the loss function is based on the plurality of clinical outcomes and the plurality of outputs, wherein the plurality of outputs is indicative of an effectiveness of the plurality of traumatic brain injury treatments for the plurality of reference subjects.

In some embodiments, the plurality of attributes comprises past medical events of the plurality of reference subjects.

In some embodiments, the plurality of attributes indicates a presence or absence of a symptom in the plurality of reference subjects.

In some embodiments, the plurality of attributes indicates a severity or mildness of a symptom in the plurality of reference subjects.

In some embodiments, the plurality of attributes comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, demographic characteristics, injury characteristics, physical exam results, wearable physiologic data, imaging data, history of previous recovery trajectories and treatments, or any combination thereof of the subject.

In some aspects the present invention is directed to a computer-implemented method for training a machine learning model, comprising: (a) receiving a dataset comprising (i) a plurality of attributes of a plurality of reference subjects and (ii) a plurality of clinical outcomes for the plurality of reference subjects that were afflicted with a traumatic brain injury; and (b) training the machine learning model by (i) processing the dataset using the machine learning model to generate a plurality of outputs and (ii) updating a parameter of the machine learning model based on a loss function, wherein the loss function is based on the plurality of clinical outcomes and the plurality of outputs, wherein the plurality of outputs is indicative of a recovery phenotype of the traumatic brain injury for the plurality of reference subjects.

In some embodiments, the plurality of outputs comprises a plurality of latent representations for the plurality of attributes for the plurality of reference subjects.

In some embodiments, the method further comprising clustering the plurality of latent representations identify the recovery phenotype for the plurality of reference subjects.

In some embodiments, the method further comprising applying the machine learning model to a subject not among the plurality of reference subjects to classify the recovery phenotype of the subject.

In some embodiments, the plurality of attributes indicates a presence or absence of a symptom in the plurality of reference subjects.

In some embodiments, the plurality of attributes indicates a severity or mildness of a symptom in the plurality of reference subjects.

In some embodiments, the plurality of attributes comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, demographic characteristics, injury characteristics, physical exam results, wearable physiologic data, imaging data, history of previous recovery trajectories and treatments, or any combination thereof of the subject

In some embodiments, the recovery phenotype comprises a concussion recovery phenotype.

In some embodiments, the concussion recovery phenotype comprises a persistent concussion.

In some embodiments, the method further comprising generating a probability that the recovery phenotype is a concussion recovery phenotype.

In some aspects the present invention is directed to a computer-implemented method for optimizing a treatment for a traumatic brain injury in a subject, the method comprising: (a) receiving activity data of the subject, wherein the subject has received a treatment for the traumatic brain injury, and wherein the activity data relates to whether the subject has adhered to the treatment; and (b) applying a machine learning model to the activity data to predict whether the subject should continue the treatment or switch to a different treatment.

In some embodiments, the receiving of the activity data is via a graphical user interface (GUI) of an electronic device.

In some embodiments, the subject or a medical professional enters the plurality of attributes using the GUI.

In some embodiments, the receiving the plurality of attributes is performed autonomously.

In some embodiments, the receiving the plurality of attributes is via a wearable device.

In some embodiments, the wearable device comprises a bracelet, a computer, a mobile device, an epidermal, an earbud, a headphone, a fabric, a shoe, an implant, glasses, a tracker, a belt, a sock, a shirt, or a ring.

In some embodiments, the activity data relates to whether the subject has adhered to a current treatment.

In some embodiments, the activity data indicates a presence or absence of a symptom in the subject.

In some embodiments, the activity data indicates a severity or mildness of a symptom in the subject.

In some embodiments, the activity data comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, history of previous recovery trajectories and treatments, or any combination thereof of the subject.

In some embodiments, the method further comprising delivering or administering the different treatment to the subject.

In some embodiments, the different treatment is different from the treatment in a duration or a frequency.

In some embodiments, the method further comprising training the machine learning model by: (a) receiving a dataset comprising (i) a plurality of attributes of a plurality of subjects, (ii) a plurality of activity data that relates to whether the subject has adhered to the treatment, and (iii) a plurality of clinical outcomes for the plurality of subjects; and (b) training the machine learning model by (i) processing the dataset using the machine learning model to generate a plurality of outputs and (ii) updating a parameter of the machine learning model based on a loss function, wherein the loss function is based on the plurality of clinical outcomes and the plurality of outputs, wherein the plurality of outputs comprises or parameterizes a plurality of clinical outcome predictions.

In some aspects the present invention is directed to a computer-implemented method for generating an expected clinical outcome of a subject having a concussion, comprising: (a) receiving a plurality of attributes of the subject, wherein the plurality of attributes is related to a traumatic brain injury; and (b) applying a machine learning model to the plurality of attributes to predict a recovery timeline and an uncertainty value associated with the recovery timeline.

In some embodiments, the recovery timeline comprises a timeline of one or more symptoms.

In some embodiments, the recovery timeline comprises a timeline for one or more concussion phenotypes.

In some embodiments, the recovery timeline comprises a plurality of uncertainty values.

In some embodiments, the receiving of the plurality of attributes is via a graphical user interface (GUI) of an electronic device.

In some embodiments, the subject or a medical professional enters the plurality of attributes using the GUI.

In some embodiments, the receiving the plurality of attributes is performed autonomously.

In some embodiments, the receiving the plurality of attributes is via a wearable device.

In some embodiments, the wearable device comprises a bracelet, a computer, a mobile device, an epidermal, an earbud, a headphone, a fabric, a shoe, an implant, glasses, a tracker, a belt, a sock, a shirt, or a ring.

In some embodiments, the plurality of attributes comprises past medical events of the subject.

In some embodiments, the plurality of attributes indicates a presence or absence of a symptom in the subject.

In some embodiments, the plurality of attributes indicates a severity or mildness of a symptom in the subject.

In some embodiments, the plurality of attributes comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, history of previous recovery trajectories and treatments, or any combination thereof of the subject or any combination thereof of the subject.

In some embodiments, the method further comprising training the machine learning model by: (a) receiving a dataset comprising a plurality of attributes and a plurality of recovery timelines for a plurality of reference subjects, wherein the plurality of attributes and the plurality of time-varying clinical outcomes are related to a traumatic brain injury; and (b) training the machine learning model by processing the plurality of attributes using the machine learning model to generate a plurality of outputs and updating a parameter of the machine learning model based on a loss function, wherein the loss function is based on the plurality of recovery timelines and the plurality of outputs, wherein the plurality of outputs indicates a recovery timeline and an uncertainty value associated with the recovery timeline.

In some aspects the present invention is directed to a platform comprising: (a) a client device comprising at least one processor, an operating system configured to perform executable instructions, a memory, and a computer program including instructions executable by the client device to create an application comprising a software module for receiving a plurality of attributes of a subject, wherein the plurality of attributes is related to a traumatic brain injury of the subject; and (b) a server comprising at least one processor, an operating system configured to perform executable instructions, a memory, and a computer program including instructions executable by the client device to create an application comprising a software module for applying a machine learning model to the plurality of attributes to predict (i) a clinical outcome comprising a traumatic brain injury, and (ii) a plurality of treatment options for treatment of the traumatic brain injury.

In some embodiments, the client device comprises a mobile electronic device.

In some embodiments, the plurality of attributes comprises one or more recovery statistics of the subject.

In some embodiments, the one or more recovery statistics of the subject are configured to be received from the subject.

In some embodiments, the application further comprises a video player configured to provide one or more instructional videos for performing one or more exercises for treating the traumatic brain injury of the subject.

In some aspects the present invention is directed to a computer-implemented system comprising a computing device comprising at least one processor, an operating system configured to perform executable instructions, a memory, and a computer program including instructions executable by the computing device to create an application comprising: (a) a software module configured to receive a plurality of attributes of the subject, wherein the plurality of attributes is related to a traumatic brain injury; and (b) a software module configured to apply a machine learning model to the plurality of attributes to predict (i) a clinical outcome comprising a traumatic brain injury, (ii) a recovery phenotype of the traumatic brain injury, or (iii) a plurality of treatment options for treatment of the traumatic brain injury.

In some aspects the present invention is directed to a non-transitory computer-readable storage media encoded with a computer program including instructions executable by one or more processors to generate a selection of treatment options for a subject comprising: (a) a database, in a computer memory, comprising a plurality of attributes of the subject, wherein the plurality of attributes is related to a traumatic brain injury; and (b) a software module configured to apply a machine learning model to the plurality of attributes to predict (i) a clinical outcome comprising a traumatic brain injury, (ii) a recovery phenotype of the traumatic brain injury, or (iii) a plurality of treatment options for treatment of the traumatic brain injury.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a block diagram of an example of the healthcare system for managing brain injury or concussion;

FIG. 2 illustrates a flow diagram of an example of an overall process flow of the healthcare system for managing brain injury or concussion;

FIG. 3 illustrates a latent space for traumatic brain injury phenotypes;

FIG. 4 illustrates a block diagram of an example of a system data flow and architecture of the healthcare system for managing brain injury or concussion;

FIG. 5 illustrates a flow diagram of an example of a clinician web portal flow of the healthcare system for managing brain injury or concussion;

FIG. 6 illustrates a flow diagram of an example of a clinician workflow of the healthcare system for managing brain injury or concussion;

FIG. 7 illustrates a flow diagram of an example of a patient portal (or mobile app) of the healthcare system for managing brain injury or concussion;

FIG. 8 through FIG. 54 show screenshots of an example of a process of using the clinician web portal and/or the brain health mobile app of the healthcare system for managing brain injury or concussion; and

FIG. 55A through FIG. 61 show screenshots of an example of the information structure supporting the clinician web portal and/or patient portal (or mobile app) of the healthcare system for managing brain injury or concussion.

### DETAILED DESCRIPTION

With respect to treating a mild traumatic brain injury (mTBI) or concussion, healthcare providers are frequently focused on the diagnosis and a “hands off” approach to treatment, which is often a treatment regimen of rest only. That is, a healthcare provider may determine whether a person has a concussion or not, but often do not provide customized treatment plans for the concussed or otherwise affected by mTBI. In some cases the healthcare provider may refer concussed or mTBI patients to other specialists, given a concussion or mTBI can involve many clinical domains, such as headache, neck pain, vision issues, balance issues, sleeping issues, neural cognitive issues, behavioral symptoms, physiologic symptoms, and the like. However, healthcare providers are often limited to assessing and/or treating one or a few clinical domains only. Therefore, new approaches are needed with respect to treating mTBI or concussion. In some aspects, the presently disclosed subject matter provides a healthcare system for and methods of managing brain injury or concussion which can be used overcome some of the challenges in diagnosing and treating patients affected by mTBI or concussion.

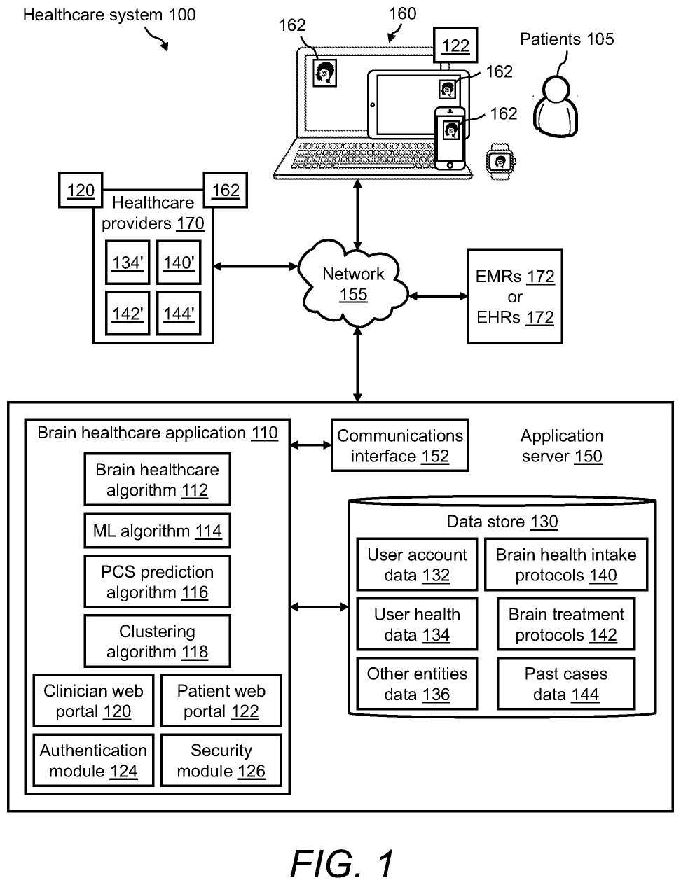

Referring now to FIG. 1 is a block diagram of an example the healthcare system 100 for managing brain injury or concussion. In this example, healthcare system 100 may be provided in a networked computing configuration that includes a brain healthcare application 110 and a data store 130 running on an application server 150.

At application server 150, brain healthcare application 110 may further include a brain healthcare algorithm 112, a ML algorithm 114, a persistent concussion symptoms (PCS) prediction algorithm 116, a clustering algorithm 118, a clinician web portal 120, a patient web portal 122, an authentication module 124, and a security module 126. Further, brain healthcare application 110 may include a machine learning component, such as ML algorithm 114.

Further, user account data 132, user health data 134, other entities data 136, brain health intake protocols 140, brain treatment protocols 142, and past cases data 144 may be stored at data store 130. Additionally, application server 150 may be accessible via a network 155. Network 155 may be, for example, a local area network (LAN) and/or a wide area network (WAN) for connecting to the Internet or to an Intranet. Application server 150 may connect to network 155 by any wired and/or wireless means. Application server 150 may include, for example, a communications interface 152.

A plurality of patients (or users) 105 may be associated with the healthcare system 100. In healthcare system 100, patients 105 may be any person (or user) seeking help in the area of brain health. For example, patients 105 may be any patients seeking treatment for mild traumatic brain injury (mTBI), or concussion. The patients 105 may access brain healthcare application 110 at application server 150 via their respective user computers 160 and network 155. User computers 160 may be any computing device, such as, but not limited to, a desktop computer, a laptop computer, a handheld computing device, a mobile phone (or smart phone), a tablet device, a smartwatch, and the like. Any information about patients 105 may be stored in user account data 132 and/or user health data 134 at data store 130 of application server 150. For example, a user profile for each patient 105 in user account data 132 may include, for example, account information, user name, user credentials, user payment information, and the like. Further, user health data 134 may include any information about the health of individual patients 105. The health information may include, for example, general health information, concussion-specific health information, family history with respect to concussion, and the like.

Healthcare system 100 may also include one or more healthcare providers 170. Healthcare providers 170 may be, for example, any healthcare providers with expertise in mTBI or concussion. Further, healthcare providers 170 may be those working with patients (or users) 105 seeking treatment for mTBI or concussion. Further, in healthcare system 100, electronic medical records (EMR) 172 or electronic health records (EHR) 172 may be accessible by patients 105 and/or healthcare providers 170 via network 155. Examples of EMR/EHR systems may include, but are not limited to, Epic Systems Corporation (Verona, WI) and CareCloud, Inc (Somerset, NJ).

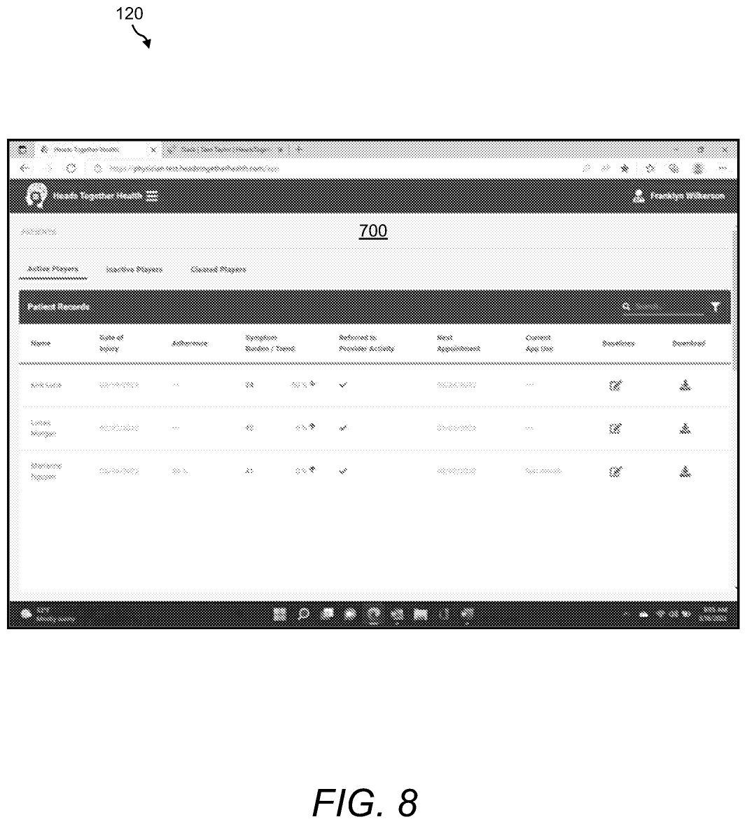

Brain healthcare application 110 at application server 150 may be a software application that may be implemented as a web application and run in a web browser, such as Google Chrome or Microsoft Edge. In one example, healthcare providers 170 may interact with brain healthcare application 110 using clinician web portal 120 at application server 150. Similarly, patients 105 may interact with brain healthcare application 110 using patient web portal 122. For example, clinician web portal 120 and patient web portal 122 may be web-based portals that are accessible via network 155. Example screenshots of clinician web portal 120 are shown and described hereinbelow with reference to FIG. 8 through FIG. 30.

In another example, brain healthcare application 110 at application server 150 may be accessible to patients 105 and healthcare providers 170 via a brain health desktop application 162 or brain health mobile app 162. Brain health desktop application 162 and brain health mobile app 162 is hereafter called brain health mobile app 162.

In one example, brain health mobile app 162 may be implemented, for example, as a NET application, a desktop application, a mobile app, an application program interface (API), and the like. When configured as a mobile app, brain health mobile app 162 may be designed to operate on any device platform, including for example, Windows, Android, Apple, and the like. One operating mode of brain health mobile app 162 may be designed for patients 105 to use.

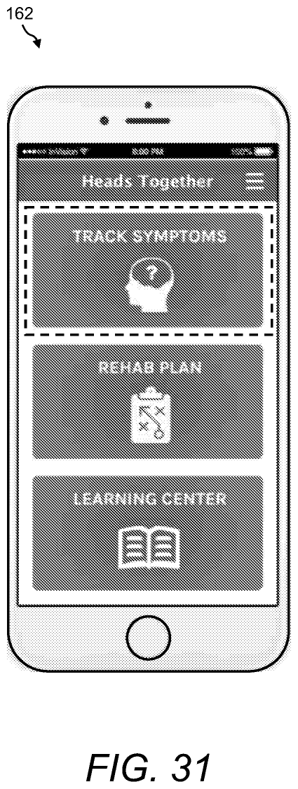

Accordingly, patients 105 may interact with the brain healthcare application 110 using brain health mobile app 162 of their user computer 160 (e.g., smart phone or tablet device). Example screenshots of brain health mobile app 162 are shown and described hereinbelow with reference to FIG. 31 through FIG. 52. Accordingly, patients 105 may interact with brain healthcare application 110 using either patient web portal 122 or brain health mobile app 162.

Further, another operating mode of brain health mobile app 162 may be designed for use by healthcare providers 170. That is, healthcare providers 170 may interact with brain healthcare application 110 using either clinician web portal 120 or brain health mobile app 162. In some embodiments, healthcare system 100 may include one mobile app to be used by patients 105 and then a different mobile app to be used by healthcare providers 170.

Referring to FIG. 1, healthcare providers 170 may access brain healthcare application 110 via the clinician web portal 120, brain health desktop application 162 and/or brain health mobile app 162. For example, using clinician web portal 120, healthcare providers 170 may access to user health data 134, brain health intake protocols 140, brain treatment protocols 142, past cases data 144, and/or EMRs/EHRs 172.

Authentication module 124 of brain healthcare application 110 may be used to manage the authentication process of any entities of healthcare system 100, such as patients 105 and healthcare providers 170. For example, when patients 105 and/or healthcare providers 170 sign into patient web portal 122 and clinician web portal 120, respectively, of brain healthcare application 110, a standard authentication process may be performed that allows access. User-sign in may occur a number of ways. In one example, patients 105 and healthcare providers 170 may use a web browser to access patient web portal 122 and clinician web portal 120, respectively, of brain healthcare application 110 and enter credentials (e.g., username and password).

In another example, patients 105 and healthcare providers 170 may use brain health mobile app 162 to enter his/her credentials. In yet another example, the sign-in process may occur automatically when the patient 105 and/or healthcare provider 170 starts brain health mobile app 162. As patients 105 are authorized to access healthcare system 100, user information may be stored in user account data 132 in data store 130. Similarly, as other entities (e.g., healthcare providers 170) access healthcare system 100, information may be stored in other entities data 136 in data store 130. Additionally, information about any cases of mTBI or concussion that have been treated in the past may be stored in past cases data 144 in data store 130.

Security module 126 of brain healthcare application 110 may be used to perform any system security functions with respect to keeping secure the contents of data store 130 and/or any other information with respect to healthcare system 100. Security module 126 may use standard security techniques, such as encryption, tokenization, secure hashtags (or hash tags), and the like. Data store 130 may be, for example, data repositories (like databases) and/or flat files that can store data. Further, healthcare system 100 is not limited to one data store 130 only. Healthcare system 100 may include multiple data stores 130. Further, data store 130 may be provided on a data server that is separate from application server 150. In healthcare system 100, data store 130 may be termed a machine learning database.

Communications interface 152 at application server 150 may be any wired and/or wireless communication interface for connecting to a network (e.g., network 155) and by which information may be exchanged with other devices connected to the network. Examples of wired communication interfaces may include, but are not limited to, USB ports, RS232 connectors, RJ45 connectors, Ethernet, and any combinations thereof. Examples of wireless communication interfaces may include, but are not limited to, an Intranet connection, Internet, ISM, Bluetooth® technology, Bluetooth® Low Energy (BLE) technology, Wi-Fi, Wi-Max, IEEE 402.11 technology, ZigBee technology, Z-Wave technology, 6LoWPAN technology (i.e., IPv6 over Low Power Wireless Area Network (6LoWPAN)), ANT or ANT+ (Advanced Network Tools) technology, radio frequency (RF), Infrared Data Association (IrDA) compatible protocols, Local Area Networks (LAN), Wide Area Networks (WAN), Shared Wireless Access Protocol (SWAP), any combinations thereof, and other types of wireless networking protocols.

Referring to FIG. 1, healthcare system 100 may operate in a client/server computing architecture, which is well known. In this example, brain healthcare application 110 at the application server 150 may be the server component of healthcare system 100, while brain health mobile app 162 at each of the user computers 160 may be the client component of healthcare system 100. In other words, brain health mobile app 162 at each of the user computers 160 is the counterpart to brain healthcare application 110 at application server 150.

Additionally, application server 150 may be any networked computing configuration as long as it is accessible via network 155 by other entities of healthcare system 100, such as patients 105 and healthcare providers 170. For example, healthcare system 100, and more particularly the brain healthcare application 110 on application server 150, may support a cloud computing environment. In a cloud computing environment, application server 150 may be the cloud server. Further, brain healthcare application 110 is not limited to running on one application server 150 only. Healthcare system 100 may include multiple application servers 150 (or cloud servers) in order to ensure high-availability of computing resources.

Referring to FIG. 1, brain healthcare application 110 may be a software application that provides a means of using brain health intake protocols 140, brain treatment protocols 142, and clinician web portal 120 or brain health mobile app 162 to manage healthcare providers 170 with respect to treating mTBI or concussion.

Additionally, using brain healthcare application 110, ongoing communication between entities of healthcare system 100 may occur throughout brain health intake protocols 140 and/or brain treatment protocols 142. For example, using brain health mobile app 162, push-notifications may be automatically generated to patients 105 and confirmation notifications may be returned from patients 105 to brain healthcare application 110 throughout the entirety of brain health intake protocols 140 and/or brain treatment protocols 142.

Brain healthcare algorithm 112 may be used to process all the information generated and/or received by brain healthcare application 110 and thereby manage the overall operations of healthcare system 100. In one example, brain healthcare algorithm 112, together with ML algorithm 114 and/or PCS prediction algorithm 116, may be used to process user health data 134 with respect to brain health intake protocols 140 in order to determine the best intervention as indicated in brain treatment protocols 142.

Further, ML algorithm 114 of brain healthcare application 110 may be used to optimize patient phenotyping and therapies provided to the patient (or user) 105. For example, the machine learning processes of ML algorithm 114 may use one or more data sets to make certain predictions. For example, data sets in user health data 134, brain health intake protocols 140, brain treatment protocols 142, and past cases data 144 at data store 130 and/or of EMRs/EHRs 172 to train ML algorithm 114. For example, the one or more data sets may be data from individuals with concussions, their treatments, injuries, scans, symptoms, recovery, and the like. Then, data from the multiple individuals (e.g., patients 105) may be used to train ML algorithm 114. Then, healthcare system 100 and/or brain healthcare application 110 may use ML algorithm 114, which has been trained using machine learning techniques, to make certain predictions about a subject (e.g., patients 105). The prediction might be things such as, but not limited to, the probability that the patient will have persistent symptoms, the probability that a particular treatment will be effective, the timelines for recovery, a ranking of treatment effectiveness probabilities, and the like. An output of healthcare system 100 and/or brain healthcare application 110 may include, for example, a report produced using ML algorithm 114 and reporting the predictions. Accordingly, healthcare providers 170 may use the report to aid in developing a treatment plan for a patient 105.

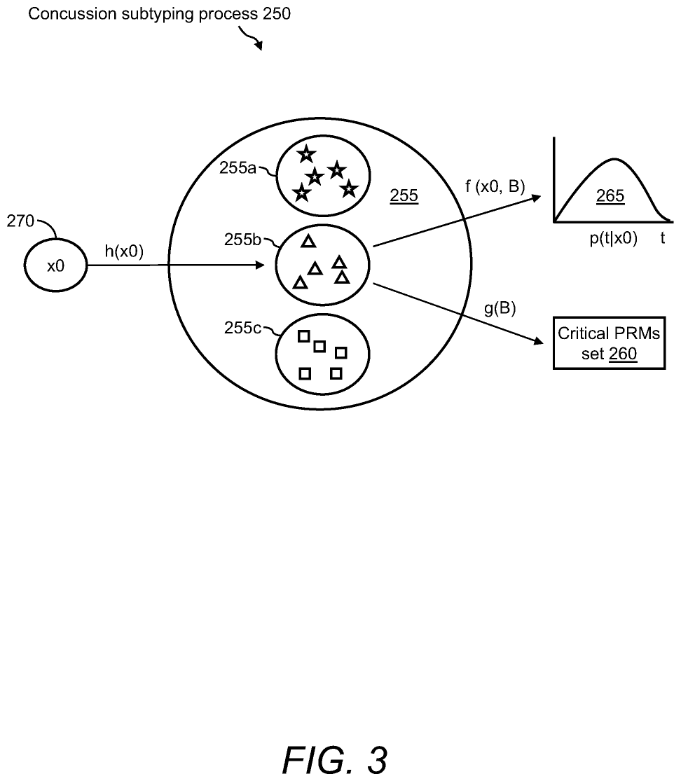

The ML algorithm can be trained using a dataset comprising attributes of reference subjects. The attributes of the reference subjects can be or be based on data from previous patients, current patients, or both. The dataset for training can be received, e.g., curated or in unstructured form from a public database, private database, hospitals, etc. The dataset for training can be crafted using the previous or current patient data by the user (e.g., the patient or a medical professional). The dataset can also comprise clinical outcomes for the reference subjects that were afflicted with a traumatic brain injury. For instance, the clinical outcomes can comprise a recovery phenotype, treatment received (if any), efficacy of the treatment, timeline for recovery, and the like. The machine learning model can be used to process the dataset using the machine learning model, which can generate outputs (e.g., numerical values). The parameters of the machine learning model can be updated optimizing a loss function (e.g., through gradient descent or other appropriate methods for updating the parameters of the machine learning model). The loss function can be based on the clinical outcomes of the reference subjects and the outputs of the machine learning model. When sufficiently trained, the outputs of the machine learning model can be indicative of a recovery phenotype of the traumatic brain injury for the plurality of reference subjects. For instance, FIG. 3 schematically illustrates a representation of a trained machine learning algorithm. The machine learning algorithm can receive the attributes of subjects (“x0”; 270) as input. The machine learning algorithm can “organize” or “sort” the attributes to groups of subjects (stars, triangles, and squares; 255a, 255b, and 255c, respectively) having some similarities or shared attributes. The subjects in a group can be similar in some measure of the outcomes of the subjects, e.g., a recovery phenotype of the subjects. The subjects in a group can have shared attributes that are associated with the measure of the outcome. For instance, the subjects in a group may have a certain combination of attributes that increase the likelihood that the subject has a certain recovery phenotype. In some instances, the combination of attributes may be a combination of old age and the type of event (e.g., car accident) that caused the injury. In some instances, the combination of attributes may be a combination of certain symptoms and a history of concussions for a patient. The machine learning algorithm, through training, can learn to distinguish or to group certain types of subjects together based on important pieces of information provided in the attributes.

The different groups can be more apparent, when for example, a dimensionality reduction technique is used. A dimensionality technique could comprise principle-component analysis, non-linear manifold learning methods such as isomap embedding, spectral embedding, T-distributed stochastic neighboring embedding, an autoencoder, or a combination thereof. An autoencoder architecture can process the attributes to encode salient information in the attributes that provide important information for distinguishing between different patients by a clinical outcome. The encoded latent representations can encode salient information for groups or clusters of subjects that have some similarity based on the outcome and the attributes.

The machine learning algorithm can be used to generate predictions for new patients, or new medical events for previous patients. The machine learning model can be applied to predict a clinical outcome comprising a traumatic brain injury for a patient. The machine learning model can be applied to predict a plurality of treatment options for treatment of the traumatic brain injury for a patient. The machine learning model can be applied to predict a recovery timeline for a patient. The recovery timeline can comprise a timeline of one or more symptoms. For any one of the predictions, the machine learning model can be applied to generate an uncertainty value, a confidence interval, or any measure of statistical uncertainty or confidence associated with the prediction.

While some concussed people (e.g., about 75%) may recover within about one month, some concussed people may experience Persistent Concussion Symptoms (PCS) or Post-Concussion Syndrome (PCS) or Persistent Post-Concussive Symptoms (PPCS). PCS and/or PPCS may refer, for example, to concussion symptoms that may linger for many months or beyond a year. These lingering symptoms may include, for example, headache, neck pain, vision issues, balance issues, sleeping issues, neural cognitive issues, behavioral symptoms, physiologic symptoms, and the like. According, there may be great benefit to identifying early those concussed patients 105 that may be likely to experience PCS and/or PPCS and then treating for PCS and/or PPCS as early as possible. In one example, PCS prediction algorithm 116 may utilize independently machine learning processes as described hereinabove to determine, for example, a % probability of PCS for concussed patients 105. In another example, PCS prediction algorithm 116 together with ML algorithm 114 may utilize machine learning processes to determine, for example, a % probability of PCS for concussed patients 105.

Accordingly, machine learning processes of PCS prediction algorithm 116 (or together with ML algorithm 114) may use one or more data sets to make certain predictions. For example, data sets in user health data 134, brain health intake protocols 140, brain treatment protocols 142, and past cases data 144 at data store 130 and/or of EMRs/EHRs 172 to train ML algorithm 114. For example, the one or more data sets may be data from individuals with PCS. Then, data from the multiple individuals (e.g., patients 105) may be used to train PCS prediction algorithm 116.

For example, there can be certain risk factors that may be predictors of the likelihood of a concussed patient 105 to experience PCS. The PCS predictor risk factors may include, but are not limited to, age, gender, extracranial injury, amnesia, personal history of psychiatric disorder, family history of psychiatric disorder, cause of injury (sports or other), type of sport, personal history of migraines, family history of migraines, number of previous concussions, prior mTBI, depression, anxiety, attention deficit hyperactivity disorder (ADHD), attention-deficit disorder (ADD), personal history of PCS, family history of PCS, stressful life events, income, analgesic before injury, received analgesic in emergency department (ED), mood disorder, number of symptoms, and the like.

Accordingly, risk factors that PCS prediction algorithm 116 may use to predict PCS may comprise, for example, age, gender, extracranial injury, amnesia, personal history of psychiatric disorder, family history of psychiatric disorder, cause of injury (sports or other), type of sport, personal history of migraines, family history of migraines, number of previous concussions, prior mTBI, depression, anxiety, ADHD, ADD, personal history of PCS, family history of PCS, stressful life events, income, analgesic before injury, received analgesic in ED, mood disorder, number of symptoms, and the like.

In one example, PCS prediction algorithm 116 (or together with ML algorithm 114) may be used to aggregate and process the predictor risk factors and then return a % probability of a concussed patient 105 to experience PCS. Determining a high % probability early on may be greatly beneficial to prompt early treatment for the patient 105. Thereby reducing and/or entirely avoiding the onset of PCS and also reducing and/or entirely avoiding certain inconveniences and/or expenses. Healthcare system 100 and/or PCS prediction algorithm 116 are not limited to the PCS predictor risk factors mentioned hereinabove. These are exemplary only. Other PCS predictor risk factors are possible. An output of PCS prediction algorithm 116 (or together with ML algorithm 114) may include, for example, a report of a % probability of PCS for the patient 105. Accordingly, healthcare providers 170 may use the report to aid in developing a treatment plan for patients 105 that have a strong probability of experiencing PCS.

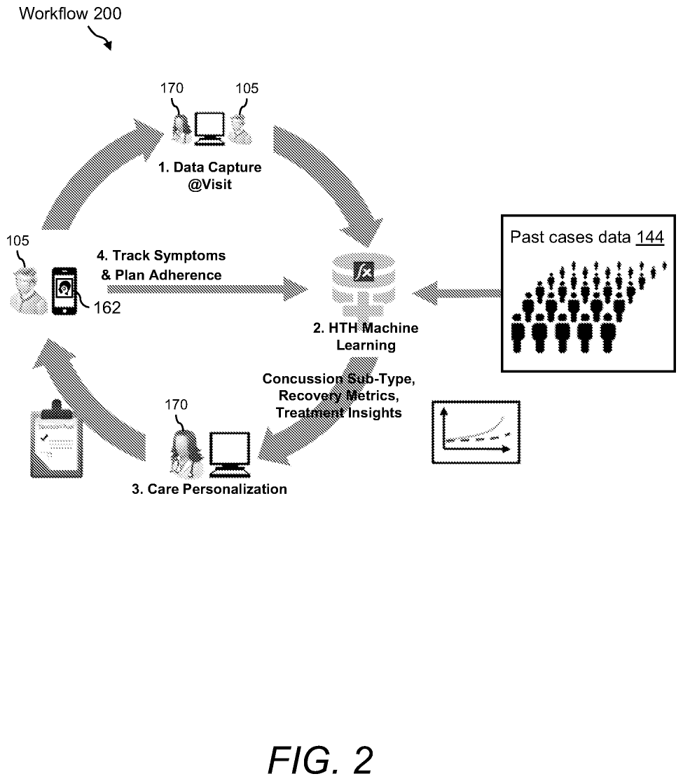

Brain healthcare algorithm 112, ML algorithm 114, PCS prediction algorithm 116, clustering algorithm 118, and/or any other algorithms may be used to classify patient recovery phenotypes (PRP). PRPs may be determined based on symptom and recovery attributes. In one example, a clustering approach may be used to identify PRPs based on, for example, demographic features, injury mechanisms, and recovery paths and then create critical predicted recovery metrics (PRMs) and custom recovery paths. Examples of PRPs may include, but are not limited to, 1) a short recovery female sport related concussion and 2) a long recovery male adult motor vehicle crash. More details of an example of a clustering process are shown and described hereinbelow with reference to FIG. 2 and FIG. 3.

Brain healthcare application 110 of healthcare system 100 feature an easy-to-use communication platform for the interdisciplinary healthcare team and patient. For example, brain healthcare application 110 provides clinician web portal 120, which may be a concussion-specific user interface for healthcare providers 170. Additionally, brain healthcare application 110 provides brain health mobile app 162, which may be a concussion-specific user interfaces for patients 105. Together, clinician web portal 120 and brain health mobile app 162 may be used to structure data intake and convey insights. Example screenshots of clinician web portal 120 are shown hereinbelow with reference to FIG. 8 through FIG. 30. Example screenshots of brain health mobile app 162 are shown hereinbelow with reference to FIG. 31 through FIG. 52.

Additionally, brain healthcare application 110 of healthcare system 100 feature purpose-built machine learning for treatment insights. For example, using the structured data-intake of clinician web portal 120 and/or brain health mobile app 162, machine learning models may be trained to generate phenotypes based on patient and injury characteristics and symptoms across eight domains described above. These phenotypes may then be used to generate the individual recovery probability distributions over time for symptoms in the PRM set across the same eight domains and for the concussion episode overall. The healthcare provider 170 has the option of sharing the recovery probability paths with patients through brain health mobile app 162. Brain health mobile app 162 may be used by the patient throughout the recovery period, allowing the healthcare provider 170 access to detailed and accurate longitudinal patient-reported symptoms.

In some aspects, the present disclosure provides platform for managing brain injuries or concussions. The platform can comprise a client device comprising at least one processor, an operating system configured to perform executable instructions, a memory, and a computer program including instructions executable by the client device to create an application comprising a software module. The software module can be for receiving a plurality of attributes of a subject, wherein the plurality of attributes is related to a traumatic brain injury of the subject. The platform can comprise a server comprising at least one processor, an operating system configured to perform executable instructions, a memory, and a computer program including instructions executable by the client device to create an application comprising a software module. The software module can be for applying a machine learning model to the plurality of attributes to predict (i) a clinical outcome comprising a traumatic brain injury, (ii) a recovery phenotype of the traumatic brain injury, (iii) a plurality of treatment options for treatment of the traumatic brain injury, or (iv) any one of the clinically useful predictions disclosed elsewhere in the application.

In some aspects, the present disclosure provides a computer-implemented system comprising a computing device comprising at least one processor, an operating system configured to perform executable instructions, a memory, and a computer program including instructions executable by the computing device to create an application. The application can comprise a software module configured to receive a plurality of attributes of the subject, wherein the plurality of attributes is related to a traumatic brain injury. The application can comprise a software module configured to apply a machine learning model to the plurality of attributes to predict (i) a clinical outcome comprising a traumatic brain injury, (ii) a recovery phenotype of the traumatic brain injury, (iii) a plurality of treatment options for treatment of the traumatic brain injury, or (iv) any one of the clinically useful predictions disclosed elsewhere in the application.

In some aspects, the present disclosure provides a non-transitory computer-readable storage media encoded with a computer program including instructions executable by one or more processors to generate a selection of treatment options for a subject. The executable instructions can comprise a database manager, in a computer memory, the database of the database manager comprising a plurality of attributes of the subject, wherein the plurality of attributes is related to a traumatic brain injury. The executable instructions can comprise a software module configured to apply a machine learning model to the plurality of attributes to predict (i) a clinical outcome comprising a traumatic brain injury, (ii) a recovery phenotype of the traumatic brain injury, (iii) a plurality of treatment options for treatment of the traumatic brain injury, or (iv) any one of the clinically useful predictions disclosed elsewhere in the application.

FIG. 2 shows a flow diagram of an example of a workflow 200 of the healthcare system 100 for managing brain injury or concussion. Using brain healthcare application 110 of healthcare system 100, the workflow 200 can derive a concussion subtype, and recovery metrics for that subtype, and treatment insights. Treatment insights may include, but are not limited to, treatment activities, medications, referrals to specialists, any necessary restrictions for given symptoms, and the like.

For example, a step #1 may be a data capture step. The data capture step may start after a diagnosis of a concussion has occurred. In this step, brain healthcare application 110 can provide a clinically customized structured data intake process for the clinician (e.g., healthcare provider 170) and by which the clinician may collect information from the patient 105 seeking treatment for the possibility of mTBI or concussion.

Next, at a step #2, the information collected in the data capture step may be stored in data store 130, which can include the machine learning database that may be informed by past cases data 144. The information in past cases data 144 may originate from multiple internal and/or external sources (e.g., public information, EMRs/EHRs 172, and the like). Further, past cases data 144 may include treatment insights from past concussion cases, as more clinics use brain healthcare application 110 of healthcare system 100 more and more information may be included in past cases data 144. In this step, using brain healthcare algorithm 112, ML algorithm 114, PCS prediction algorithm 116, and/or clustering algorithm 118, machine learning may be applied to process the intake information of the patient 105 of interest along with information in past cases data 144 and/or EMRs/EHRs 172.

Next, at a step #3, a personalized treatment plan may be developed by the clinician. For example, the clinician (e.g., healthcare provider 170) has access to information in past cases data 144 at data store 130 (the machine learning database). This information may include insights that the clinician can leverage in the recommended treatment plan to their patients. For example, brain healthcare algorithm 112, ML algorithm 114, PCS prediction algorithm 116, and/or clustering algorithm 118 may use data from past cases data 144 to derive insights that are inclusive of the patent's demographics, injury characteristics, the clinical domains that are relevant from previous cases, previous recoveries, and how they were treated—this is all input to how brain healthcare application 110 derives a concussion subtype for a new patient and suggests a certain brain treatment protocol 142.

For example, FIG. 3 shows a schematic diagram of an example of a concussion subtyping process 250. In brain healthcare application 110 of healthcare system 100, clustering algorithms (e.g., clustering algorithm 118) may be used to determine PRPs. The clustering algorithms (e.g., clustering algorithm 118) may use an appropriate distance metric to group patient episodes with similar symptom and recovery attributes. Herein, distance refers to the dissimilarity between two patients in a high-dimensional space. The data used for the clustering algorithm may, for example, be information from the initial post-injury clinical evaluation and from subsequent clinical evaluations symptom-monitoring during the recovery period. Then, critical PRMs and individual probabilistic recovery paths may be generated from the structured data-intake of brain healthcare application 110.

In one example, concussion subtyping process 250 may be performed using brain healthcare algorithm 112, ML algorithm 114, PCS prediction algorithm 116, and/or clustering algorithm 118 of brain healthcare application 110. First, subtype groups 255 are established. For example, subtype groups 255 may include subtype groups 255a, 255b, 255c, and so on. Next, each of the subtype groups 255 (e.g., subtype groups 255a, 255b, 255c) may be mapped to a critical PRMs set (g(B)) 260 and to a probabilistic recovery curve (p(t|x0) 265. For each patient subtype group (255), an individual's probabilistic recovery curve 265 may be calculated in a manner conditional on their time-0 attributes and to identify critical PRMs set 260. Next, any new concussion patient 270 may be mapped to a certain subtype group (h(x0)) 255.

Each of the subtype groups 255 (e.g., subtype groups 255a, 255b, 255c) may include, for example, similar cases according to personal characteristics and patient recovery phenotypes (PRPs). That is, each of the subtype groups 255 may represent a certain concussion subtype. For example, subtype group 255a may represent one concussion subtype. Subtype group 255b may represent another concussion subtype. Subtype group 255c may represent yet another concussion subtype.

Further, the subtype groups 255 (or concussion subtypes) may be based on machine learning. For example, ML may be used to group data into similarity groups based on patient segments, injuries, and recovery metrics using, for example, clustering algorithm 118. For example, machine learning of brain healthcare algorithm 112, ML algorithm 114, PCS prediction algorithm 116, and/or clustering algorithm 118 of brain healthcare application 110. Accordingly, a new concussion patient 270 may be assigned into a specific subtype group 255 of similar cases according to his/her personal characteristics and similar PRPs. Further, critical PRMs set 260 may be associated with any phenotype. Further, any initial symptom and for the episode overall may have a predicted recovery probability path over time.

Examples of subtype groups 255 or concussion subtypes may include, but are not limited to, 1) a short recovery female sport related concussion and 2) a long recovery male adult motor vehicle crash.

Referring to step #3 of workflow 200 shown in FIG. 2, once the concussion subtype (or brain treatment protocol 142) is determined, based on that concussion subtype, brain healthcare application 110 may recommend the brain treatment protocol 142 that includes the treatment insights and/or plans that are likely to drive the patient's optimal recovery path.

For example, once a new patient 105 is classified into a recovery phenotype cohort, the patient 105 may inherit the associated critical PRM set and predicted recovery identified through the machine learning models (e.g., of healthcare algorithm 112, ML algorithm 114, PCS prediction algorithm 116, and/or clustering algorithm 118 of brain healthcare application 110). Once a patient 105 is assigned to a patient subtype group (255), the healthcare provider 170 may see the patient 105's PRMs and predicted recovery path and can then use it when deciding how to treat the patient.

Further, brain healthcare algorithm 112, ML algorithm 114, PCS prediction algorithm 116, and/or clustering algorithm 118 may include a certain match threshold that causes a patient 105 to fit into a certain concussion subtype. However, in the case in which a patient 105 may not meet the threshold of any particular concussion subtype, then brain healthcare algorithm 112, ML algorithm 114, PCS prediction algorithm 116, and/or clustering algorithm 118 may suggest a best treatment match even if not highly optimal.

Next, a step #4 may be a treatment protocol step for tracking symptoms of the patient 105 and monitoring treatment plan adherence. This step may occur between visits with the healthcare provider 170 and may rely on the patient 105 using brain health mobile app 162. For example, brain health mobile app 162 may be used to provide symptom updates to the clinician. Further, brain health mobile app 162 may be used to indicate to the clinician whether the patient 105 is adhering to the treatment plan. Throughout the steps of workflow 200 of the healthcare system 100, information in data store 130 may be updating continuously. Accordingly, in this step, the healthcare provider 170 can monitor patient information and even intervene if necessary.

The steps of workflow 200 may repeat with each visit. Concussion patients may have three or more visits with their healthcare provider 170. Further, in workflow 200, brain healthcare algorithm 112, ML algorithm 114, PCS prediction algorithm 116, and/or clustering algorithm 118 may be used to continuously processes patent data and adjust treatment plans accordingly over time. For example, it may be possible that the concussion subtype might change over time.

Referring to FIG. 1, FIG. 2, and FIG. 3, the healthcare system 100 may provide a digital health platform that may be used to capture and leverage clinically customized structured data to enable ML treatment insights from real-world concussion patient data to guide personalized care. Healthcare system 100 including brain healthcare application 110 may provide a personalized treatment care plan for individuals experiencing concussion to ultimately improve patient outcomes. Healthcare system 100 may provide a comprehensive and well-coordinated means for extracting critical information about treatment and recovery from a growing set of available but disparate data. Making this possible in healthcare system 100 is robust data collection and synchronization schemes that feed, for example, brain healthcare algorithm 112, ML algorithm 114, PCS prediction algorithm 116, and/or clustering algorithm 118. This also enables clinicians to leverage the combined lessons from real-world patients to personalize a treatment regimen that can be confidently tracked and adapted in real-time. For patients 105, this personalized care can result in faster and more reliable outcomes. For healthcare providers 170, more consistent visibility to the critical health metrics associated with each patient leads to more confident and effective decisions about patient care. At the population level, healthcare system 100 may provide means for highly data-driven protocols and may also reduce concussion-related healthcare costs.

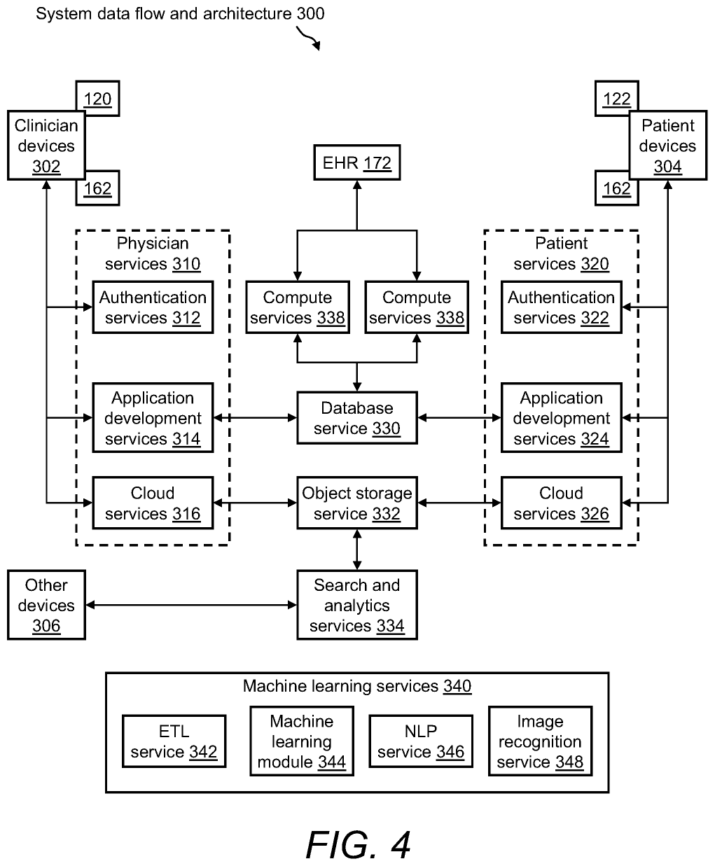

FIG. 4 shows a block diagram of an example of a system data flow and architecture 300 of the healthcare system 100 for managing brain injury or concussion. In one example, system data flow and architecture 300 may include a physician services portion, which is physician services 310, and a patient services portion, which is patient services 320.

Physician services 310 may be accessed by healthcare providers 170 using, for example, their clinician devices 302. Physician services 310 may include, for example, authentication services 312, application development services 314, and cloud services 316. Similarly, physician services 310 may be accessed by patients 105 using, for example, their patient devices 304. Patient services 320 may likewise include, for example, authentication services 322, application development services 324, and cloud services 326.

System data flow and architecture 300 can further include a database service 330, an object storage service 332, search and analytics services 334, and compute services 338. Database service 330 can support both application development services 314 of physician services 310 and application development services 324 of patient services 320. Database service 330 can also support EMRs/EHRs 172 via one or more compute services 338. Object storage service 332 can support both cloud services 316 of physician services 310 and cloud services 326 of patient services 320. Further, search and analytics services 334 may be accessed by other devices 306, such as those of system administrators and/or system analysts.

System data flow and architecture 300 can further include certain machine learning services 340. In one example, machine learning services 340 may include an extract, transform, and load (ETL) service 342, a machine learning module 344, a natural-language processing (NLP) service 346, and an image recognition service 348.

In system data flow and architecture 300, authentication services 312, 322 may be used to provide simple and secure user sign-up, sign-in, and access control to web and/or mobile apps. For example, authentication services 312, 322 may support sign-in with social identity providers, such as Apple, Facebook, Google, and Amazon, and enterprise identity providers.

In system data flow and architecture 300, application development services 314, 324 may be used to develop GraphQL APIs and gives front-end developers the ability to query multiple databases, microservices, and APIs with a single GraphQL endpoint.

In system data flow and architecture 300, cloud services 316, 326 may be used to securely deliver content with low latency and high transfer speeds. Cloud services 316, 326 may be, for example, a content delivery network (CDN) service built for high performance, security, and developer convenience.

In system data flow and architecture 300, database service 330 may be, for example, a fast, flexible NoSQL database service. For example, a fully managed, serverless, key-value NoSQL database designed to run high-performance applications at any scale. Features may include, for example, built-in security, continuous backups, automated multi-region replication, in-memory caching, and data export tools.

In system data flow and architecture 300, object storage service 332 can provide, for example, an object storage service with high scalability, data availability, security, and performance. Object storage service 332 may be used to store and protect any amount of data, such as data lakes, cloud-native applications, and mobile apps.

In system data flow and architecture 300, search and analytics services 334 may be used to search, visualize, and analyze up to petabytes of text and unstructured data. Further, search and analytics services 334 may be used to perform interactive log analytics, real-time application monitoring, website search, and the like.

In system data flow and architecture 300, compute services 338 can provide, for example, a serverless, event-driven compute service in which code may be run for any type of application or backend service without provisioning or managing servers.

In system data flow and architecture 300, ETL service 342 of machine learning services 340 may be used to reliably capture, transform, and deliver streaming data to data lakes, data stores, and analytics services. For example, ETL service 342 may be used to stream data into object storage service 332 and convert data into required formats for analysis without building processing pipelines.

In system data flow and architecture 300, machine learning module 344 of machine learning services 340 may be used to build, train, and deploy machine learning (ML) models for any use case with fully managed infrastructure, tools, and workflows.

In system data flow and architecture 300, NLP service 346 of machine learning services 340 may be, for example, an NLP service that uses machine learning to uncover valuable insights and connections from text within documents. For example, NLP service 346 may be used to process text to extract the key phrases, entities, and sentiment for further analysis.

In system data flow and architecture 300, image recognition service 348 of machine learning services 340 may be used, for example, for object and scene detection, facial recognition, facial analysis, face comparison, unsafe (or inappropriate) image detection, and celebrity recognition.

In one example, system data flow and architecture 300 may be implemented using Amazon Web Services (AWS) that may include certain Amazon products. In one example, authentication services 312, 322 may be Amazon Cognito; application development services 314, 324 may be AWS AppSync; cloud services 316, 326 may be Amazon Cloudfront; database service 330 may be Amazon DynamoDB; object storage service 332 may be Amazon Simple Storage Service (Amazon S3); search and analytics services 334 may be Amazon OpenSearch Service; compute services 338 may be AWS Lambda; ETL service 342 may be Amazon Kinesis Data Firehose; machine learning module 344 may be Amazon SageMaker; NLP service 346 may be Amazon Comprehend; and image recognition service 348 may be Amazon Rekognition Image.

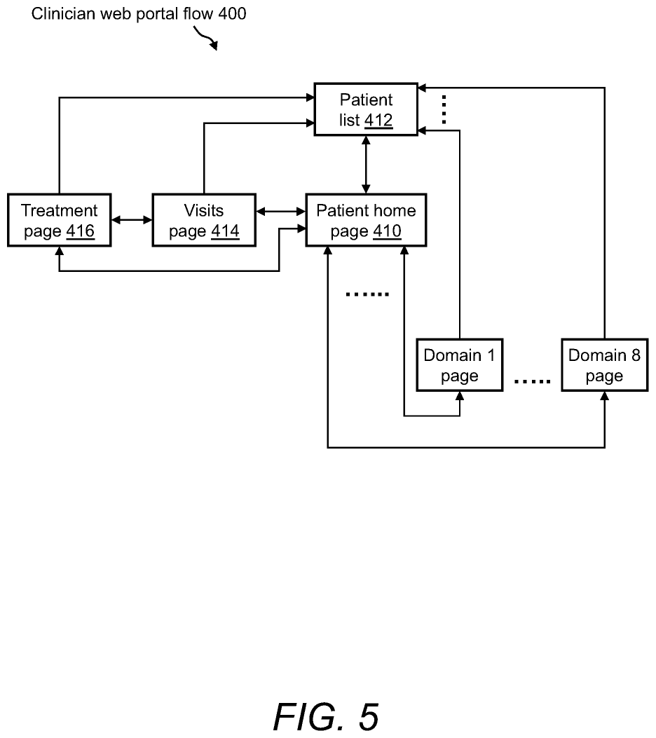

FIG. 5 shows a flow diagram of an example of a clinician web portal flow 400 of the healthcare system 100 for managing brain injury or concussion. For example, clinician web portal flow 400 may show the workflow of clinician web portal 120 of brain healthcare application 110. For example, clinician web portal flow 400 may include a patient home page 410, a patient list 412, a visits page 414, a treatment page 416, and multiple domain pages (i.e., clinical domain pages), such as domain 1 through domain 8 pages.

In brain healthcare application 110 there may be eight clinical domains with respect to mild traumatic brain injury (mTBI) or concussion. These eight clinical domains may be, for example, headache, sleep-wake, cervicogenic (i.e., neck), ocular (i.e., vision), vestibular (i.e., balance), behavioral, cognitive, and physiologic. Accordingly, in clinician web portal flow 400, a domain 1 page may be the “headache” domain page. A domain 2 page may be the “sleep-wake” domain page. A domain 3 page may be the “cervicogenic” domain page. A domain 4 page may be the “ocular” domain page. A domain 5 page may be the “vestibular” domain page. A domain 6 page may be the “behavioral” domain page. A domain 7 page may be the “cognitive” domain page. A domain 8 page may be the “physiologic” domain page. However, brain healthcare application 110 is not limited to eight domains only and these particular domains only. Brain healthcare application 110 may include one or more of the aforementioned domains as well as any number of other domains.

In clinician web portal flow 400, the workings of clinician web portal 120 may be based on clinician web portal flow 400. For example, patient home page 410 may be used to navigate to any patient 105 in patient list 412, which may include a record of all patients 105 associated with healthcare system 100. Further, patient home page 410 may be used to navigate to visits page 414, which may include a record of any and all visits by the patient 105 selected from patient list 412. Further, patient home page 410 may be used to navigate to treatment page 416, which may include a record of any and all treatments received by the patient 105 selected from patient list 412. Further, patient home page 410 may be used to navigate to any one of the domain 1 through domain 8 pages that may apply to the patient 105 selected from patient list 412.

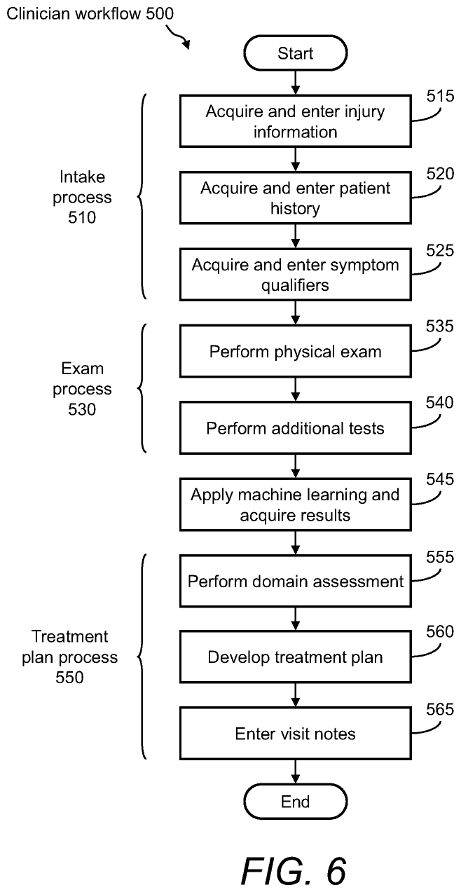

FIG. 6 shows a flow diagram of an example of a clinician workflow 500 of the healthcare system 100 for managing brain injury or concussion. In one example, clinician workflow 500 may include an intake process 510 including a step 515, a step 520, and a step 525. Then, intake process 510 may be followed by an exam process 530 including a step 535 and a step 540. Then, exam process 530 may be followed by a machine learning step 545. Then, machine learning step 545 may be followed by a treatment plan process 550 including a step 555, a step 560, and a step 565. Accordingly, clinician workflow 500 may include, but is not limited to, the following steps.

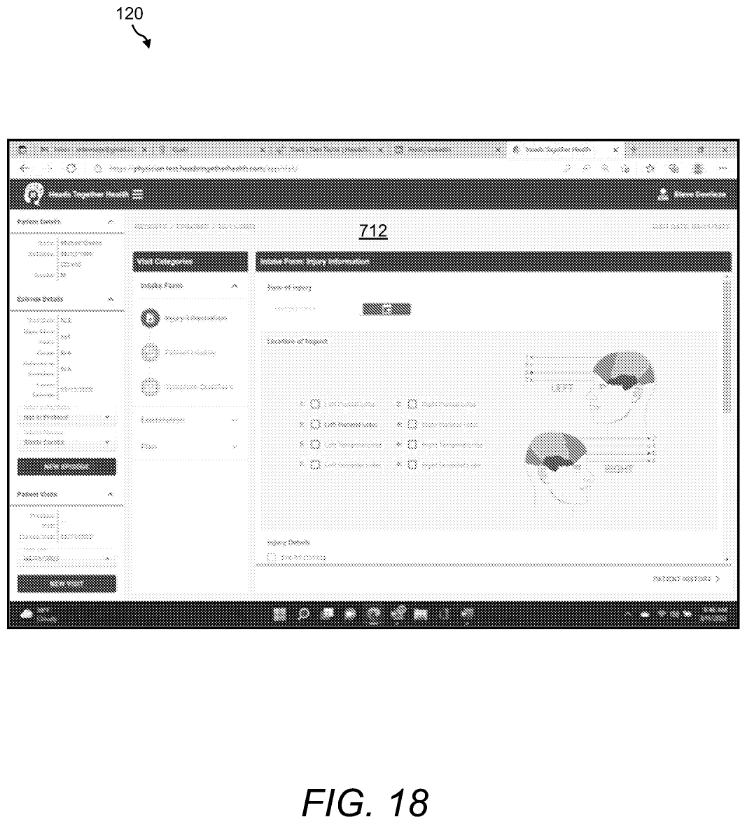

At step 515 of intake process 510, injury information about the patient is acquired and entered. For example, healthcare provider 170 may use clinician web portal 120 of brain healthcare application 110 of healthcare system 100 to acquire and enter injury information about the patient 105 of interest. The source of the injury information may, for example, be the patient 105 himself/herself, information in user health data 134, information in EMRs/EHRs 172, and the like. Examples of data intake screens of clinician web portal 120 showing injury information are shown hereinbelow with reference to FIG. 18.

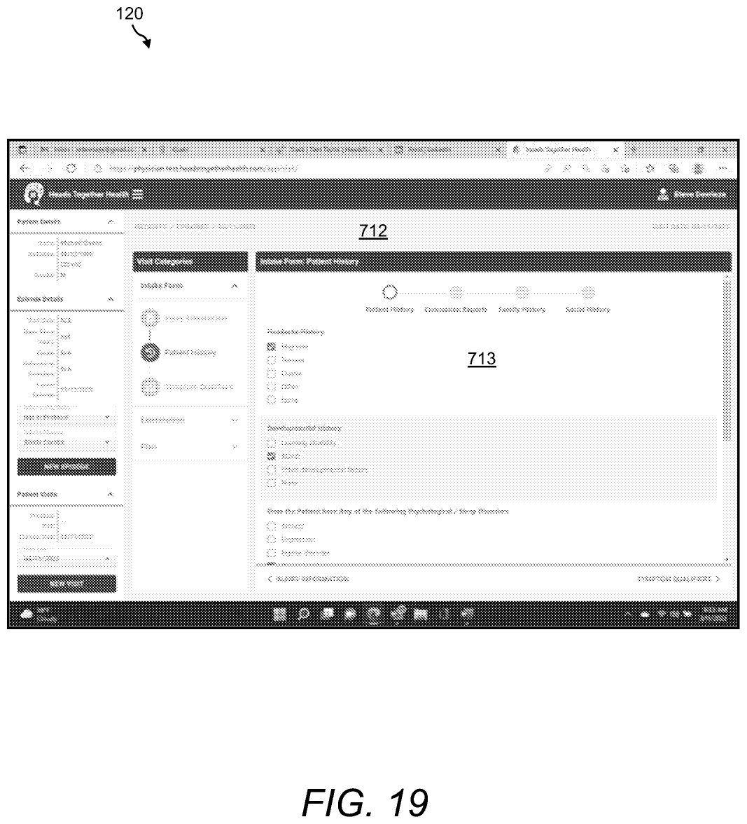

At step 520 of intake process 510, patient history information about the patient can be acquired and entered. For example, healthcare provider 170 may use clinician web portal 120 of brain healthcare application 110 of healthcare system 100 to acquire and enter patient history information about the patient 105 of interest. The source of the patient history information may, for example, be the patient 105 himself/herself, information in user health data 134, information in EMRs/EHRs 172, and the like. Examples of data intake screens of clinician web portal 120 showing patient history information are shown hereinbelow with reference to FIG. 19 through FIG. 22.

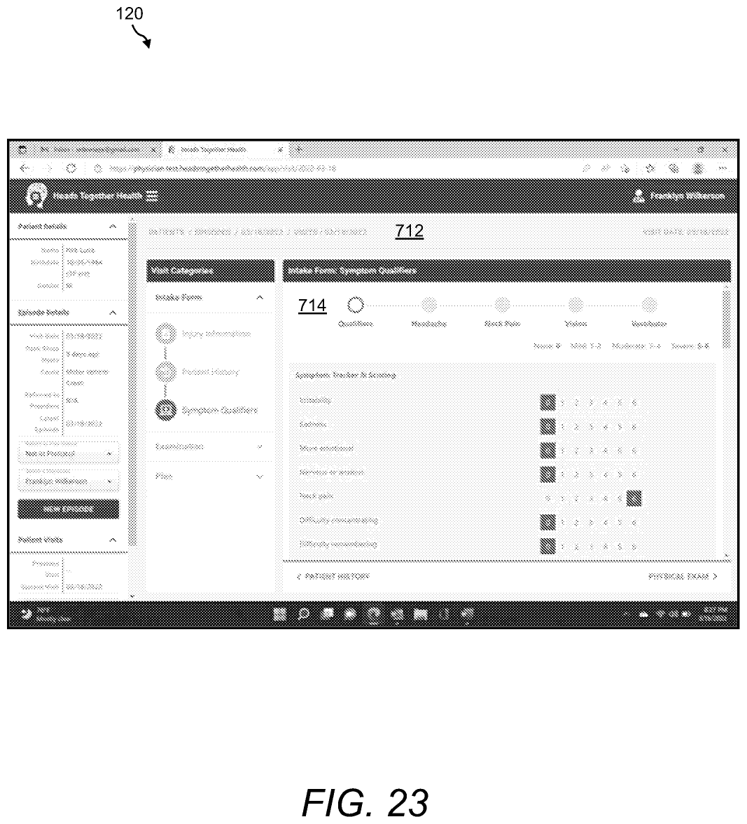

At step 525 of intake process 510, symptom qualifiers information about the patient can be acquired and entered. For example, healthcare provider 170 may use clinician web portal 120 of brain healthcare application 110 of healthcare system 100 to acquire and enter symptom qualifiers information about the patient 105 of interest. The source of the symptom qualifiers information may, for example, be the patient 105 himself/herself, information in user health data 134, information in EMRs/EHRs 172, and the like. Examples of data intake screens of clinician web portal 120 showing symptom qualifiers are shown hereinbelow with reference to FIG. 23. Symptom qualifiers may be any information that describes the presence, absence, state, and/or condition of any symptom, for example, “headache=6” or “dizziness=2”, with respect to mTBI or concussion.

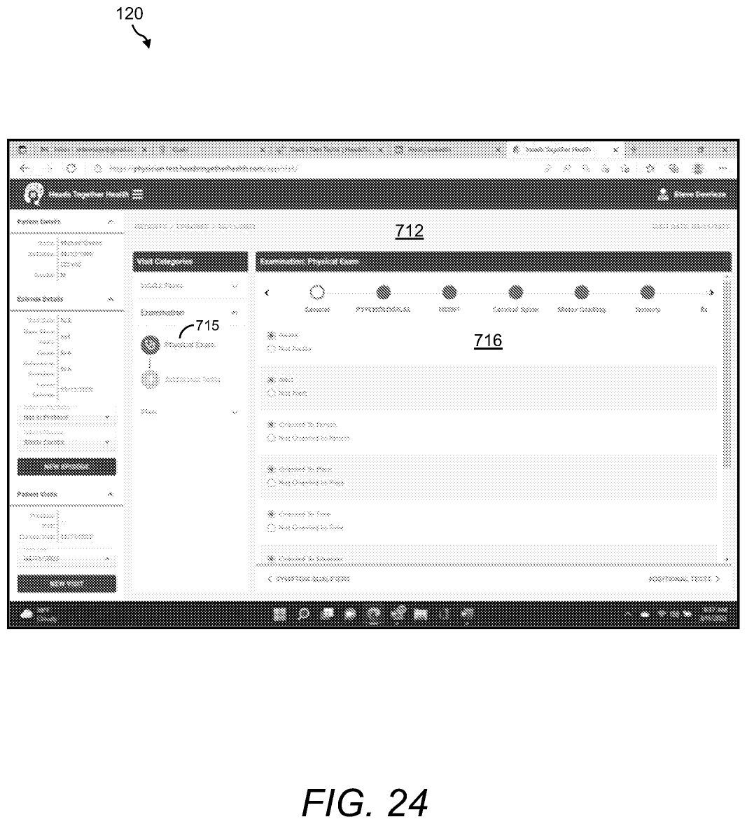

At step 535 of exam process 530, a physical exam of the patient can be performed. For example, healthcare provider 170 performs a physical examination of the patient 105 of interest. Examples of data intake screens of clinician web portal 120 showing physical exam information are shown hereinbelow with reference to FIG. 24.

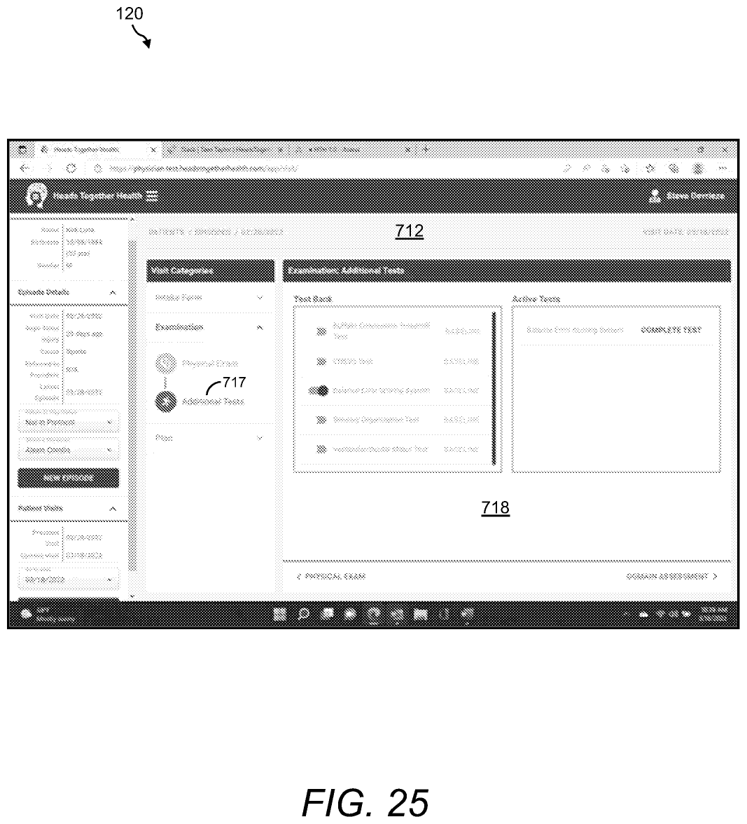

At step 540 of exam process 530, additional tests of the patient can be performed. For example, healthcare provider 170 may order the patient 105 of interest to undergo other tests beyond the physical exam, such as blood tests, cognitive texts, and the like. Examples of data intake screens of clinician web portal 120 showing additional tests information are shown hereinbelow with reference to FIG. 25.

At machine learning step 545, machine learning can be applied and the results can be acquired. For example, machine learning may be applied per ML algorithm 114 and/or PCS prediction algorithm 116 of brain healthcare application 110. That is, the machine learning processes may use one or more data sets in user health data 134, brain health intake protocols 140, brain treatment protocols 142, and past cases data 144 at data store 130 and/or of EMRs/EHRs 172 to train of ML algorithm 114 and/or PCS prediction algorithm 116 to make certain predictions. For example, the one or more data sets may be data from individuals with concussions, their treatments, injuries, scans, symptoms, recovery, and the like. Also, data from the multiple individuals (e.g., patients 105) may be used to train ML algorithm 114 and/or PCS prediction algorithm 116. Then, healthcare system 100 and/or brain healthcare application 110 may use ML algorithm 114 and/or PCS prediction algorithm 116, which have been trained using machine learning techniques, to make certain predictions about a subject (e.g., patients 105). The prediction might be things such as, but not limited to, the probability that the patient will have PCS, the probability that a particular treatment will be effective, a ranking of treatment effectiveness probabilities, and the like. An output of healthcare system 100 and/or brain healthcare application 110 may include, for example, a report produced using ML algorithm 114 and/or PCS prediction algorithm 116 and reporting the predictions. Accordingly, healthcare providers 170 may use the report to aid in developing a treatment plan for a patient 105.

At step 555 of treatment plan process 550, a domain assessment can be performed. For example, using brain healthcare algorithm 112, ML algorithm 114, PCS prediction algorithm 116, and/or clustering algorithm 118 of brain healthcare application 110 the patient information may be processed and an assessment be done with respect to the presence, absence, and/or degree of one or more clinical domains with respect to mTBI or concussion. The clinical domains may be, for example, headache, sleep-wake, cervicogenic (i.e., neck), ocular (i.e., vision), vestibular (i.e., balance), behavioral, cognitive, and physiologic. Examples of data intake screens of clinician web portal 120 showing domain assessment information are shown hereinbelow with reference to FIG. 18 through FIG. 29.

At step 560 of treatment plan process 550, a treatment plan can be developed for the patient. For example, assisted by information in the reports generated by brain healthcare algorithm 112, ML algorithm 114, PCS prediction algorithm 116, and/or clustering algorithm 118 of brain healthcare application 110, the healthcare provider 170 may develop a treatment plan for a patient 105. For example, the reports may direct the healthcare provider 170 to a certain treatment plan in brain treatment protocols 142 at data store 130. Examples of data intake screens of clinician web portal 120 showing treatment plans are shown hereinbelow with reference to FIG. 18 through FIG. 29.

At step 565 of treatment plan process 550, visit notes can be entered by the clinician and logged into the system. For example, using clinician web portal 120, the healthcare provider 170 may enter any visit notes, which may be logged in the user health data 134 at data store 130 for the patient 105 of interest. Examples of data intake screens of clinician web portal 120 showing visit notes are shown hereinbelow with reference to FIG. 18 through FIG. 29.

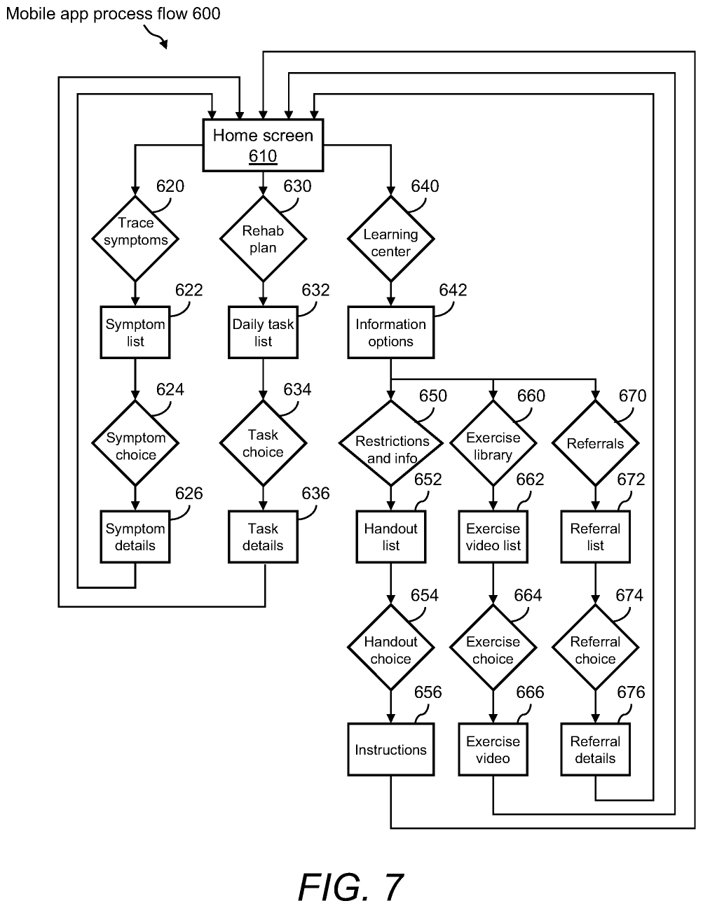

FIG. 7 shows a flow diagram of an example of a mobile app process flow 600 of the healthcare system 100 for managing brain injury or concussion. For example, mobile app process flow 600 may show the workflow of brain health mobile app 162 of brain healthcare application 110. For example, a home screen 610 of brain health mobile app 162 may direct to three paths: (1) a trace symptoms path 620, (2) a rehab plan path 630, and (3) a learning center path 640.

Selecting the trace symptoms path 620 from home screen 610, a symptom list 622 may be displayed to the user. Then, the user may make a symptom choice 624 from the symptom list 622. Then, symptom details 626 of the selected symptom may be displayed to the user.

Selecting the rehab plan path 630 from home screen 610, a daily task list 632 may be displayed to the user. Then, the user may make a task choice 634 from the daily task list 632. Then, task details 636 of the selected task may be displayed to the user.

Selecting the learning center path 640 from home screen 610, information options 642 may be displayed to the user. For example, the information options 642 may include restrictions and information 650, an exercise library 660, and referrals 670.

Then, from restrictions and information 650, a handout list 652 may be displayed to the user. Then, the user may make a handout choice 654 from the handout list 652. Then, instructions 656 about the selected handout may be displayed to the user.

Then, from exercise library 660, an exercise video list 662 may be displayed to the user. Then, the user may make an exercise choice 664 from the exercise video list 662. Then, an exercise video 666 about the selected exercise may be displayed to the user.

Then, from referrals 670, a referral list 672 may be displayed to the user. Then, the user may make a referral choice 674 from the referral list 672. Then, referral details 676 about the selected referral may be displayed to the user.

Below is an outline of an example of using clinician web portal 120 (or patient dashboard 120) and/or brain health mobile app 162. Further, FIG. 8 through FIG. 54 is an example of a process of using clinician web portal 120 (or patient dashboard 120) and/or brain health mobile app 162 of brain healthcare application 110 of the healthcare system 100 for managing brain injury or concussion.
1. Clinician Web Portal
   a. Login
   b. Patient Dashboard
      i. Patient Segments and Verbiage
      ii. Data Presented and metrics provided
   c. Patient Detailed View
      i. Summary Demographic and Status Information
      ii. Drilling into Symptom Trend and Adherence Charts (summary)
         1. Drilling further into Clinical Domains—symptom level trend details
      iii. View latest symptom tracking report
   d. Adding a new patient concussion—Inactive Patients/Create Episode/Create Visit
      i. Select patient from Inactive List
      ii. Select Physician and date from drop down list
      iii. New Episode Screen pops-up (with blank data)
      iv. Create New Visit
      v. Select Visit Date and Submit
   e. Data Capture for a New Visit
      i. Intake Form
         1. Injury Information
         2. Patient History
         3. Symptom Qualifiers
      ii. Examination
         1. Physical Exam
         2. Tests
      iii. Plan
         1. Domain Assessment
         2. Treatment Plan
         3. Visit Note
         4. Copy Visit Note to EMR
         5. Complete Visit
2. Patient Mobile App
   a. Retrieve and install mobile app
   b. Login
   c. Symptom Tracker
   d. Treatment Plan—Record Adherence
3. Clinician Web Portal
   a. Track progress of patient in between visits
      i. Symptom Charts (overall and detailed)

Clinician web portal 120 (aka patient dashboard 120) may be a custom interface that may be used by healthcare providers 170. The functionality of clinician web portal 120 (aka patient dashboard 120) may provide the ability to, for example,
(1) Track summary of patients under care—active/inactive/cleared, name, time since injury, treatment plan adherence, symptom burden and recent trend, upcoming referral activities, next appointment and recent app usage; and
(2) Manage the full treatment continuum coverage from patient information through to treatment plan and visit notes. For example, data and notes captured using clinician web portal 120 may be integrated with EMR (not duplicative with EMR), with a summary automatically feeding EMRs/EHRs 172.

Referring to FIG. 8 through FIG. 30, following login using a standard login screen (not shown), a home page of clinician web portal 120 may be displayed to the healthcare provider 170. An example of a home page 700 of clinician web portal 120 is shown in FIG. 8.

In home page 700, Active Patients can be those undergoing concussion recovery; Inactive Patients can be those who are part of a baseline testing and/or medical history capture (if applicable). Further, Cleared Patients can be those who have completely recovered from a concussion episode. In this example, a test sandbox has three active patients, Kirk Luna, Lucas Morgan, and Marianne Nguyen. The data for Kirk Luna is fairly thorough, though Lucas Morgan and Marianne Nguyen are not strong examples for review based on minimal data capture. Note that there may be some customizations, such as the use of “Players” instead of “Patients.” These nomenclature features may be customized.

Within the Active Players section, there can be a list of patient names, when they sustained their injury (“Date of Injury”), their most recent adherence score (% of rehab activities and medications completed per the mobile app reports), their most recent total symptom burden score, the trend from the previous score, if they have referrals, when is their next appointment scheduled in the clinic, if they have been using the mobile app (most recent date), if they have baseline tests available and there is also a download feature. The download feature may, for example, provide the ability to download and print PDFs of all data captured from visits within a concussion episode.

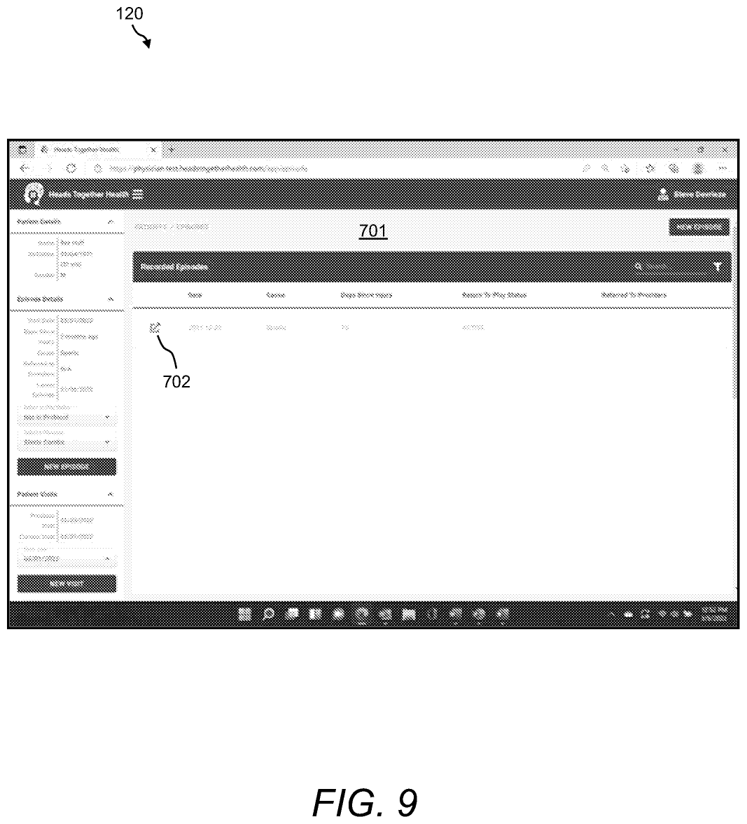

In one example, at home page 700, the healthcare provider 170 may click on the name of an Active Patient within the Active Players list (suggest Kirk Luna). For example, having clicked on Kirk Luna a Patient Detailed View 701 may be displayed, as shown, for example, in FIG. 9. The Patient Detailed View 701 provides Summary Demographics and Status information. Once a patient is selected, more details about that patient's episode and visit status can be shown. For example, the left column shows an overview of the patient information. Healthcare providers 170 may edit return to play status, select a different physician, create a new episode or a new visit, and view a different a visit date. Further, to open a recorded episode, click on the arrow/box button 702 (next to the date of the recorded episode) of Patient Detailed View 701.

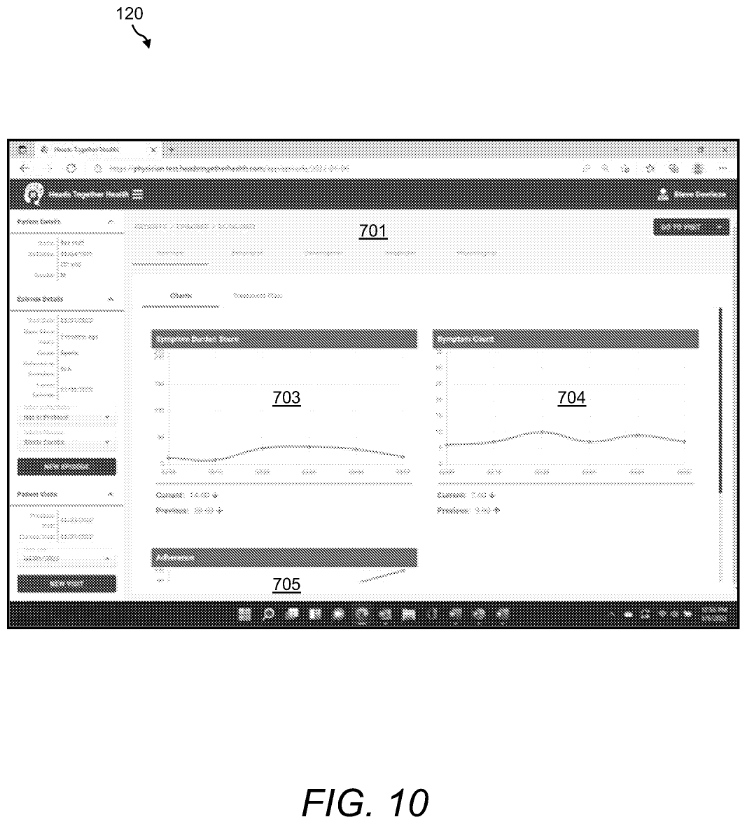

Referring now to FIG. 10, having selected a recorded episode, the Patient Detailed View 701 can display Symptom Trend and Adherence Charts information. This information may include, for example, an overview of the patient's charts based off the recorded data can be observed here. FIG. 10 shows an example of a patient record, where there are many data points over time. In this example for Kirk Luna, there is just two data points. In this example, this data includes the symptom ratings form the date of visit(s), the patient reported symptom and adherence tracking data as reported by the Patient via the brain health mobile app 162, and any symptom tracker tests which were entered directly through the portal. This feature may be beneficial for workflows where there is more frequent engagement between patient and ATC in between visits with the physician (where only the symptom tracker is populated, rather than a full clinical exam). FIG. 10 shows plots 703, 704, and 705. Plot 703 is the total symptom burden score trend. Plot 704 is the symptom count trend. Plot 705 is the treatment adherence trend.

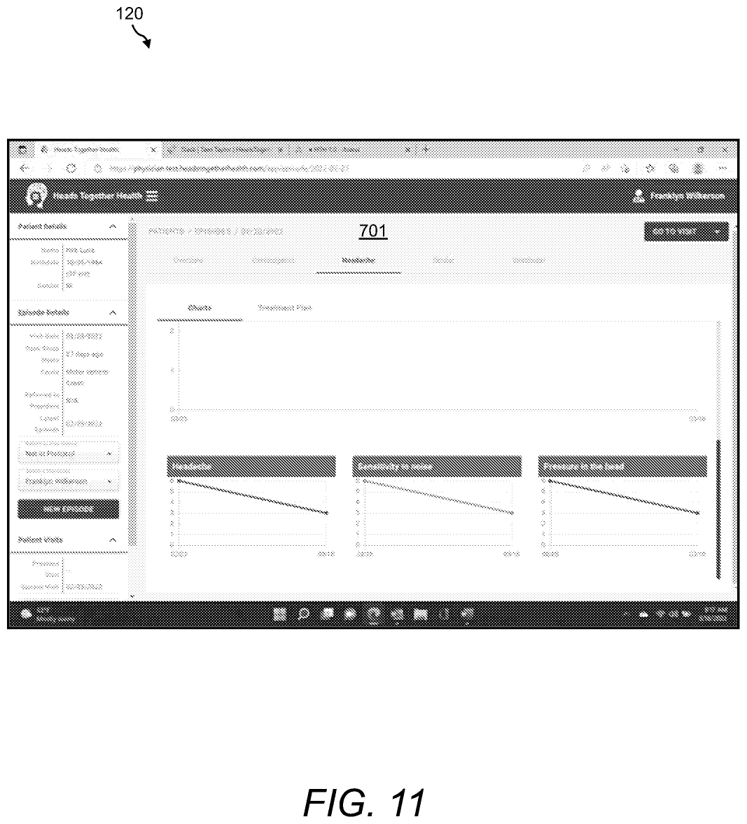

Referring now to FIG. 11, the Patient Detailed View 701 can display Detailed Clinical Domain/Symptom Tracking information. Here, healthcare providers 170 may look at the charts and treatment plans of a specific domain by clicking one of the affected domain tabs. Example domains may include, but are not limited to, Behavioral, Cervicogenic, Cognitive, Headache, Ocular, Physiologic, Sleep-Wake, and Vestibular. For the example of Kirk Luna, domains affected were Cervicogenic, Headache, Ocular and Vestibular. This can vary by patient based on physician selection of domains affected within the visit data capture. The Patient Detailed View 701 of FIG. 11 shows, for example, a specific symptom trend over time.

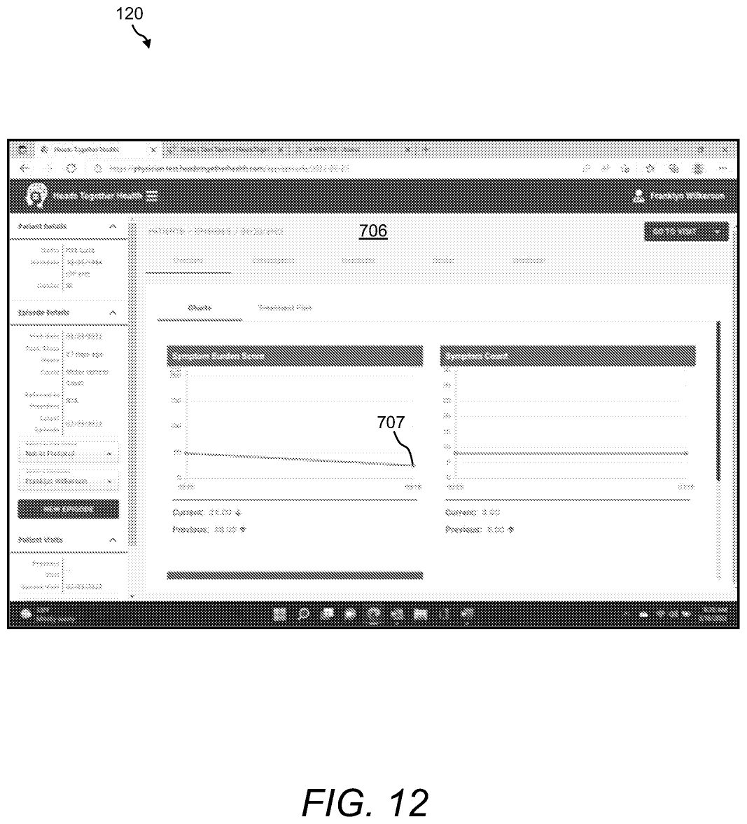

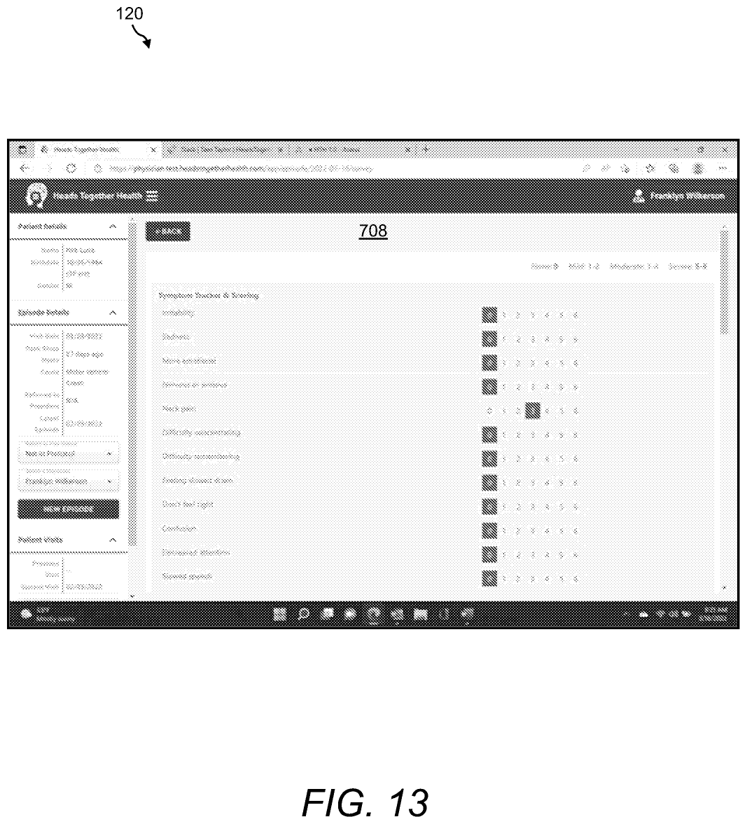

Referring now to FIG. 12, clinician web portal 120 may provide Symptom Tracking Report 706. Here, a top-level symptom tracking chart may be displayed. To see the most recent detailed symptom tracker, a use may click, for example, on a dot 707 from the most recent date. Having clicked on the dot 707, the latest Symptom Tracker Test 708 may be displayed, as shown, for example, in FIG. 13. At FIG. 13, the healthcare provider 170 may click the Back button at the top and then click the Patients link near the top left.

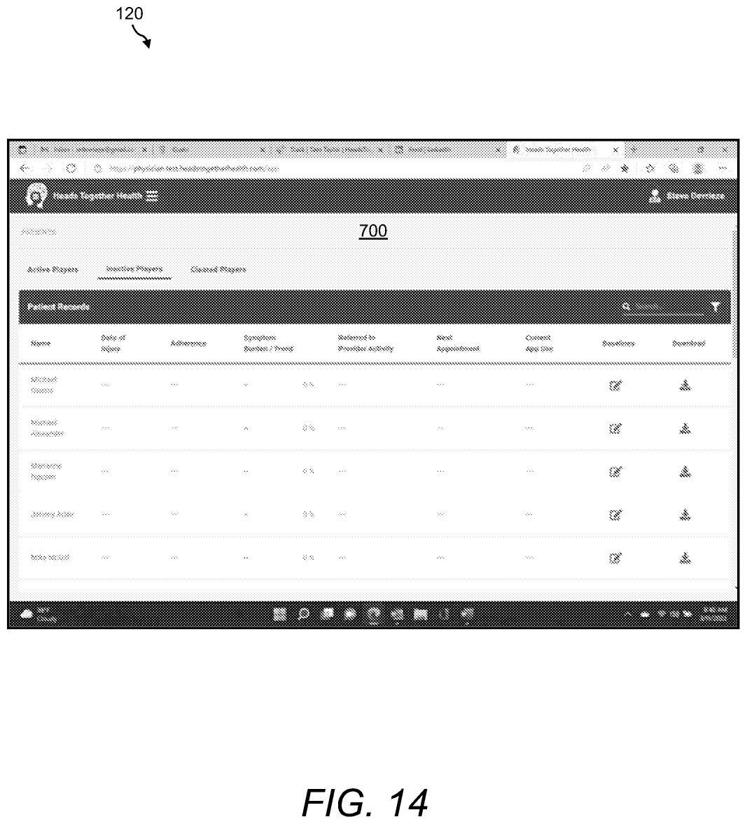

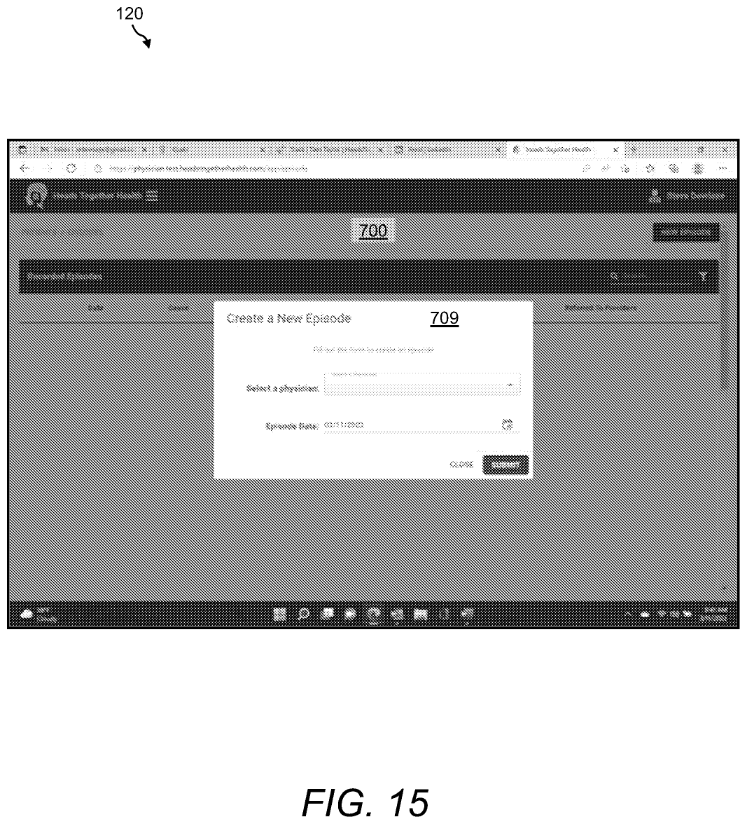

Referring now to FIG. 14, the healthcare provider 170 may move to a new patient, a new concussion episode, and review the data capture process. In this example, from the home page 700 of clinician web portal 120, the healthcare provider 170 may add a new Patient Concussion. For example, the healthcare provider 170 may select a patient from Inactive Players List to establish a new concussion patient episode (by clicking on their name). Next, the healthcare provider 170 may select a Physician and date from the drop-down list and then select “Submit,” as shown, for example, in FIG. 15. That is, FIG. 15 can show a New Episode menu 709.

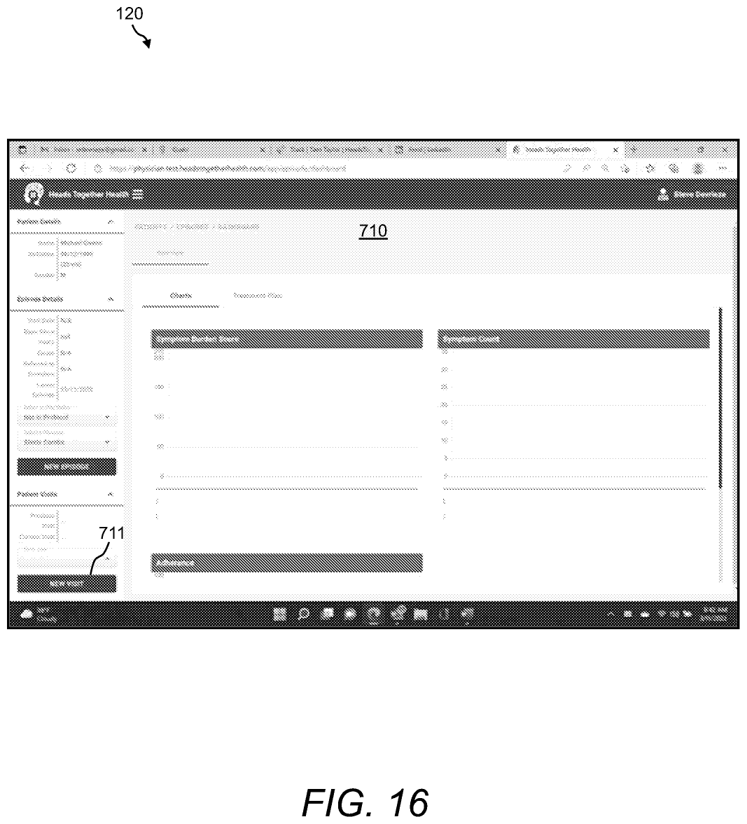

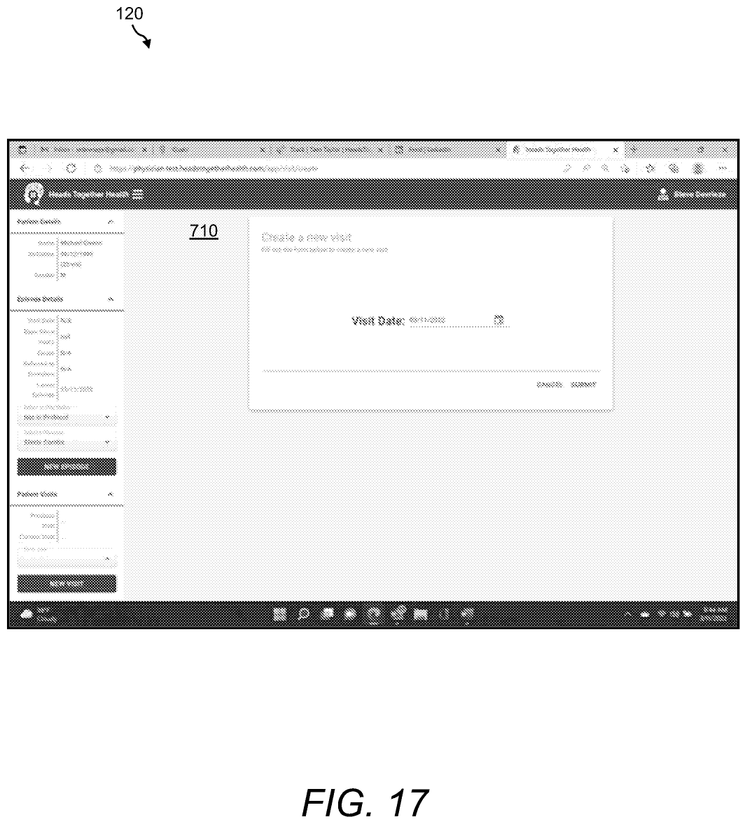

Next, FIG. 16 shows that a new episode dashboard screen 710 appears for the new patient and with no data. The healthcare provider 170 may create a new visit by selecting the “New Visit” button 711. Next, the healthcare provider 170 may select a visit date and select “Submit,” as shown, for example, in FIG. 17.

Referring now to FIG. 18, clinician web portal 120 may provide an Intake Form 712 with respect to Data Capture for New Visit. In this example, Intake Form 712 may contain three subcategories: Injury Information, Patient History and Symptom Qualifiers. Depending on the clinic workflow, the data capture in these three tabs may be gathered pre-physician visit. For example, the Injury information may be pre-gathered by an ATC who was present at time of the injury.

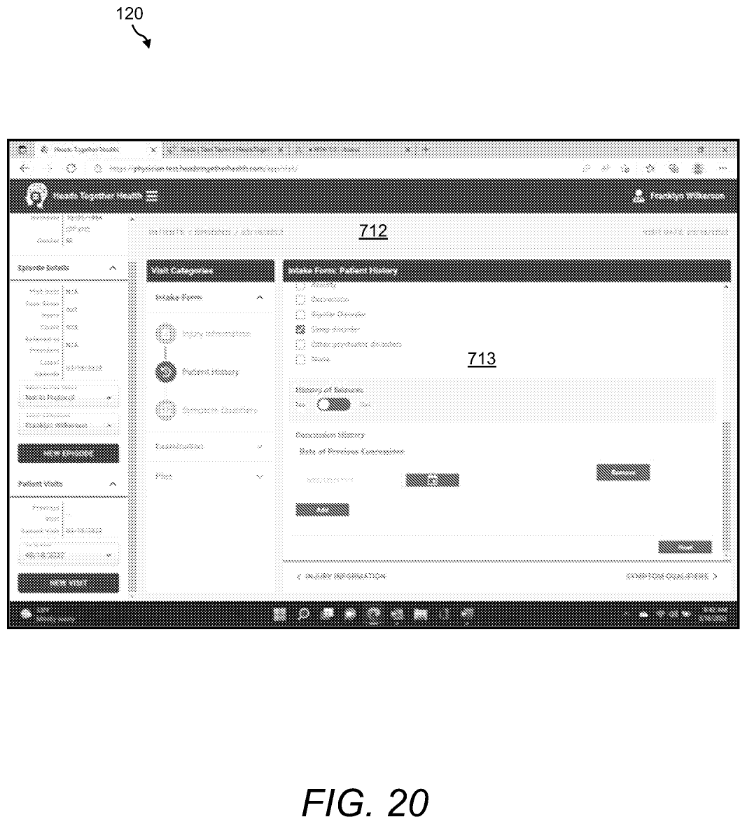

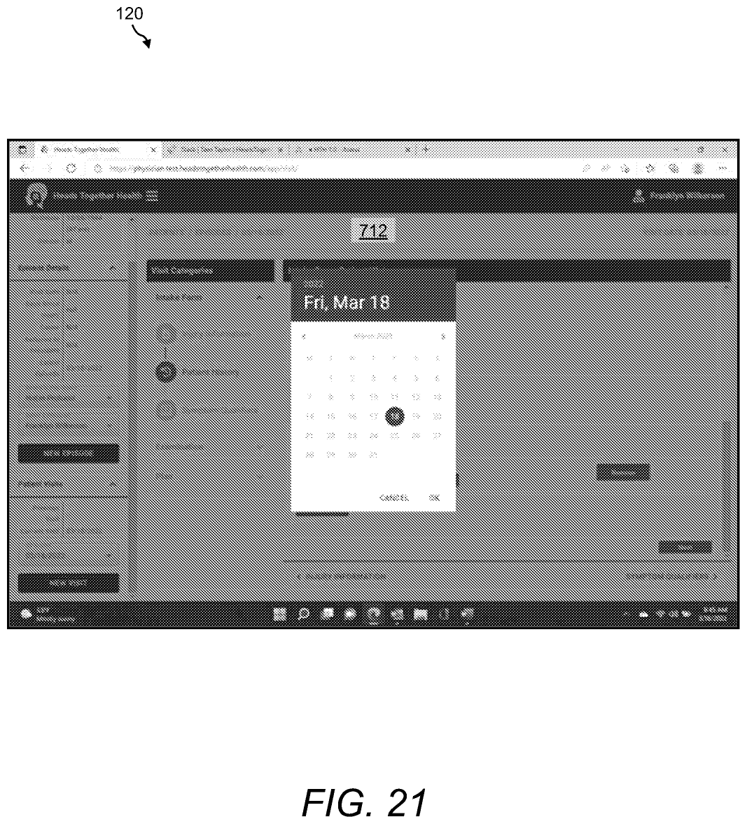

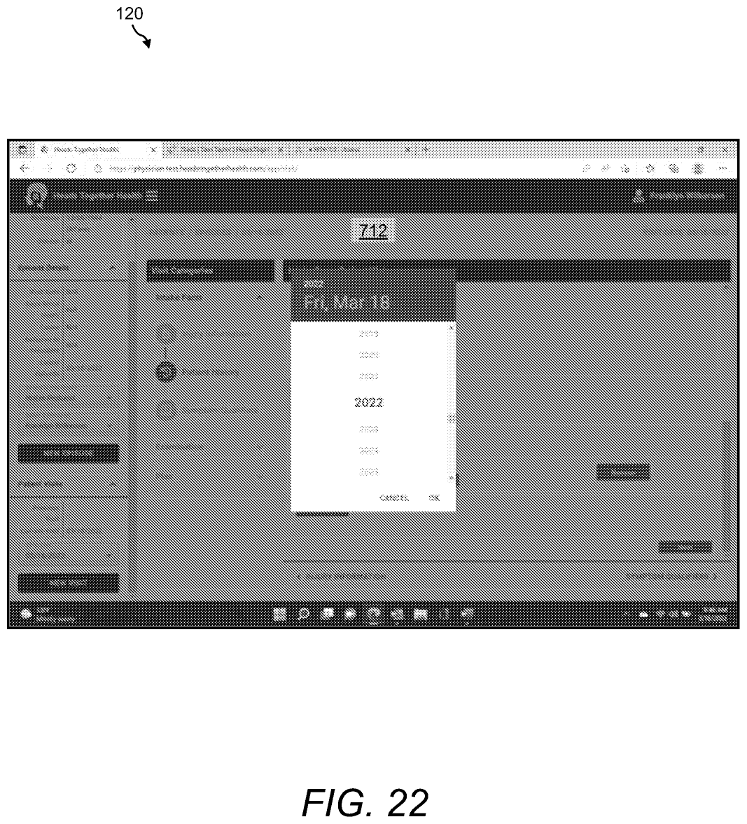

Using Intake Form 712, to advance to the next section, answering the questions and clicking the “Next” button can open the Patient History menu 713, as shown, for example, in FIG. 19. Next, at the Patient History menu 713 shown in FIG. 20, the healthcare provider 170 may answer the Patient History questions. Then, at the bottom of the Patient History tab, the healthcare provider 170 may enter previous concussions. Referring now to Patient History menu 713 shown in FIG. 21 and FIG. 22, when the healthcare provider 170 selects the date for a previous concussion, the year can be selected (e.g., as shown in FIG. 21 and FIG. 22).

Next, using Patient History menu 713, previous concussion data, family history, and social history may be populated. The “Next” and “Complete” buttons may be used as each screen is completed. In this example, the patient, concussion, and family history information is pre-dominantly related to gathering PCS risk data (e.g., gender, age, previous concussions, headaches, amnesia, ADHD, Psychological/Psychiatric and Sleep Disorders, etc.).

Next, and referring now to FIG. 23, upon completing of the Patient History section of Patient History menu 713, Symptom Qualifiers may be captured using a Symptom Qualifiers menu 714. In one example, this symptom capture may use a standard 0-6 rating scale. Additionally, this symptom capture may have some additional symptoms vs. the traditional SCAT5 22 symptom scale (particularly within vision). In this example, the healthcare provider 170 may select 0-6 for each symptom, and then answer the four questions at the bottom of the chart and then click “Next”.

Referring to Symptom Qualifiers menu 714 shown in FIG. 23, depending on symptoms selected, there may be follow-up questions which are meant to capture information that could be helpful insights into treatment needs. Note that these are optional per clinic workflow and preference because some clinics may not want to include these questions. Further, this subcategory may follow the same instructions as the previous: answer the questions and to advance to the next section click the “Next” button on the bottom right. Once all sections in Symptom Qualifiers are completed, select the “Completed” button.

Next, and referring to FIG. 24, the healthcare provider 170 may progress to the Examination portion of Intake Form 712. For example, a Physical Exam link 715 may be provided. This may be when the physician initiates engagement with the patient, and the results of the data intake could be reviewed by the physician before seeing the patient.

Having clicked on the Physical Exam link 715, a Physical Exam menu 716 may be displayed, as shown, for example, in FIG. 24. The Physical Exam subcategory may follow the same instructions as the previous: answer the questions and to advance to the next section within Physical Exam, click the “Next” button on the bottom right.

Once all sections in Physical Exam are completed, the “Complete” button may be selected. Then, the healthcare provider 170 may progress to the Tests portion of Intake Form 712. For example, an Additional Tests link 717 may be provided. Having clicked on the Additional Tests link 717, an Additional Tests menu 718 may be displayed, as shown, for example, in FIG. 25.

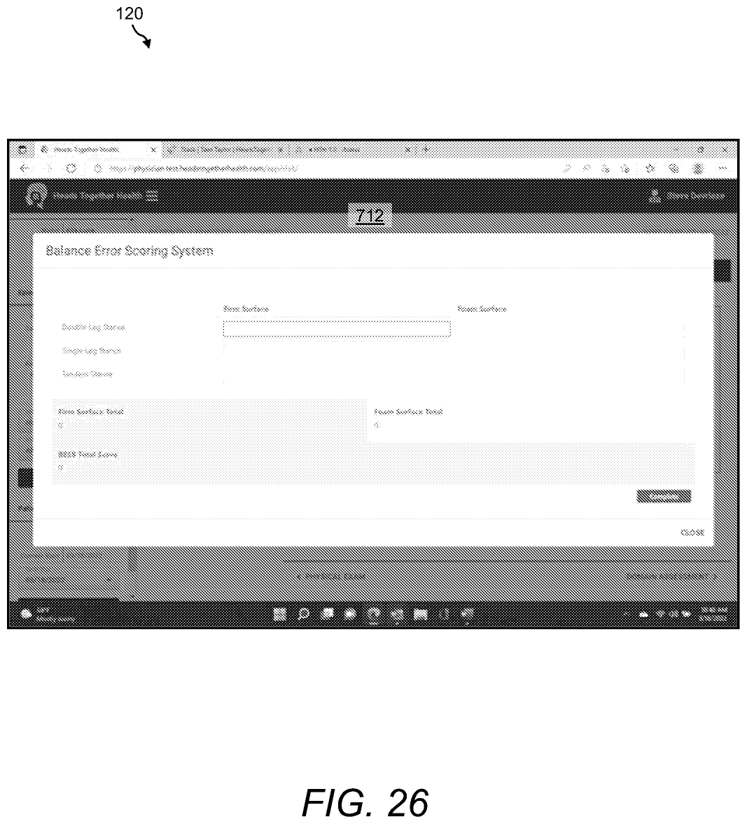

For example, within the Additional Tests menu 718, several optional tests may be provided. This section can vary by clinic and can allow for the physician to select which tests to execute based on the patient's situation. Further, this section allows for review of any available baseline tests (by clicking on “Baseline”), if they had been completed before. Further, using the Additional Tests menu 718, a new test may be created by selecting the radio button. Then, once the test option appears in the “Active Tests” box, the healthcare provider 170 may select “COMPLETE TEST”. The test data capture then appears, which allows the healthcare provider 170 may to capture the data. An example of a selection of a Balance Error Scoring Systems (BESS) test is shown in FIG. 26.

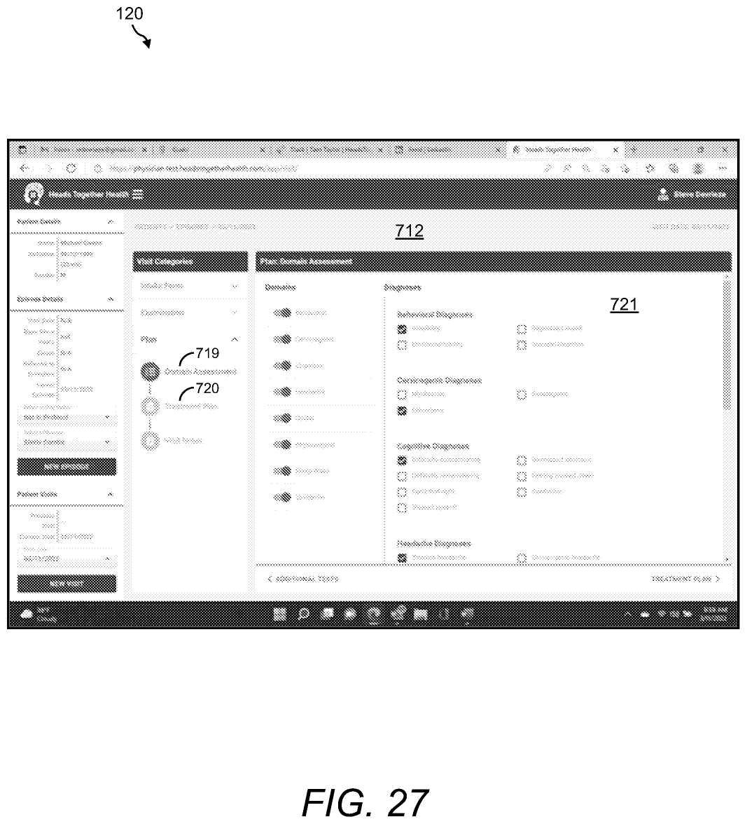

Next, and referring now to FIG. 27, upon completing any relevant tests, the healthcare provider 170 may proceed to a domain assessment 719-portion and/or a treatment plan 720-portion of clinician web portal 120. FIG. 27 also shows a domain assessment menu 721. At domain assessment 719, brain healthcare application 110 may leverage the results of the previous data (Symptom Qualifiers, Physical Exam, and Additional Tests) to identify what is impacting the patient. At domain assessment menu 721, the healthcare provider 170 may select a domain, which is then highlighted. Then, the diagnoses associated with the selected domain can be displayed. Then, to select a diagnosis, the healthcare provider 170 may select the box that matches with the diagnosis and a check will appear. Diagnoses may be customized by the clinic. Then, to unselect a domain, the healthcare provider 170 may select the highlighted domain and the domain is then unhighlighted.

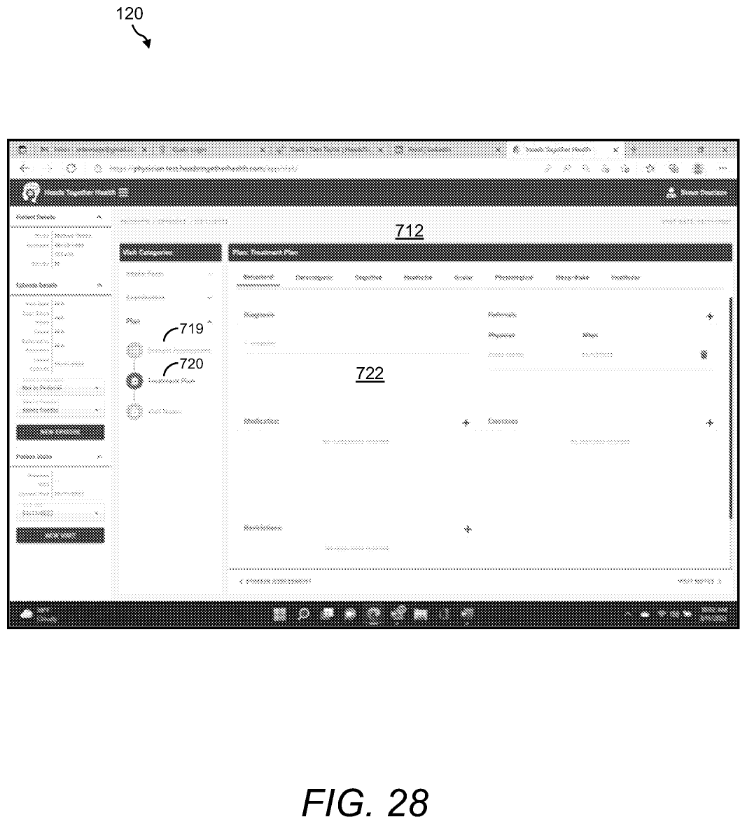

Next, and referring now to FIG. 28, upon establishing the affected domains, the healthcare provider 170 may derive a treatment plan by selecting treatment plan 720. FIG. 28 also shows a treatment plan menu 722. Treatment plan 720 and treatment plan menu 722 shown in FIG. 28 may show an example of a patient where all domains were selected as affected. Further, healthcare providers 170 may establish referral visits, medications, rehab exercise activities and restrictions for each diagnosis within each affected domain. This example only includes functionality for the physician to establish treatment plan inputs. However, in other embodiments, methodology may be employed to leverage data to provide insights for the treatment plan based on previous data capture. Further, using this methodology, optimal recoveries for patients with similar data attributes (our phenotyping methodology) can be identified. Further, the selections across these Treatment Plan options may be customizable by the using clinic.

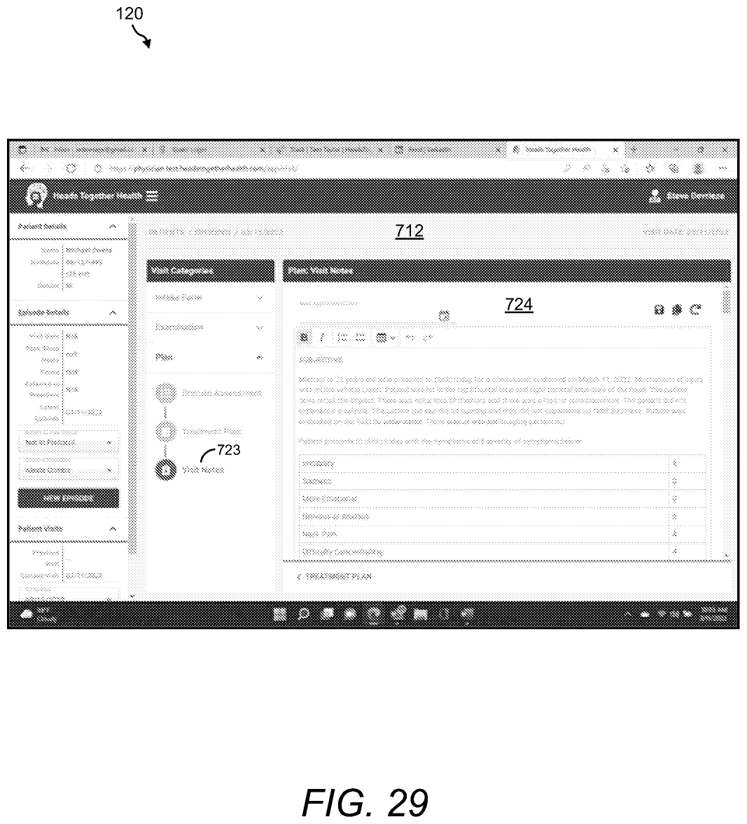

Next, and referring to FIG. 29, once all of the relevant treatment selections have been made, the healthcare provider 170 may proceed to a visit notes 723-portion of clinician web portal 120. The visit notes 723-portion can have a visit notes menu 724. Using the visit notes menu 724, the next appointment date may be set and an overview of the patient's visit and the data capture information is automatically generated. Here, healthcare providers 170 may make text adjustments to several sections of the note. Here, healthcare providers 170 may copy/paste the note to an EMR Notes tab (in HTML format). Then, to complete the visit, healthcare providers 170 may select a “Complete Visit” button.

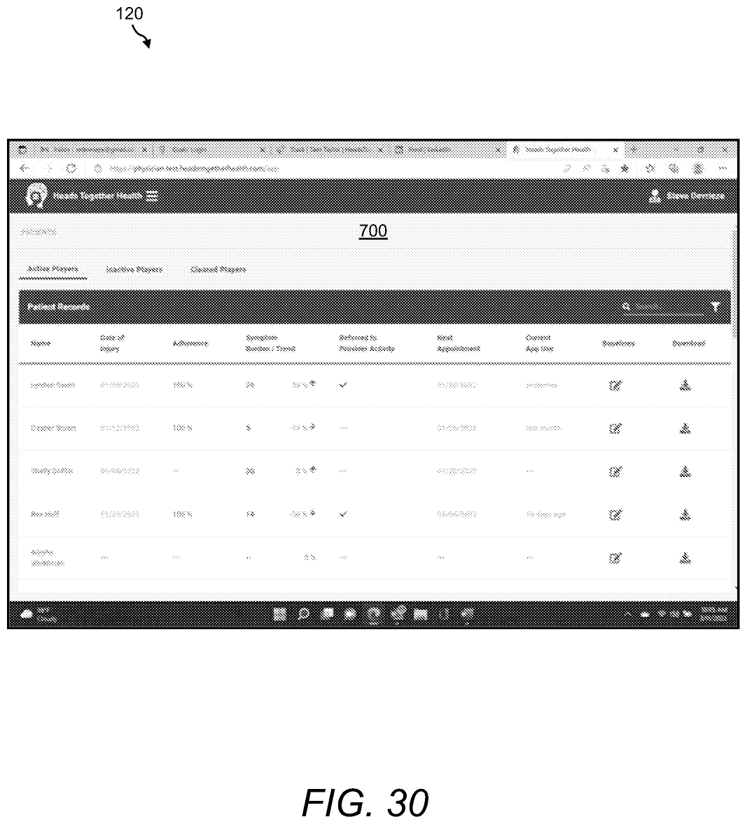

Next, and referring now to FIG. 30, following completion of the visit, healthcare providers 170 may will be directed back to the Active Players page of home page 700 (and the patient for whom an Episode was created and now appearing in the “Active” category).

FIG. 31 through FIG. 52 show an example of a process of using brain health mobile app 162 of brain healthcare application 110 of healthcare system 100. Brain health mobile app 162 may be a custom interface that may be used by patients 105. For example, brain health mobile app 162 may be designed for both collecting symptom and treatment activity adherence data from patients 105, and providing information back to patients 105. Patients 105 may be automatically prompted by brain health mobile app 162 to account for changes in activities or PRMs. Automatic prompts can allow important clinically relevant information to be appended to the incoming data streams. Patients 105 may also submit feedback from outside the clinic, at any time of day or night. More frequent and consistent approach to the subjective data can allow more information to be gathered than having medical professionals (e.g., doctors and nurses) collecting data only during office visits.

In some cases, data from the patients can be obtained autonomously. Wearable devices can be used by the patients to collect data that can be used to provide useful information to medical professionals and/or for training a machine learning algorithm. The wearable device can be, for example, a bracelet, a computer, a mobile device, an epidermal, an earbud, a headphone, a fabric, a shoe, an implant, glasses, a tracker, a belt, a sock, a shirt, or a ring. The wearable device may be in operable communication with a mobile device of the patient, a server, and a computer of a medical professional to transmit information.

In some aspects, the present disclosure provides a computer-implemented method for optimizing a treatment for a traumatic brain injury in a subject. The method can comprise receiving activity data of the subject, wherein the subject has received a treatment for the traumatic brain injury. The activity data can relate to whether the subject has adhered to the treatment. The method can comprise applying a machine learning model to the activity data to predict whether the subject should continue the treatment or switch to a different treatment.

In some cases, the machine learning model can predict that the subject should switch to the different treatment. The different treatment can comprise a duration or a frequency that is different than a previous duration or a previous frequency of a previous treatment delivered to the subject for the traumatic brain injury. For instance, if activity data indicates that the subject's condition has improved significantly, the duration or the frequency of a new treatment can be lower than the previous treatment. Conversely, if activity data indicates that the subject's condition has worsened or not improved as much as expected, the duration or the frequency of a new treatment can be lower than the previous treatment.

FIG. 31 shows, for example, the functionality of brain health mobile app 162. For example, using brain health mobile app 162, patients 105 may self-report symptoms, the severity of their symptoms, activities which most impact symptoms, and the timing of their symptoms. This may be a TRACK SYMPTOMS selection. This information is accessible to healthcare providers 170 via clinician web portal 120.

Further, using brain health mobile app 162, patients 105 may track their treatment plan and their adherence to the activities prescribed by the healthcare provider 170. This information can be accessible to healthcare providers 170 via clinician web portal 120. This may be a REHAB PLAN selection.

Further, brain health mobile app 162 may provide educational materials customized for concussion management, including diagnosis summary, treatment plan details, explanations and instructions for designated exercises and upcoming appointments. This may be a LEARNING CENTER selection. For example, PDFs or videos may be available for any exercise rehab activities where instructions would benefit the experience.

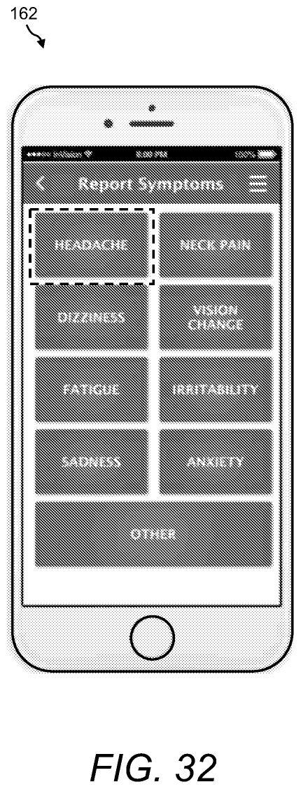

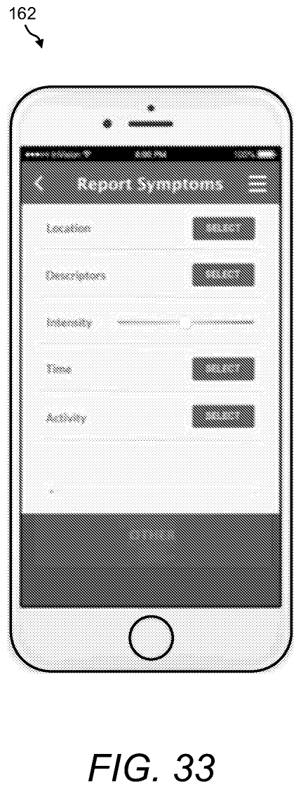

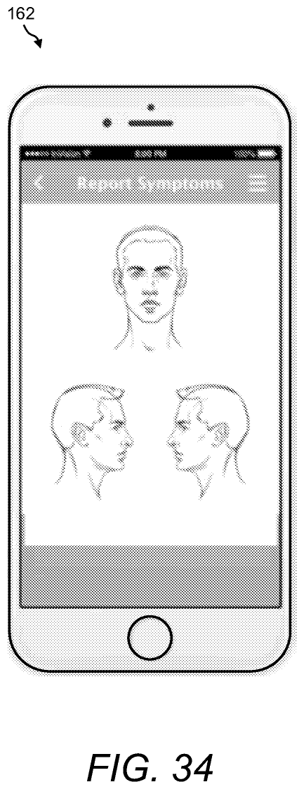

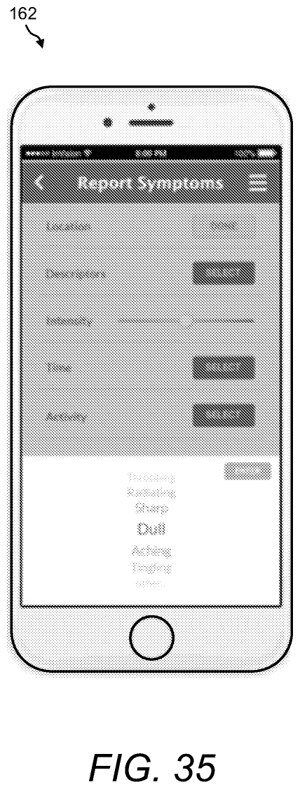

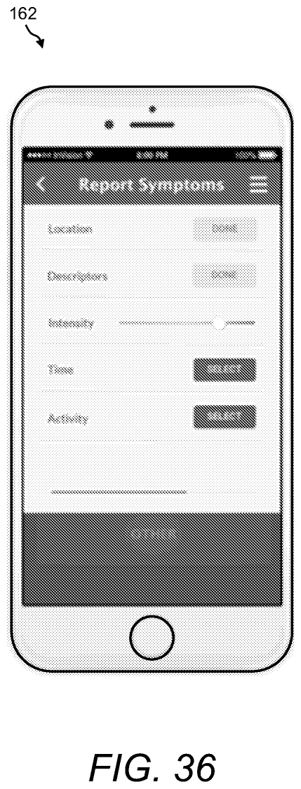

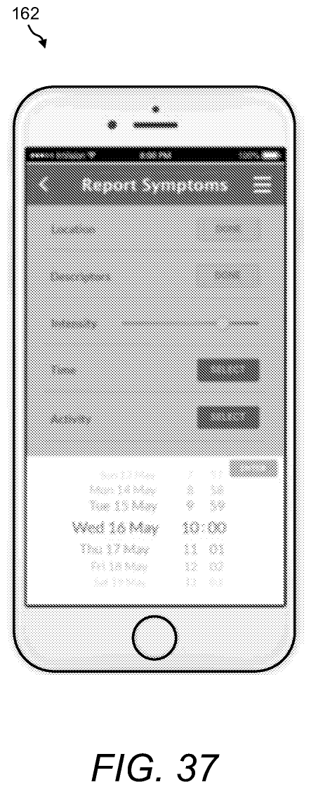

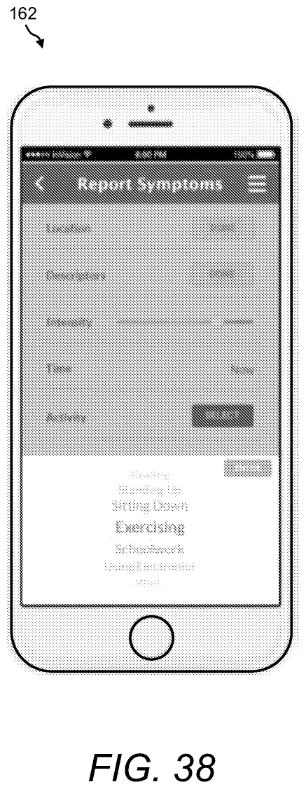

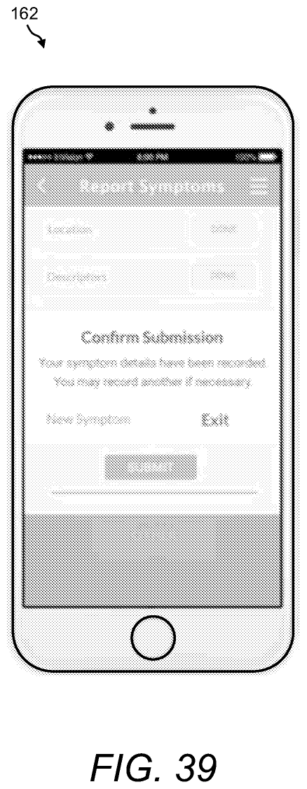

Referring now to FIG. 32 through FIG. 39, brain health mobile app 162 may be used with respect to the TRACK SYMPTOMS selection. That is, brain health mobile app 162 may be used by patients 105 for symptom reporting, as shown, for example, in FIG. 32 through FIG. 39. For example, patients 105 may self-report symptoms for those which are relevant (e.g., headache). Further to the example, brain health mobile app 162 may be used by patients 105 for individual symptom reporting, as shown, for example, in FIG. 33 through FIG. 39. For example, patients 105 may self-report symptom burden. In this example, headache (see FIG. 32) is the symptom being reported. Here, the patient 105 may report certain things about the headache (see FIG. 33), such as, but not limited to, location (see FIG. 34), pain description (see FIG. 35), intensity (see FIG. 36), time of day (see FIG. 37), and activities which cause headache (see FIG. 38). Upon entering this information, patients 105 may submit the information to brain healthcare application 110 (see FIG. 39).

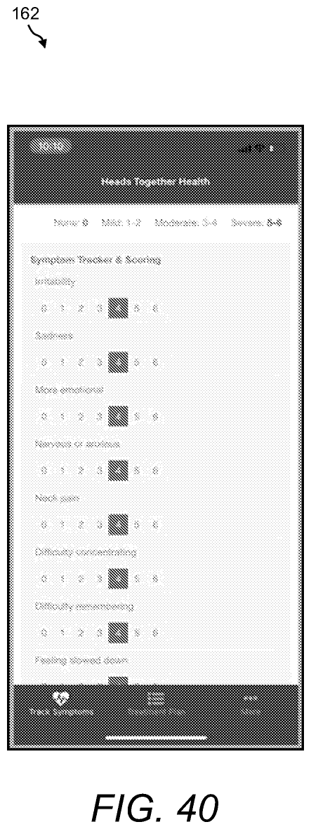

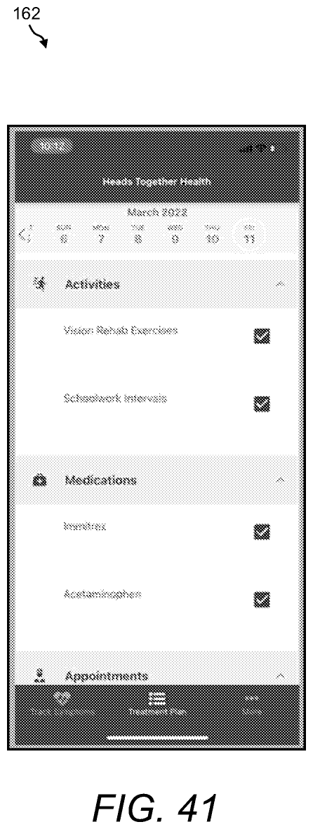

Referring now to FIG. 40 and FIG. 41, patients 105 may use brain health mobile app 162 to track symptom burden via a standard 0-6 rating scale (see FIG. 40), and by answering four standard questions (see FIG. 41). Further, on the “Treatment Plan” tab at the bottom (see FIG. 41), patients 105 may review their treatment plan activities, medications, referrals, and restrictions, and record daily progress against these items.

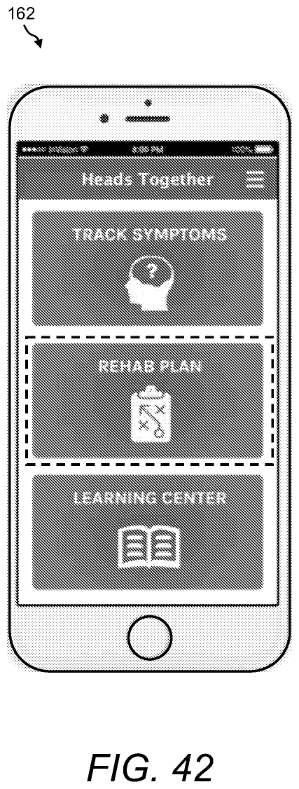

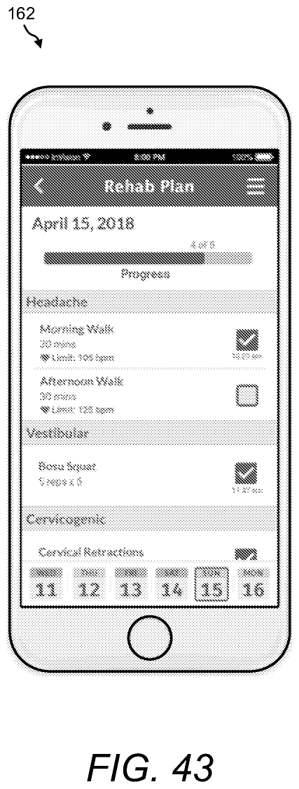

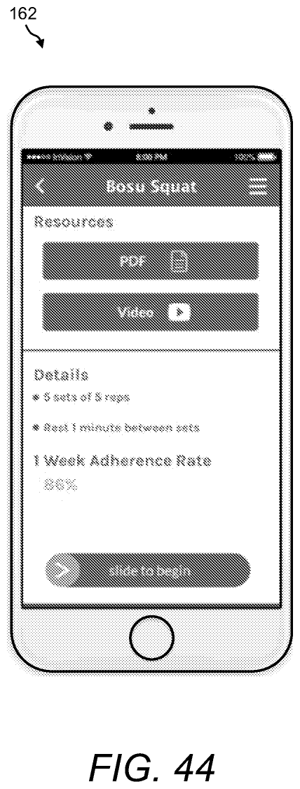

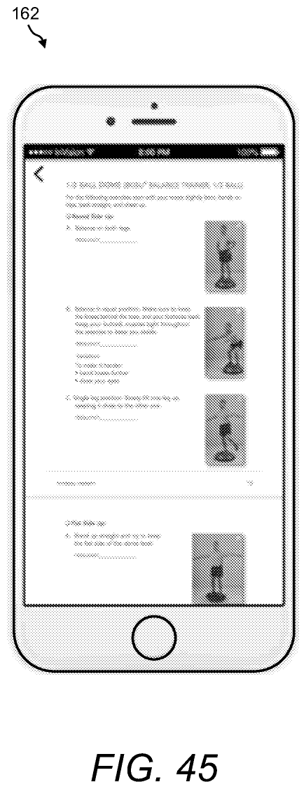

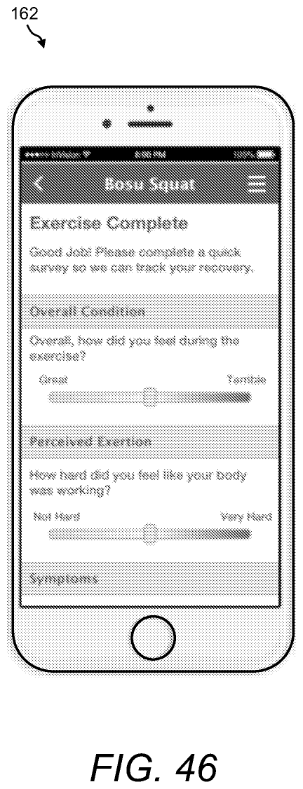

Referring now to FIG. 42 through FIG. 46, brain health mobile app 162 may be used with respect to the REHAB PLAN selection. For example, the REHAB PLAN selection may provide patients 105 a guide to their rehab plan. For example, patients 105 may review their rehab plan, self-report adherence to the plan, and review activity instructions (see FIG. 43). Further, patients 105 may review activity or exercise instructions (see FIG. 44 and FIG. 45) and report their condition and exertion following completion of the exercises (see FIG. 46).

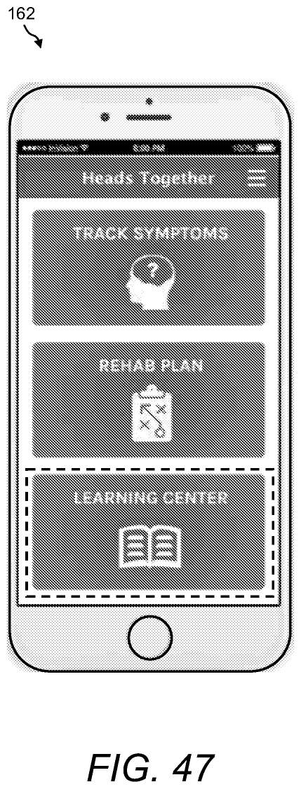

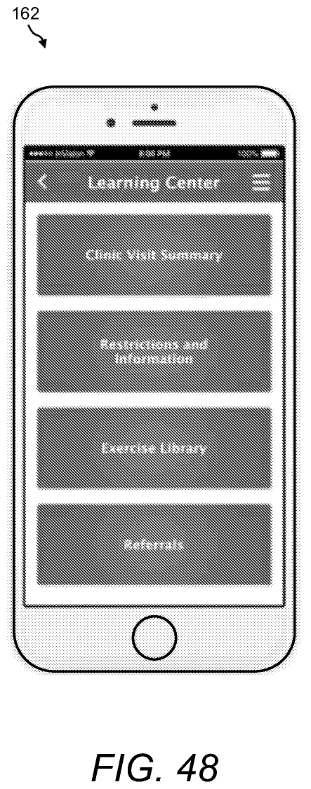

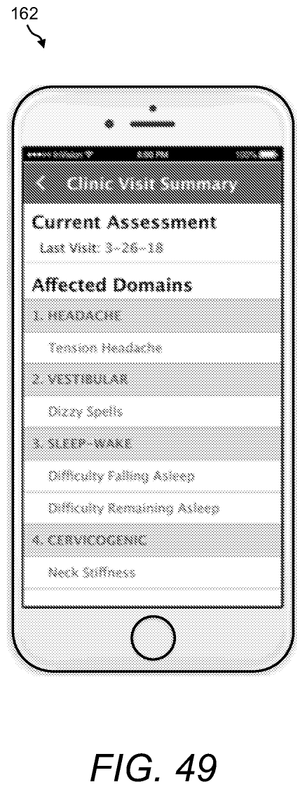

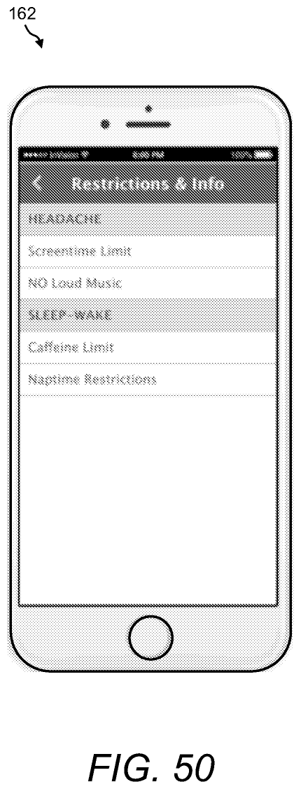

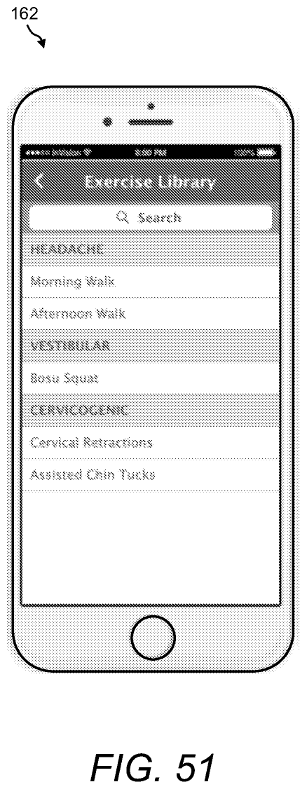

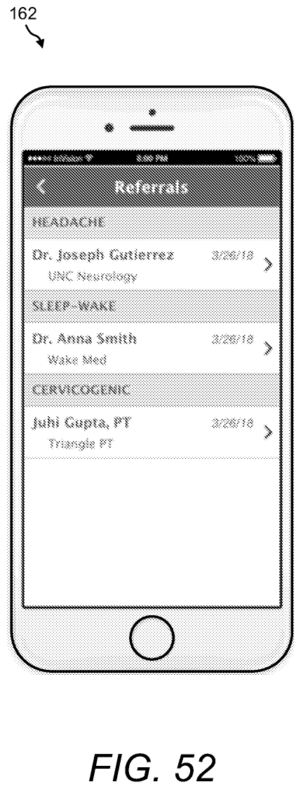

Referring now to FIG. 47 through FIG. 52, brain health mobile app 162 may be used with respect to the LEARNING CENTER selection. For example, FIG. 48 shows that patients 105 may access a summary of their visit, any activity restrictions, access to a full exercise library, and access to upcoming referrals (if desired and integrated to EPIC). FIG. 49 shows an example of a clinical visit summary. FIG. 50 shows an example of restrictions and information. FIG. 51 shows an example of an exercise library. FIG. 52 shows an example of upcoming referrals.

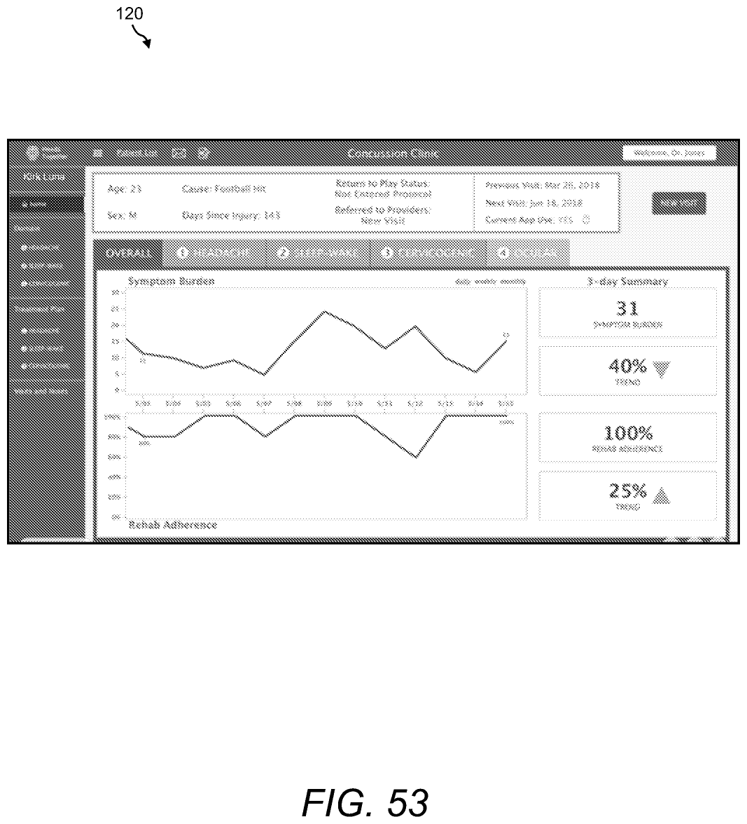

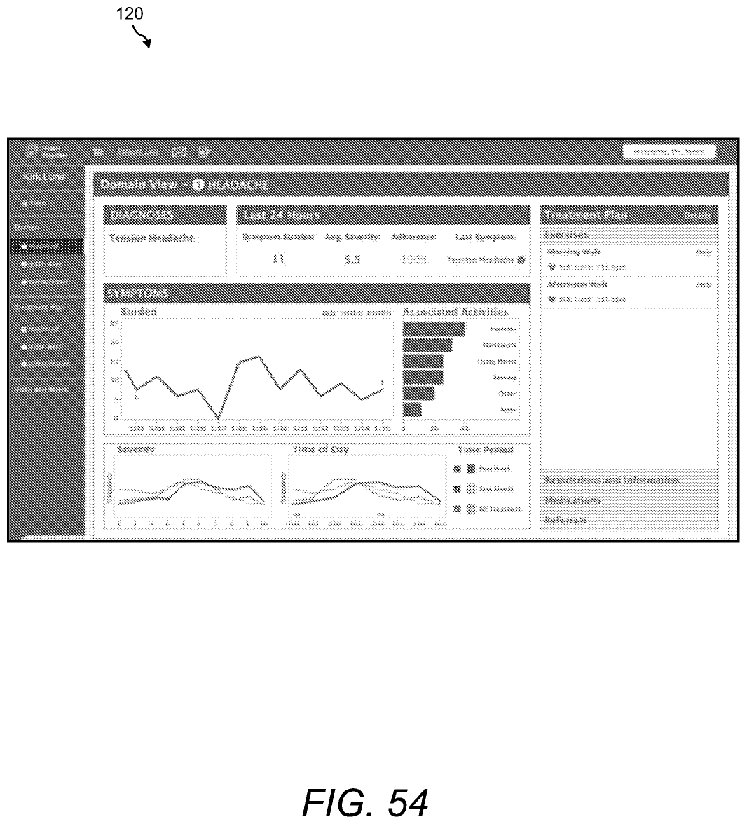

FIG. 53 and FIG. 54 now return back to clinician web portal 120 (or patient dashboard 120) of brain healthcare application 110 of healthcare system 100. Here, following data entry by the patient 105 in brain health mobile app 162, the healthcare provider 170 may login to clinician web portal 120. Accordingly, healthcare provider 170 may see the symptom tracking and treatment adherence updates that have been made by the patient 105 (depicting the data that the healthcare provider 170 can see in between visits). For example, FIG. 53 shows a screenshot of clinician web portal 120 depicting individual patient summaries. FIG. 54 shows a screenshot of clinician web portal 120 depicting clinical domain-specific tracking.

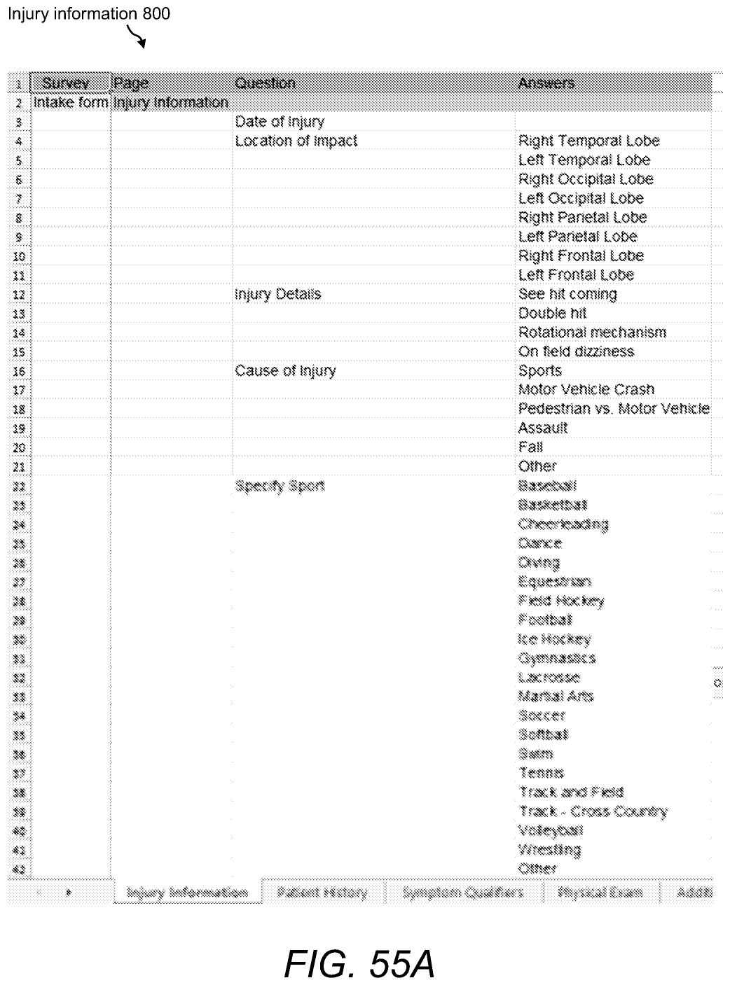

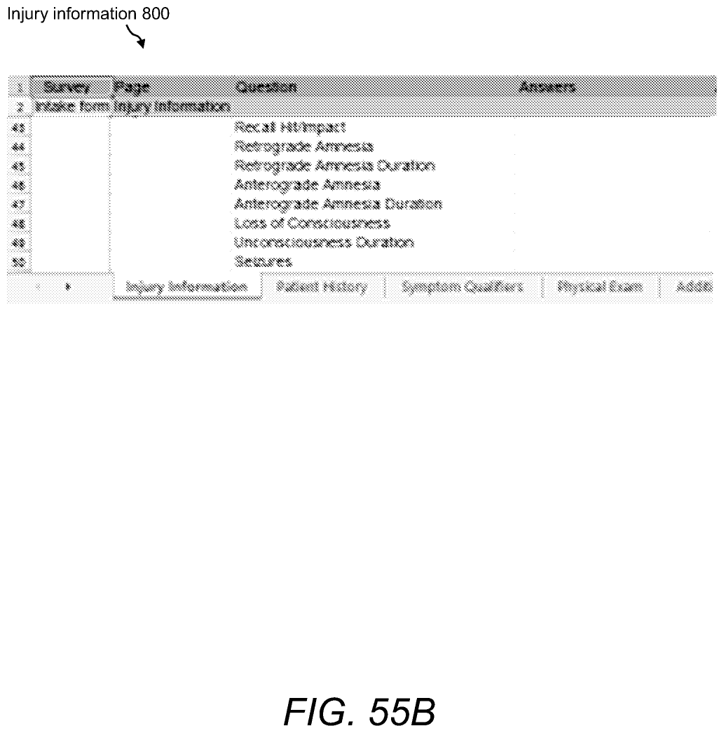

FIG. 55A through FIG. 61 show screenshots of an example of the information structure supporting clinician web portal 120 and/or brain health mobile app 162 of the healthcare system 100 for managing brain injury or concussion. In this example, the information structure is presented in spreadsheet form.

In one example, FIG. 55A and FIG. 55B show injury information 800. Injury information 800 depicts an example of the information structure supporting, for example, Intake Form 712 of clinician web portal 120 (see FIG. 17 through FIG. 30). Injury information 800 can include, for example, Questions and Answers that may be built into Intake Form 712 of clinician web portal 120. The injury information 800 shown in FIG. 55A and FIG. 55B may be just a portion of the injury information needed to fully support Intake Form 712. By way of example, FIG. 18 shows an example of Intake Form 712 for processing injury information 800.

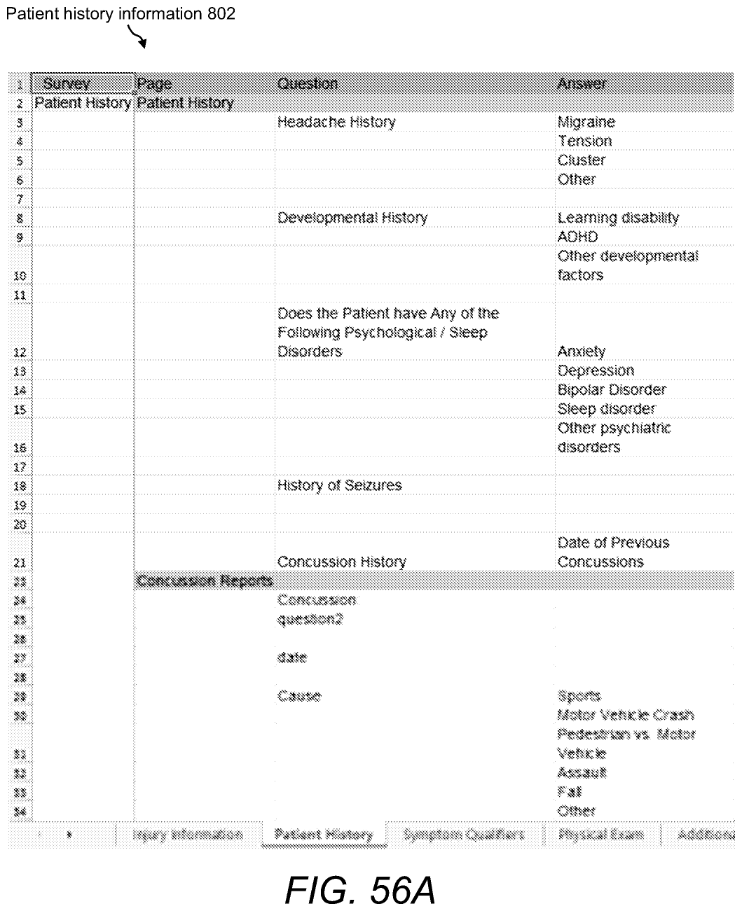

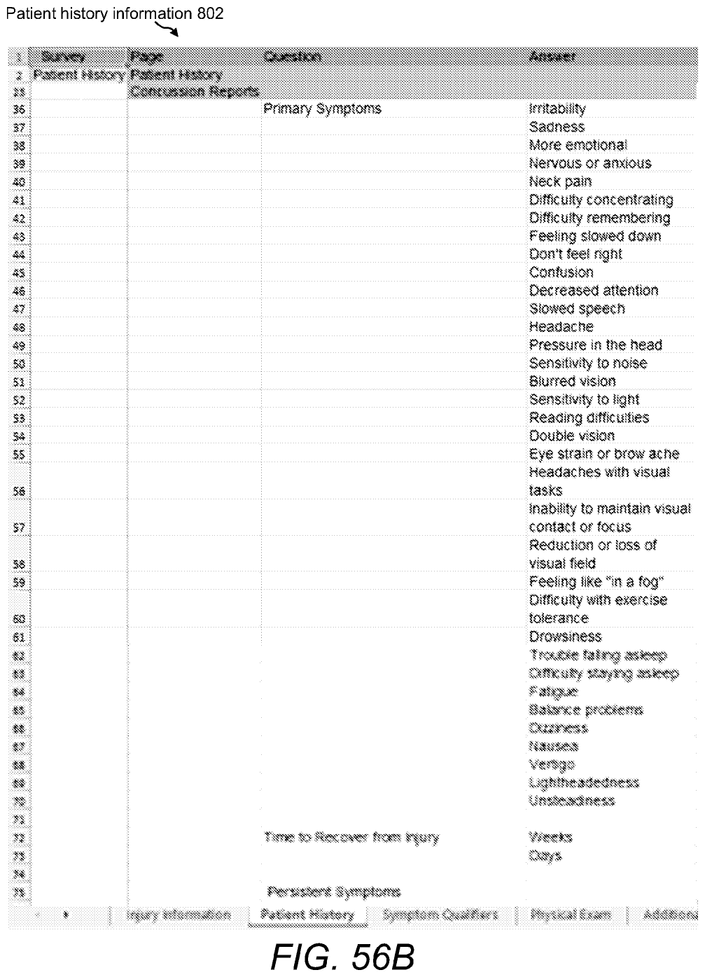

In another example, FIG. 56A and FIG. 56B show patient history information 802. Patient history information 802 depicts an example of the information structure supporting, for example, Intake Form 712 of clinician web portal 120 (see FIG. 17 through FIG. 30). Patient history information 802 can include, for example, Questions and Answers that may be built into Intake Form 712 of clinician web portal 120. The patient history information 802 shown in FIG. 56A and FIG. 56B may be just a portion of the patient history information needed to fully support Intake Form 712. By way of example, FIG. 19 through FIG. 22 shows an example of Intake Form 712 for processing patient history information 802.

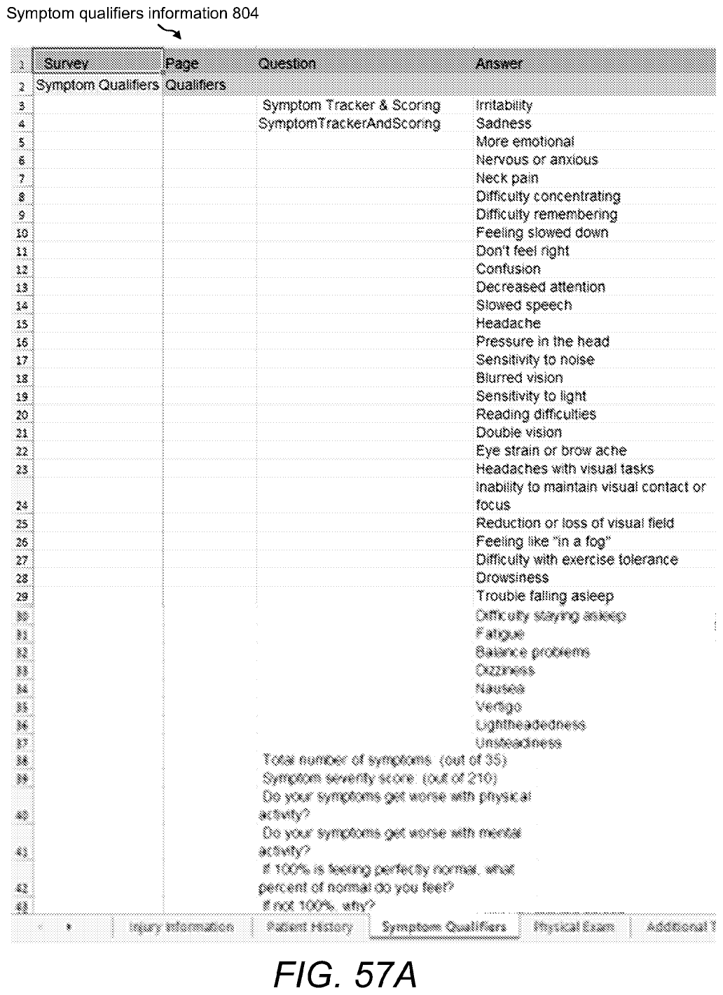

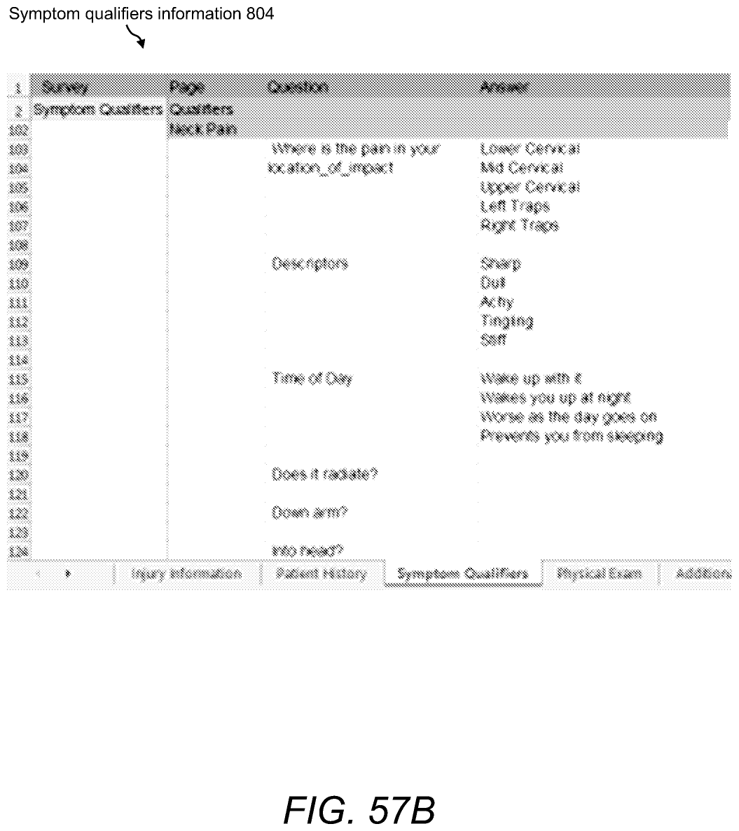

In yet another example, FIG. 57A and FIG. 57B show symptom qualifiers information 804. Symptom qualifiers information 804 depicts an example of the information structure supporting, for example, Intake Form 712 of clinician web portal 120 (see FIG. 17 through FIG. 30).

Symptom qualifiers information 804 can include, for example, Questions and Answers that may be built into Intake Form 712 of clinician web portal 120. The symptom qualifiers information 804 shown in FIG. 57A and FIG. 57B may be just a portion of the symptom qualifiers information needed to fully support Intake Form 712. By way of example, FIG. 23 shows an example of Intake Form 712 for processing symptom qualifiers information 804.

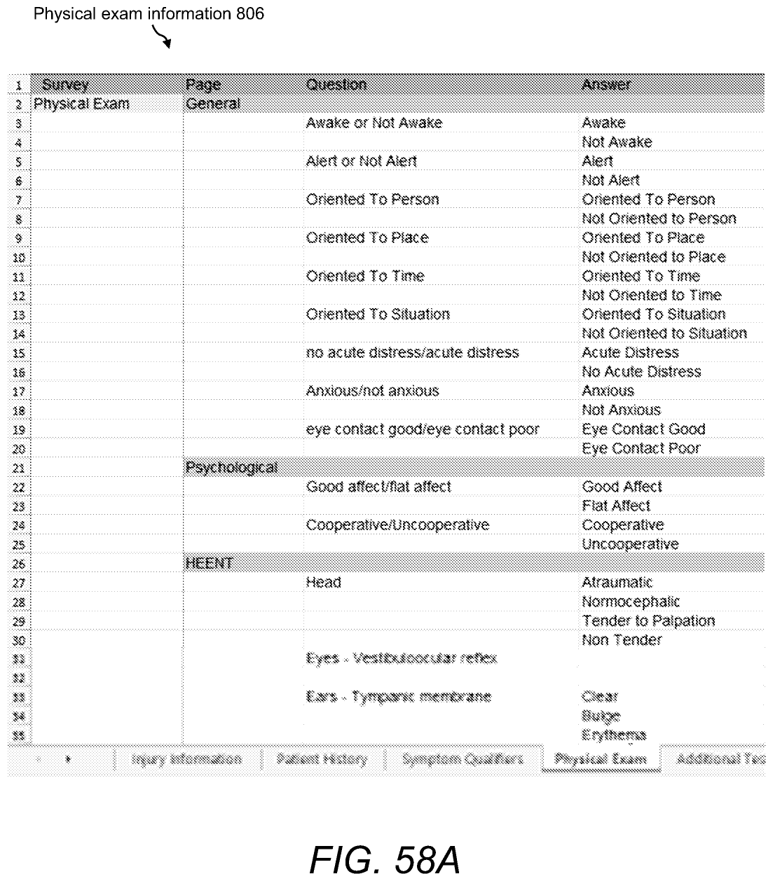

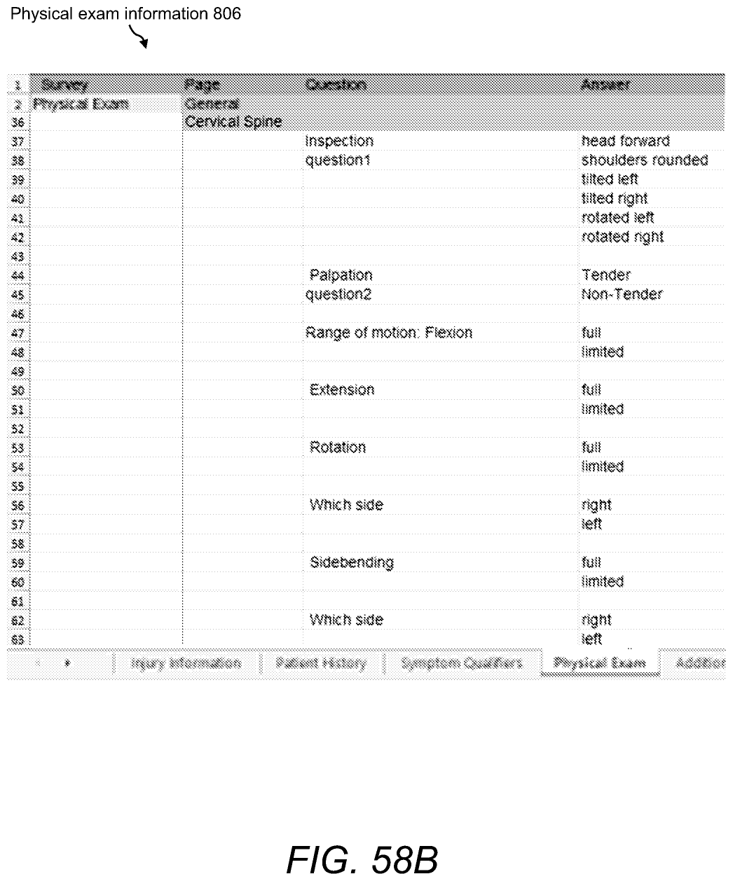

In yet another example, FIG. 58A and FIG. 58B show physical exam information 806. Physical exam information 806 depicts an example of the information structure supporting, for example, Intake Form 712 of clinician web portal 120 (see FIG. 17 through FIG. 30). Physical exam information 806 can include, for example, Questions and Answers that may be built into Intake Form 712 of clinician web portal 120. The physical exam information 806 shown in FIG. 58A and FIG. 58B may be just a portion of the physical exam information needed to fully support Intake Form 712. By way of example, FIG. 24 shows an example of Intake Form 712 for processing physical exam information 806.

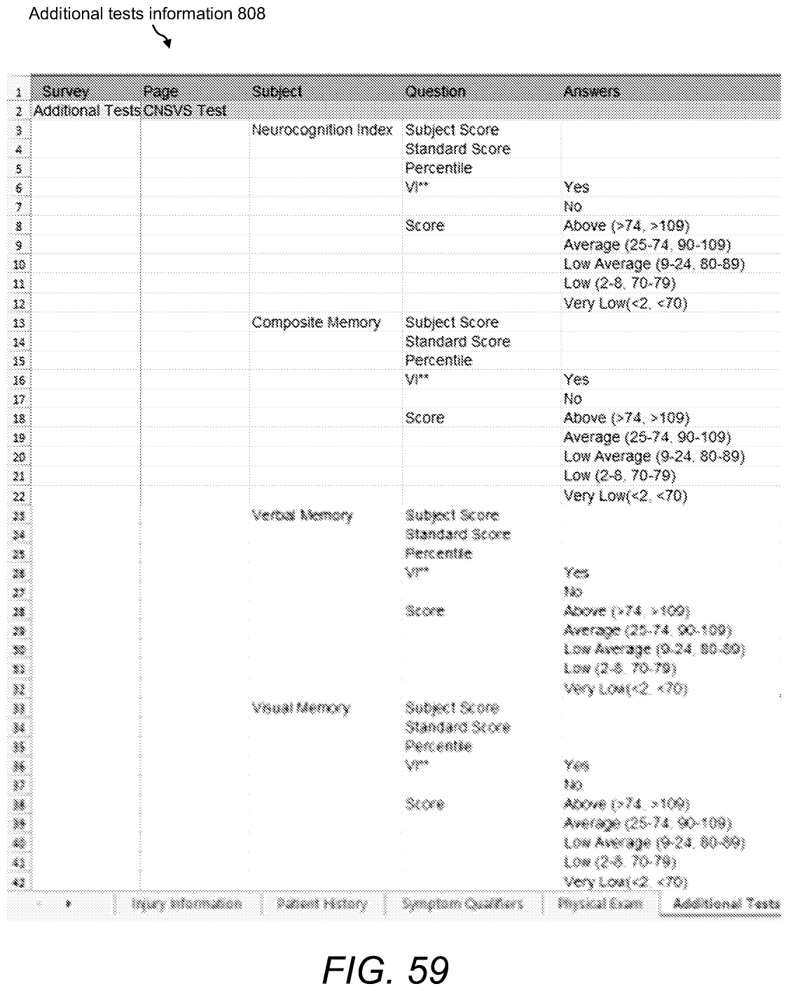

In yet another example, FIG. 59 shows additional tests information 808. Additional tests information 808 depicts an example of the information structure supporting, for example, Intake Form 712 of clinician web portal 120 (see FIG. 17 through FIG. 30). Additional tests information 808 can include, for example, Questions and Answers that may be built into Intake Form 712 of clinician web portal 120. The additional tests information 808 shown in FIG. 59 may be just a portion of the additional tests information needed to fully support Intake Form 712. By way of example, FIG. 25 shows an example of Intake Form 712 for processing additional tests information 808.

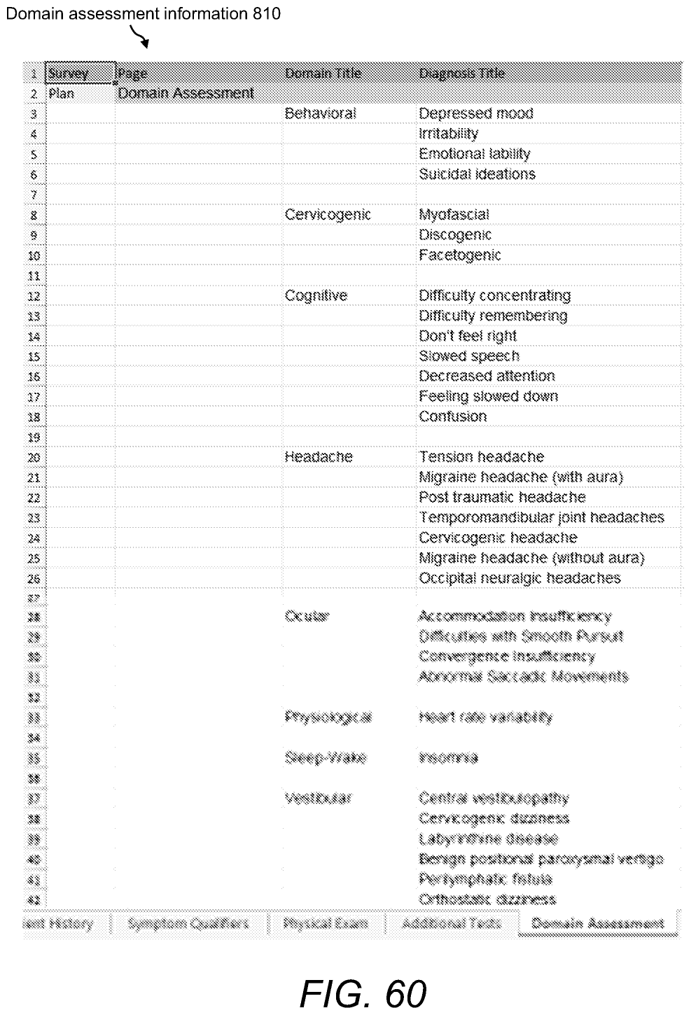

In yet another example, FIG. 60 shows domain assessment information 810. Domain assessment information 810 depicts an example of the information structure supporting, for example, Intake Form 712 of clinician web portal 120 (see FIG. 17 through FIG. 30). Domain assessment information 810 may include, for example, diagnoses with respect to the Behavioral, Cervicogenic, Cognitive, Headache, Ocular, Physiologic, Sleep-Wake, and Vestibular domains. The domain assessment information 810 shown in FIG. 60 may be just a portion of the domain assessment information needed to fully support Intake Form 712. By way of example, FIG. 27 shows an example of Intake Form 712 for processing domain assessment information 810.

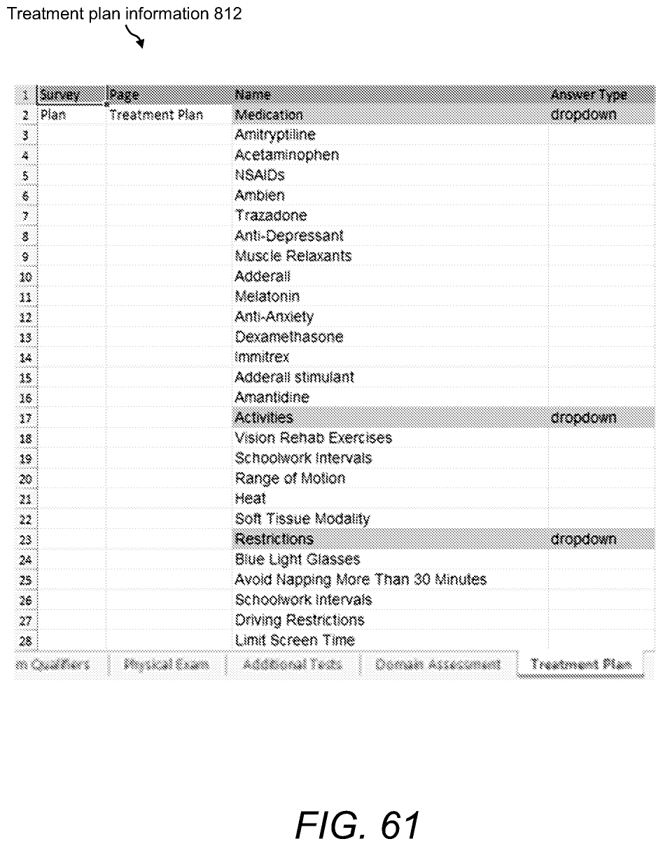

In still another example, FIG. 61 shows treatment plan information 812. Treatment plan information 812 depicts an example of the information structure supporting, for example, Intake Form 712 of clinician web portal 120 (see FIG. 17 through FIG. 30). Treatment plan information 812 may include, for example, a selection of Medications, Activities, and Restrictions. The treatment plan information 812 shown in FIG. 61 may be just a portion of the treatment plan information needed to fully support Intake Form 712. By way of example, FIG. 28 shows an example of Intake Form 712 for processing treatment plan information 812.

Referring to FIG. 1 through FIG. 61, the healthcare system 100 and methods (e.g., workflow 200, concussion subtyping process 250, system data flow and architecture 300, clinician web portal flow 400, clinician workflow 500, and mobile app process flow 600) may be provided for managing mTBI or concussion.

Further, the healthcare system 100 and methods (e.g., 200, 250, 300, 400, 500, 600) may provide a digital health platform that may capture and leverage clinically customized structured data to enable machine learning (ML) treatment insights from real-world concussion patient data.

Further, the healthcare system 100 and methods (e.g., 200, 250, 300, 400, 500, 600) may provide a digital health platform including machine learning models that may be trained to generate phenotypes based on patient and injury characteristics and symptoms across certain clinical domains, such as, but not limited to, behavioral, cervicogenic, cognitive, headache, ocular, physiologic, sleep-wake, and vestibular.

Further, the healthcare system 100 and methods (e.g., 200, 250, 300, 400, 500, 600) may provide brain healthcare application 110 running on application server 150 and accessible via network 155.

Further, the healthcare system 100 and methods (e.g., 200, 250, 300, 400, 500, 600) may provide brain healthcare application 110 including multiple algorithms, such as, but not limited to, ML algorithm 114, PCS prediction algorithm 116, and clustering algorithm 118.

Further, the healthcare system 100 and methods (e.g., 200, 250, 300, 400, 500, 600) may provide brain healthcare application 110 including robust analytics utilizing world class machine learning to inform an optimal treatment plan for similar patient segments and similar concussion or brain injury types. Currently, there is an inability for healthcare providers to learn from the best outcomes available in real-time, which leads to care inequality.

Further, the healthcare system 100 and methods (e.g., 200, 250, 300, 400, 500, 600) may provide brain healthcare application 110 including clinician web portal 120 and brain health mobile app 162, which may be custom interfaces with respect to treating mTBI or concussion.

Further, the healthcare system 100 and methods (e.g., 200, 250, 300, 400, 500, 600) may provide clinician web portal 120 including a structured data capture intake form to be used at diagnosis and during the patient treatment process.

Further, the healthcare system 100 and methods (e.g., 200, 250, 300, 400, 500, 600) may provide clinician web portal 120 featuring clinically customized concussion data capture for the purposes of enabling:
(1) machine learning algorithms to phenotype concussions based on clustering numerous data attributes (e.g., patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, physical exam results, wearable physiologic data, imaging data, history of previous recovery trajectories and treatments, heart rate variability, physical activity data, blood pressure, perspiration, calories burned, steps walked, body temperature, injury characteristics, symptoms, previous treatments, previous recovery timelines, or any combination thereof);
(2) machine learning algorithms to generate treatment insights for the clinician (i.e., based on the phenotype) that can support the care plan; and
(3) integrated technology solution which links data between the clinician web portal, EMR, and a patient mobile app, which enables patient reported recovery data.

Further, the healthcare system 100 and methods (e.g., 200, 250, 300, 400, 500, 600) may provide a communication platform for the patient, caregiver, and/or healthcare multidisciplinary team, including the customized clinician web portal 120 and/or the customized brain health mobile app 162.

Further, the healthcare system 100 and methods (e.g., 200, 250, 300, 400, 500, 600) may provide brain healthcare application 110 that features (1) structured data intake, (2) data aggregation, (3) machine learning model, and (4) dashboard reports and insights.

Further, the healthcare system 100 and methods (e.g., 200, 250, 300, 400, 500, 600) may provide a brain healthcare platform for delivering personalized treatment, including real-time feedback and data collection.

Further, the healthcare system 100 and methods (e.g., 200, 250, 300, 400, 500, 600) may provide a brain healthcare platform for providing customized treatment insights for improved management of concussion symptoms compared with the SOC alone.

Further, the healthcare system 100 and methods (e.g., 200, 250, 300, 400, 500, 600) may be provided for managing multiple other healthcare needs, such as, but not limited to, sports injuries, COVID risk assessment, back pain, musculoskeletal conditions and stroke, among others.

The systems and the methods of the present disclosure may be implemented using hardware, software, or a combination thereof and may be implemented in one or more computer systems or other processing systems. In one aspect, the present disclosure provides one or more computer systems capable of carrying out one or more functionalities described herein.

Various modifications and variations of the disclosed methods, compositions and uses of the invention will be apparent to the skilled person without departing from the scope and spirit of the invention. Although the invention has been disclosed in connection with specific preferred aspects or embodiments, it should be understood that the invention as claimed should not be unduly limited to such specific aspects or embodiments.

Although the foregoing subject matter has been described in some detail by way of illustration and example for purposes of clarity of understanding, it will be understood by those skilled in the art that certain changes and modifications can be practiced within the scope of the appended claims.

### DEFINITIONS

“Mild traumatic brain injury (mTBI)” or “concussion”—According to the Mild TBI Committee of the American Congress of Rehabilitation Medicine, revised by the World Health Organization (WHO), mTBI can be defined by a Glasgow Coma Scale score between 13 and 15 at 30 minutes post-injury, and one or more of the following symptoms: <30 min loss of consciousness; <24 hours post-traumatic amnesia (PTA); impaired mental state at time of accident (confusion, disorientation, etc.); and/or transient neurological deficit. mTBI, or concussion, may be a brain injury caused by a blow to the head or a violent shaking of the head and body. This occurs from a mild blow to the head, either with or without loss of consciousness and can lead to temporary cognitive symptoms. Symptoms may include headache, confusion, lack of coordination, memory loss, nausea, vomiting, dizziness, ringing in the ears, sleepiness, and excessive fatigue. In some cases, mTBI can be characterized as an absence of contusions or bruises in a brain image (e.g., MRI or CT scan images) associated with the mild traumatic brain injury.

“Clinical Domain(s)” can refer to the categories of various types of healthcare services provided to patients. Examples of clinical domains with respect to mild traumatic brain injury (mTBI) or concussion may include, but are not limited to, headache, sleep-wake, cervicogenic (i.e., neck), ocular (i.e., vision), vestibular (i.e., balance), behavioral, cognitive, and physiologic.

“Genotype” can refer to the genetic constitution of an individual organism. The genotype can refer to the genetic material passed between generations.

“Phenotype” can refer to the observable characteristics or traits of an organism. For example, the phenotype can refer to the set of observable characteristics of an individual resulting from the interaction of its genotype with the environment.

“Patient recovery phenotypes (PRP)” with respect to mild traumatic brain injury (mTBI) or concussion can refer to a grouping of mTBIs or concussions that demonstrate similar recoveries and/or some mutual similarities in other data attributes (such as demographics, injury characteristics, symptoms and treatment regimens. Examples of PRPs with respect to mTBI or concussion may include, but are not limited to, 1) a short recovery female sport related concussion and 2) a long recovery male adult motor vehicle crash.

“Predicted recovery metrics (PRMs)” with respect to mild traumatic brain injury (mTBI) or concussion can refer to the potential recovery timelines by clinical domain based on assessment of previous concussion cases and associated PRPs derived. Examples of PRMs with respect to mTBI or concussion may include, but are not limited to, vestibular recovery over time

“Symptom qualifiers” can refer to the presence, absence, state, and/or condition of any symptom with respect to mild traumatic brain injury (mTBI) or concussion. These are evaluated by the patient and/or their parent on a 0-6 rating scale (0=presence of symptom; 1-3=mild symptoms; 4-5=moderate symptom; 6=severe symptoms). Examples of symptom qualifiers with respect to mTBI or concussion may include, but are not limited to, headache=4; dizziness=2. There are 35 symptoms which our healthcare system will measure during a concussion recovery.

“A,” “an,” and “the” refer to “one or more” when used in this application, including the claims. Thus, for example, reference to “a subject” includes a plurality of subjects, unless the context clearly is to the contrary (e.g., a plurality of subjects), and so forth.

### List of Embodiments

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A computer-implemented method for predicting a plurality of treatment options for treatment of a traumatic brain injury for a subject, the computer-implemented method comprising: (a) receiving a plurality of attributes of the subject, wherein the plurality of attributes is related to the traumatic brain injury; and (b) applying a machine learning model to the plurality of attributes to predict (i) a clinical outcome comprising a traumatic brain injury, and (ii) the plurality of treatment options for treatment of the traumatic brain injury.

Embodiment 2. The computer-implemented method of Embodiment 1, wherein the receiving of the plurality of attributes is via a graphical user interface (GUI) of an electronic device.

Embodiment 3. The computer-implemented method of Embodiment 2, wherein the subject or a medical professional enters the plurality of attributes using the GUI.

Embodiment 4. The computer-implemented method of any one of Embodiments 1-3, wherein the receiving the plurality of attributes is performed autonomously.

Embodiment 5. The computer-implemented method of any one of Embodiments 1-4, wherein the receiving the plurality of attributes is via a wearable device.

Embodiment 6. The computer-implemented method of Embodiment 5, wherein the wearable device comprises a bracelet, a computer, a mobile device, an epidermal, an earbud, a headphone, a fabric, a shoe, an implant, glasses, a tracker, a belt, a sock, a shirt, or a ring.

Embodiment 7. The computer-implemented method of any one of Embodiments 1-6, wherein the plurality of attributes comprises past medical events of the subject.

Embodiment 8. The computer-implemented method of any one of Embodiments 1-7, wherein the plurality of attributes indicates a presence or absence of a symptom in the subject.

Embodiment 9. The computer-implemented method of any one of Embodiments 1-8, wherein the plurality of attributes indicates a severity or mildness of a symptom in the subject.

Embodiment 10. The computer-implemented method of any one of Embodiments 1-6, wherein the plurality of attributes comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, physical exam results, wearable physiologic data, imaging data, history of previous recovery trajectories and treatments, heart rate variability, physical activity data, blood pressure, perspiration, calories burned, steps walked, body temperature, or any combination thereof of the subject.

Embodiment 11. The computer-implemented method of any one of Embodiments 1-10, further comprising classifying the traumatic brain injury as a concussion.

Embodiment 12. The computer-implemented method of Embodiment 11, further comprising classifying the concussion as a concussion phenotype.

Embodiment 13. The computer-implemented method of Embodiment 12, wherein the concussion phenotype comprises a persistent concussion.

Embodiment 14. The computer-implemented method of any one of Embodiments 1-13, further comprising generating a probability that the traumatic brain injury is a concussion.

Embodiment 15. The computer-implemented method of any one of Embodiments 1-14, further comprising generating a plurality of probabilities for a plurality of concussion phenotypes of the traumatic brain injury.

Embodiment 16. The computer-implemented method of any one of Embodiments 1-15, further comprising predicting, by the machine learning model, a recovery timeline for the subject, based at least in part on the plurality on attributes.

Embodiment 17. The computer-implemented method of any one of Embodiments 1-16, further comprising selecting a treatment in the plurality of treatment options.

Embodiment 18. The computer-implemented method of Embodiment 17, wherein the treatment is personalized to the subject.

Embodiment 19. The computer-implemented method of Embodiment 17 or Embodiment 18, further comprising delivering or administering the treatment to the subject.

Embodiment 20. The computer-implemented method of any one of Embodiments 1-19, wherein the plurality of attributes comprises activity data of the subject, wherein the activity data relates to whether the subject has adhered to a current treatment.

Embodiment 21. The computer-implemented method of Embodiment 20, further comprising selecting a treatment in the plurality of treatment options for the subject based on at least in part on the activity data of the subject.

Embodiment 22. The computer-implemented method of Embodiment 21, wherein the treatment is different from a previous treatment delivered to the subject for the traumatic brain injury.

Embodiment 23. The computer-implemented method of Embodiment 21, wherein the treatment is delivered or administered to the subject with a duration or a frequency that is different than a previous duration or a previous frequency of a previous treatment delivered to the subject for the traumatic brain injury.

Embodiment 24. The computer-implemented method of Embodiment 21, wherein the treatment is performed by the subject with the duration or the frequency that is different than the previous duration or the previous frequency of a previous treatment delivered to the subject for the traumatic brain injury.

Embodiment 25. The computer-implemented method of any one of Embodiments 1-24, further comprising training the machine learning model by: (a) receiving a dataset comprising (i) a plurality of attributes of a plurality of reference subjects, and (ii) a plurality of clinical outcomes for the plurality of subjects; and (b) processing a reference dataset using the machine learning model to generate a plurality of outputs and (ii) updating a parameter of the machine learning model based on a loss function, wherein the loss function is based on the plurality of clinical outcomes and the plurality of outputs, wherein the plurality of outputs is indicative of a plurality of clinical outcome predictions.

Embodiment 26. The computer-implemented method of Embodiment 25, wherein the plurality of outputs parameterizes the plurality of clinical outcome predictions.

Embodiment 27. A computer-implemented method for training a machine learning model, comprising: (a) receiving a dataset comprising (i) a plurality of attributes of a plurality of reference subjects and (ii) a plurality of clinical outcomes for the plurality of reference subjects that received a plurality of traumatic brain injury treatments; and (b) training the machine learning model by (i) processing the dataset using the machine learning model to generate a plurality of outputs and (ii) updating a parameter of the machine learning model based on a loss function, wherein the loss function is based on the plurality of clinical outcomes and the plurality of outputs, wherein the plurality of outputs is indicative of an effectiveness of the plurality of traumatic brain injury treatments for the plurality of reference subjects.

Embodiment 28. The computer-implemented method of Embodiment 27, wherein the plurality of attributes comprises past medical events of the plurality of reference subjects.

Embodiment 29. The computer-implemented method of Embodiment 27 or Embodiment 28, wherein the plurality of attributes indicates a presence or absence of a symptom in the plurality of reference subjects.

Embodiment 30. The computer-implemented method of any one of Embodiments 27-29, wherein the plurality of attributes indicates a severity or mildness of a symptom in the plurality of reference subjects.

Embodiment 31. The computer-implemented method of any one of Embodiments 27-30, wherein the plurality of attributes comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, demographic characteristics, injury characteristics, physical exam results, wearable physiologic data, imaging data, history of previous recovery trajectories and treatments, or any combination thereof of the subject.

Embodiment 32. A computer-implemented method for training a machine learning model, comprising: (a) receiving a dataset comprising (i) a plurality of attributes of a plurality of reference subjects and (ii) a plurality of clinical outcomes for the plurality of reference subjects that were afflicted with a traumatic brain injury; and (b) training the machine learning model by (i) processing the dataset using the machine learning model to generate a plurality of outputs and (ii) updating a parameter of the machine learning model based on a loss function, wherein the loss function is based on the plurality of clinical outcomes and the plurality of outputs, wherein the plurality of outputs is indicative of a recovery phenotype of the traumatic brain injury for the plurality of reference subjects.

Embodiment 33. The computer-implemented method of Embodiment 32, wherein the plurality of outputs comprises a plurality of latent representations for the plurality of attributes for the plurality of reference subjects.

Embodiment 34. The computer-implemented method of Embodiment 33, further comprising clustering the plurality of latent representations identify the recovery phenotype for the plurality of reference subjects.

Embodiment 35. The computer-implemented method of any one of Embodiments 32-34, further comprising applying the machine learning model to a subject not among the plurality of reference subjects to classify the recovery phenotype of the subject.

Embodiment 36. The computer-implemented method of any one of Embodiments 32-35, wherein the plurality of attributes indicates a presence or absence of a symptom in the plurality of reference subjects.

Embodiment 37. The computer-implemented method of any one of Embodiments 32-36, wherein the plurality of attributes indicates a severity or mildness of a symptom in the plurality of reference subjects.

Embodiment 38. The computer-implemented method of any one of Embodiments 32-37, wherein the plurality of attributes comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, demographic characteristics, injury characteristics, physical exam results, wearable physiologic data, imaging data, history of previous recovery trajectories and treatments, or any combination thereof of the subject.

Embodiment 39. The computer-implemented method of any one of Embodiments 32-38, wherein the recovery phenotype comprises a concussion recovery phenotype.

Embodiment 40. The computer-implemented method of Embodiment 39, wherein the concussion recovery phenotype comprises a persistent concussion.

Embodiment 41. The computer-implemented method of any one of Embodiments 32-40, further comprising generating a probability that the recovery phenotype is a concussion recovery phenotype.

Embodiment 42. A computer-implemented method for optimizing a treatment for a traumatic brain injury in a subject, the method comprising: (a) receiving activity data of the subject, wherein the subject has received a treatment for the traumatic brain injury, and wherein the activity data relates to whether the subject has adhered to the treatment; and (b) applying a machine learning model to the activity data to predict whether the subject should continue the treatment or switch to a different treatment.

Embodiment 43. The computer-implemented method of Embodiment 42, wherein the receiving of the activity data is via a graphical user interface (GUI) of an electronic device.

Embodiment 44. The computer-implemented method of Embodiment 43, wherein the subject or a medical professional enters the plurality of attributes using the GUI.

Embodiment 45. The computer-implemented method of any one of Embodiments 42-44, wherein the receiving the plurality of attributes is performed autonomously.

Embodiment 46. The computer-implemented method of any one of Embodiments 42-45, wherein the receiving the plurality of attributes is via a wearable device.

Embodiment 47. The computer-implemented method of Embodiment 46, wherein the wearable device comprises a bracelet, a computer, a mobile device, an epidermal, an earbud, a headphone, a fabric, a shoe, an implant, glasses, a tracker, a belt, a sock, a shirt, or a ring.

Embodiment 48. The computer-implemented method of any one of Embodiments 42-47, wherein the activity data relates to whether the subject has adhered to a current treatment.

Embodiment 49. The computer-implemented method of any one of Embodiments 42-48, wherein the activity data indicates a presence or absence of a symptom in the subject.

Embodiment 50. The computer-implemented method of any one of Embodiments 42-49, wherein the activity data indicates a severity or mildness of a symptom in the subject.

Embodiment 51. The computer-implemented method of any one of Embodiments 42-50, wherein the activity data comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, history of previous recovery trajectories and treatments, or any combination thereof of the subject.

Embodiment 52. The computer-implemented method of any one of Embodiments 42-51, further comprising delivering or administering the different treatment to the subject.

Embodiment 53. The computer-implemented method of any one of Embodiments 42-52, wherein the different treatment is different from the treatment in a duration or a frequency.

Embodiment 54. The computer-implemented method of any one of Embodiments 42-53, further comprising training the machine learning model by: (a) receiving a dataset comprising (i) a plurality of attributes of a plurality of subjects, (ii) a plurality of activity data that relates to whether the subject has adhered to the treatment, and (iii) a plurality of clinical outcomes for the plurality of subjects; and (b) training the machine learning model by (i) processing the dataset using the machine learning model to generate a plurality of outputs and (ii) updating a parameter of the machine learning model based on a loss function, wherein the loss function is based on the plurality of clinical outcomes and the plurality of outputs, wherein the plurality of outputs comprises or parameterizes a plurality of clinical outcome predictions.

Embodiment 55. A computer-implemented method for generating an expected clinical outcome of a subject having a concussion, comprising: (a) receiving a plurality of attributes of the subject, wherein the plurality of attributes is related to a traumatic brain injury; and (b) applying a machine learning model to the plurality of attributes to predict a recovery timeline and an uncertainty value associated with the recovery timeline.

Embodiment 56. The computer-implemented method of Embodiment 55, wherein the recovery timeline comprises a timeline of one or more symptoms.

Embodiment 57. The computer-implemented method of Embodiment 55 or Embodiment 56, wherein the recovery timeline comprises a timeline for one or more concussion phenotypes.

Embodiment 58. The computer-implemented method of any one of Embodiments 55-57, wherein the recovery timeline comprises a plurality of uncertainty values.

Embodiment 59. The computer-implemented method of any one of Embodiments 51-54, wherein the receiving of the plurality of attributes is via a graphical user interface (GUI) of an electronic device.

Embodiment 60. The computer-implemented method of Embodiment 59, wherein the subject or a medical professional enters the plurality of attributes using the GUI.

Embodiment 61. The computer-implemented method of any one of Embodiments 55-60, wherein the receiving the plurality of attributes is performed autonomously.

Embodiment 62. The computer-implemented method of any one of Embodiments 55-61, wherein the receiving the plurality of attributes is via a wearable device.

Embodiment 63. The computer-implemented method of Embodiment 62, wherein the wearable device comprises a bracelet, a computer, a mobile device, an epidermal, an earbud, a headphone, a fabric, a shoe, an implant, glasses, a tracker, a belt, a sock, a shirt, or a ring.

Embodiment 64. The computer-implemented method of any one of Embodiments 55-63, wherein the plurality of attributes comprises past medical events of the subject.

Embodiment 65. The computer-implemented method of any one of Embodiments 55-64, wherein the plurality of attributes indicates a presence or absence of a symptom in the subject.

Embodiment 66. The computer-implemented method of any one of Embodiments 55-65, wherein the plurality of attributes indicates a severity or mildness of a symptom in the subject.

Embodiment 67. The computer-implemented method of any one of Embodiments 55-66, wherein the plurality of attributes comprises patient demographics, patient medical and family history, clinical symptom presentation from behavioral characteristics, cervicogenic characteristics, cognitive characteristics, headache characteristics, physiologic characteristics, ocular characteristics, sleep wake characteristics and vestibular characteristics, history of previous recovery trajectories and treatments, or any combination thereof of the subject or any combination thereof of the subject.

Embodiment 68. The computer-implemented method of any one of Embodiments 55-67, further comprising training the machine learning model by: (a) receiving a dataset comprising a plurality of attributes and a plurality of recovery timelines for a plurality of reference subjects, wherein the plurality of attributes and the plurality of time-varying clinical outcomes are related to a traumatic brain injury; and (b) training the machine learning model by processing the plurality of attributes using the machine learning model to generate a plurality of outputs and updating a parameter of the machine learning model based on a loss function, wherein the loss function is based on the plurality of recovery timelines and the plurality of outputs, wherein the plurality of outputs indicates a recovery timeline and an uncertainty value associated with the recovery timeline.

Embodiment 69. A platform comprising: (a) a client device comprising at least one processor, an operating system configured to perform executable instructions, a memory, and a computer program including instructions executable by the client device to create an application comprising a software module for receiving a plurality of attributes of a subject, wherein the plurality of attributes is related to a traumatic brain injury of the subject; and (b) a server comprising at least one processor, an operating system configured to perform executable instructions, a memory, and a computer program including instructions executable by the client device to create an application comprising a software module for applying a machine learning model to the plurality of attributes to predict (i) a clinical outcome comprising a traumatic brain injury, and (ii) a plurality of treatment options for treatment of the traumatic brain injury.

Embodiment 70. The platform of Embodiment 69, wherein the client device comprises a mobile electronic device.

Embodiment 71. The platform of Embodiment 69 or Embodiment 70, wherein the plurality of attributes comprises one or more recovery statistics of the subject.

Embodiment 72. The platform of any one of Embodiments 69-71, wherein the one or more recovery statistics of the subject are configured to be received from the subject.

Embodiment 73. The platform of any one of Embodiments 69-72, wherein the application further comprises a video player configured to provide one or more instructional videos for performing one or more exercises for treating the traumatic brain injury of the subject.

Embodiment 74. A computer-implemented system comprising a computing device comprising at least one processor, an operating system configured to perform executable instructions, a memory, and a computer program including instructions executable by the computing device to create an application comprising: (a) a software module configured to receive a plurality of attributes of the subject, wherein the plurality of attributes is related to a traumatic brain injury; and (b) a software module configured to apply a machine learning model to the plurality of attributes to predict (i) a clinical outcome comprising a traumatic brain injury, (ii) a recovery phenotype of the traumatic brain injury, or (iii) a plurality of treatment options for treatment of the traumatic brain injury.

Embodiment 75. Non-transitory computer-readable storage media encoded with a computer program including instructions executable by one or more processors to generate a selection of treatment options for a subject comprising: (a) a database, in a computer memory, comprising a plurality of attributes of the subject, wherein the plurality of attributes is related to a traumatic brain injury; and (b) a software module configured to apply a machine learning model to the plurality of attributes to predict (i) a clinical outcome comprising a traumatic brain injury, (ii) a recovery phenotype of the traumatic brain injury, or (iii) a plurality of treatment options for treatment of the traumatic brain injury.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A computer-implemented method for predicting a plurality of treatment options for treatment of a traumatic brain injury for a subject, the computer-implemented method comprising:
(a) receiving a plurality of attributes of the subject, wherein the plurality of attributes is related to the traumatic brain injury; and
(b) applying a machine learning model to the plurality of attributes to predict (i) a clinical outcome comprising the traumatic brain injury, and (ii) the plurality of treatment options for treatment of the traumatic brain injury.

2. The computer-implemented method of claim 1, wherein the receiving of the plurality of attributes is via a graphical user interface (GUI) of an electronic device.

3. The computer-implemented method of claim 2, wherein the subject or a medical professional enters the plurality of attributes using the GUI.

4. The computer-implemented method of claim 1, wherein the receiving the plurality of attributes is via a wearable device.

5. The computer-implemented method of claim 1, wherein the plurality of attributes comprises past medical events of the subject.

6. The computer-implemented method of claim 1, wherein the plurality of attributes indicates a presence or absence of a symptom in the subject, a severity or mildness of a symptom in the subject, or any combination thereof.

7. The computer-implemented method of claim 1, further comprising classifying the traumatic brain injury as a concussion; and classifying the concussion as a concussion phenotype, wherein the concussion phenotype comprises a persistent concussion.

8. The computer-implemented method of claim 7, further comprising generating a probability that the traumatic brain injury is the concussion or a plurality of probabilities for a plurality of concussion phenotypes of the traumatic brain injury.

9. The computer-implemented method of claim 1, further comprising predicting, by the machine learning model, a recovery timeline for the subject, based at least in part on the plurality of attributes.

10. The computer-implemented method of claim 1, further comprising selecting a treatment in the plurality of treatment options, wherein the treatment is personalized to the subject and the treatment is delivered or administered to the subject.

11. The computer-implemented method of claim 1, wherein the plurality of attributes comprises activity data of the subject, wherein the activity data relates to whether the subject has adhered to a current treatment.

12. The computer-implemented method of claim 11, further comprising selecting a treatment in the plurality of treatment options for the subject based at least in part on the activity data of the subject.

13. The computer-implemented method of any one of claims 1-12, further comprising training the machine learning model by:
(a) receiving a dataset comprising (i) a plurality of attributes of a plurality of reference subjects, and (ii) a plurality of clinical outcomes for the plurality of subjects; and
(b) processing a reference dataset using the machine learning model to generate a plurality of outputs, wherein the plurality of outputs parameterizes the plurality of clinical outcome predictions, and (ii) updating a parameter of the machine learning model based on a loss function, wherein the loss function is based on the plurality of clinical outcomes and the plurality of outputs, wherein the plurality of outputs is indicative of a plurality of clinical outcome predictions.

14. A computer-implemented method for training a machine learning model, comprising:
(a) receiving a dataset comprising (i) a plurality of attributes of a plurality of reference subjects and (ii) a plurality of clinical outcomes for the plurality of reference subjects that received a plurality of traumatic brain injury treatments; and
(b) training the machine learning model by (i) processing the dataset using the machine learning model to generate a plurality of outputs and (ii) updating a parameter of the machine learning model based on a loss function, wherein the loss function is based on the plurality of clinical outcomes and the plurality of outputs, wherein the plurality of outputs is indicative of an effectiveness of the plurality of traumatic brain injury treatments for the plurality of reference subjects.

15. The computer-implemented method of claim 14, wherein the plurality of attributes comprises past medical events of the plurality of reference subjects.

16. The computer-implemented method of claim 14 or 15, wherein the plurality of attributes indicates a presence or absence of a symptom in the plurality of reference subjects, a severity or mildness of a symptom in the plurality of reference subjects, or any combination thereof.

17. A computer-implemented method for training a machine learning model, comprising:
(a) receiving a dataset comprising (i) a plurality of attributes of a plurality of reference subjects and (ii) a plurality of clinical outcomes for the plurality of reference subjects that were afflicted with a traumatic brain injury; and
(b) training the machine learning model by (i) processing the dataset using the machine learning model to generate a plurality of outputs and (ii) updating a parameter of the machine learning model based on a loss function, wherein the loss function is based on the plurality of clinical outcomes and the plurality of outputs, wherein the plurality of outputs is indicative of a recovery phenotype of the traumatic brain injury for the plurality of reference subjects.

18. The computer-implemented method of claim 17, wherein the plurality of outputs comprises a plurality of latent representations for the plurality of attributes for the plurality of reference subjects; and further comprising clustering the plurality of latent representations identify the recovery phenotype for the plurality of reference subjects.

19. The computer-implemented method of claim 18, further comprising applying the machine learning model to a subject not among the plurality of reference subjects to classify the recovery phenotype of the subject wherein the plurality of attributes indicates a severity or mildness of a symptom in the plurality of reference subjects.

20. The computer-implemented method of claim 17, wherein the plurality of attributes indicates a presence or absence of a symptom in the plurality of reference subjects, a severity or mildness of a symptom in the plurality of reference subjects, or any combination thereof.

21. The computer-implemented method of claim 17, wherein the recovery phenotype comprises a concussion recovery phenotype, and wherein the concussion recovery phenotype comprises a persistent concussion.

22. The computer-implemented method of claim 21, further comprising generating a probability that the recovery phenotype is the concussion recovery phenotype.

23. A computer-implemented method for optimizing a treatment for a traumatic brain injury in a subject, the method comprising:
(a) receiving activity data of the subject, wherein the subject has received a treatment for the traumatic brain injury, and wherein the activity data relates to whether the subject has adhered to the treatment; and
(b) applying a machine learning model to the activity data to predict whether the subject should continue the treatment or switch to a different treatment.

24. The computer-implemented method of claim 23, wherein the receiving of the activity data is via a graphical user interface (GUI) of an electronic device.

25. The computer-implemented method of claim 24, wherein the subject or a medical professional enters the plurality of attributes using the GUI.

26. The computer-implemented method of claim 23, wherein the receiving the plurality of attributes is via a wearable device.

27. The computer-implemented method of claim 23, wherein the activity data relates to whether the subject has adhered to a current treatment.

28. The computer-implemented method of claim 23, wherein the activity data indicates a presence or absence of a symptom in the subject, a severity or mildness of a symptom in the subject, or any combination thereof.

29. The computer-implemented method of any one of claims 23-28, further comprising training the machine learning model by:
(a) receiving a dataset comprising (i) a plurality of attributes of a plurality of subjects, (ii) a plurality of activity data that relates to whether the subject has adhered to the treatment, and (iii) a plurality of clinical outcomes for the plurality of subjects; and
(b) training the machine learning model by (i) processing the dataset using the machine learning model to generate a plurality of outputs and (ii) updating a parameter of the machine learning model based on a loss function, wherein the loss function is based on the plurality of clinical outcomes and the plurality of outputs, wherein the plurality of outputs comprises or parameterizes a plurality of clinical outcome predictions.

30. A computer-implemented method for generating an expected clinical outcome of a subject having a concussion, comprising:
(a) receiving a plurality of attributes of the subject, wherein the plurality of attributes is related to a traumatic brain injury; and
(b) applying a machine learning model to the plurality of attributes to predict a recovery timeline and an uncertainty value associated with the recovery timeline.

31. The computer-implemented method of claim 30, wherein the recovery timeline comprises a timeline of one or more symptoms, a timeline for one or more concussion phenotypes, a plurality of uncertainty values, or any combination thereof.

32. The computer-implemented method of claim 30, wherein the receiving of the plurality of attributes is via a graphical user interface (GUI) of an electronic device.

33. The computer-implemented method of claim 32, wherein the subject or a medical professional enters the plurality of attributes using the GUI.

34. The computer-implemented method of claim 30, wherein the receiving the plurality of attributes is via a wearable device.

35. The computer-implemented method of claim 30, wherein the plurality of attributes comprises past medical events of the subject.

36. The computer-implemented method of claim 30, wherein the plurality of attributes indicates a presence or absence of a symptom in the subject, a severity or mildness of a symptom in the subject, or any combination thereof.

37. The computer-implemented method of any one of claims 30-36, further comprising training the machine learning model by:
(a) receiving a dataset comprising a plurality of attributes and a plurality of recovery timelines for a plurality of reference subjects, wherein the plurality of attributes and the plurality of time-varying clinical outcomes are related to a traumatic brain injury; and
(b) training the machine learning model by processing the plurality of attributes using the machine learning model to generate a plurality of outputs and updating a parameter of the machine learning model based on a loss function, wherein the loss function is based on the plurality of recovery timelines and the plurality of outputs, wherein the plurality of outputs indicates a recovery timeline and an uncertainty value associated with the recovery timeline.

38. A platform comprising:
(a) a client device comprising at least one processor, an operating system configured to perform executable instructions, a memory, and a computer program including instructions executable by the client device to create an application comprising a software module for receiving a plurality of attributes of a subject, wherein the plurality of attributes is related to a traumatic brain injury of the subject; and
(b) a server comprising at least one processor, an operating system configured to perform executable instructions, a memory, and a computer program including instructions executable by the client device to create an application comprising a software module for applying a machine learning model to the plurality of attributes to predict (i) a clinical outcome comprising a traumatic brain injury, and (ii) a plurality of treatment options for treatment of the traumatic brain injury.

39. The platform of claim 38, wherein the client device comprises a mobile electronic device.

40. The platform of claim 38, wherein the plurality of attributes comprises one or more recovery statistics of the subject; and wherein the one or more recovery statistics of the subject are configured to be received from the subject.

41. The platform of claim 38, wherein the application further comprises a video player configured to provide one or more instructional videos for performing one or more exercises for treating the traumatic brain injury of the subject.

42. A computer-implemented system comprising a computing device comprising at least one processor, an operating system configured to perform executable instructions, a memory, and a computer program including instructions executable by the computing device to create an application comprising:
a) a software module configured to receive a plurality of attributes of the subject, wherein the plurality of attributes is related to a traumatic brain injury; and
b) a software module configured to apply a machine learning model to the plurality of attributes to predict (i) a clinical outcome comprising a traumatic brain injury, (ii) a recovery phenotype of the traumatic brain injury, or (iii) a plurality of treatment options for treatment of the traumatic brain injury.

43. Non-transitory computer-readable storage media encoded with a computer program including instructions executable by one or more processors to generate a selection of treatment options for a subject comprising:
a) a database, in a computer memory, comprising a plurality of attributes of the subject, wherein the plurality of attributes is related to a traumatic brain injury; and
b) a software module configured to apply a machine learning model to the plurality of attributes to predict (i) a clinical outcome comprising a traumatic brain injury, (ii) a recovery phenotype of the traumatic brain injury, or (iii) a plurality of treatment options for treatment of the traumatic brain injury.
